(19) United States
(12) Patent Application Publication
(10) Pub. No.: US 2018/0026203 A1
(43) Pub. Date: Jan. 25, 2018

(54) COMPOUND, MATERIAL FOR ORGANIC ELECTROLUMINESCENT ELEMENT, INK COMPOSITION, ORGANIC ELECTROLUMINESCENT ELEMENT, AND ELECTRONIC DEVICE

(71) Applicant: IDEMITSU KOSAN CO., LTD., Chiyoda-ku (JP)

(72) Inventors: Kiyoshi IKEDA, Sodegaura-shi (JP); Hironori KAWAKAMI, Katsushika-ku (JP)

(73) Assignee: IDEMITSU KOSAN CO., LTD., Chiyoda-ku (JP)

(21) Appl. No.: 15/550,678

(22) PCT Filed: Feb. 12, 2016
(86) PCT No.: PCT/JP2016/054193

§ 371 Date: Aug. 11, 2017

(30) Foreign Application Priority Data
Feb. 13, 2015 (JP) 2015-026854

(51) Int. Cl. H01L 51/00; C09K 11/02; C09K 11/06; H01L 51/56; C09D 11/52; C07D 491/048; C07D 405/14; H01L 51/50; C07D 403/14; C09D 11/50

(52) U.S. Cl. CPC H01L 51/0072; C07D 403/14; C09K 11/025; C09K 11/06; C09D 11/50; C09D 11/52; C07D 491/048; H01L 51/0058; C07D 405/14; H01L 51/0073; H01L 51/5016; H01L 51/56; C09K 2211/185; C09K 2211/1007; C09K 2211/1029

(57) ABSTRACT

Organic EL devices with improved performance and electronic devices including the organic EL devices are provided, and compounds represented by formula (1), materials for organic electroluminescence devices, ink compositions which realize the organic EL devices are provided:

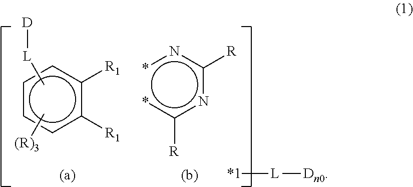

### TECHNICAL FIELD

The present invention relates to compounds, materials for organic electroluminescence devices, ink compositions, organic electroluminescence devices, and electronic devices.

### BACKGROUND ART

Organic electroluminescence devices have been known, in which an organic thin film layer including a light emitting layer is disposed between an anode and a cathode, and the energy of exciton generated by the recombination of hole and electron which are injected into a light emitting layer is converted into light.

Utilizing its advantages as the spontaneous emitting device, the organic electroluminescence device has been expected to provide a light emitting device excellent in the emission efficiency, the image quality, the power consumption, and the freedom of design.

Many researches have been made on the applications of organic electroluminescence device to display, etc. because of its possibility of a wide selection of emission colors by using various emitting materials in a light emitting layer. Particularly, the research on the materials which emit three primary red, green, and blue colors has been made most actively, and the intensive research has been made to improve their properties.

### CITATION LIST

### Patent Literature

Patent Literature: WO 2012/086170

### SUMMARY OF INVENTION

### Technical Problem

An object of the present invention is to provide a high performance organic EL device and an electronic device comprising it. Another object is to provide a compound, a material for organic electroluminescence device and an ink composition which realize a high performance organic EL device and an electronic device comprising it.

### Solution to Problem

In an aspect of the invention, the following (1) to (5) are provided: (1) a compound represented by formula (1):

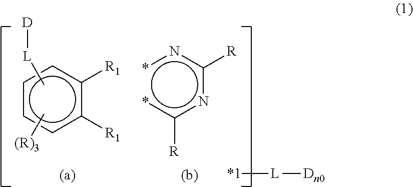

wherein:

two R₁'s are single bonds which are respectively bonded to two asterisks * of formula (b), or two R₁'s are bonded to each other to form a 5-membered ring, a 6-membered ring, or a fused ring, wherein two adjacent ring carbon atoms of the 5-membered ring, the 6-membered ring, or the fused ring are respectively bonded to two asterisks * of formula (b);

an unshared ring atom of the 5-membered ring is selected from a carbon atom, an oxygen atom, and a sulfur atom, wherein the carbon atom has two R's;

when the 6-membered ring is a non-aromatic ring, an unshared ring atom of the non-aromatic ring is selected from a carbon atom, an oxygen atom, and a sulfur atom, wherein the carbon atom has two R's;

when the 6-membered ring is an aromatic ring, an unshared ring atom of the aromatic ring is selected from a carbon atom and a nitrogen atom, and the carbon atom has one R;

when the fused ring comprises a non-aromatic ring, an unshared ring atom of the non-aromatic ring is selected from a carbon atom, an oxygen atom, and a sulfur atom, wherein the carbon atom has two R's;

when the fused ring comprises an aromatic ring, an unshared ring atom of the aromatic ring is selected from a carbon atom and a nitrogen atom, and the carbon atom has one R;

each of R's is independently a hydrogen atom, a substituent, or a single bond bonded to *¹-L-Dₙ₀, and neighboring or adjacent two R's may be bonded to each other to form a ring which may have one or more substituents R, provided that at least one of R's in formula (1) is a single bond bonded to *¹-L-Dₙ₀, this showing that when R, for example R in A-R, is a single bond bonded to *¹-L-Dₙ₀, the resulting compound is A-L-Dₙ₀;

each of L's is independently a single bond or a substituted or unsubstituted arylene group having 6 to 60 ring carbon atoms, when L is a single bond n0 is 1, and when L is a substituted or unsubstituted arylene group having 6 to 60 ring carbon atoms n0 is an integer of 1 to 10;

D is a monovalent residue of a structure represented by formula (2) which comprises at least one carbazole structure:

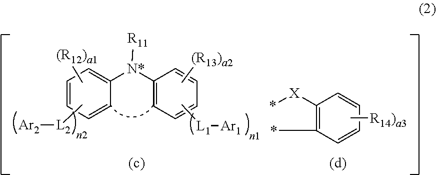

wherein:

a dotted line means that two carbon atoms at both ends thereof are bonded to each other via a single bond or the two carbon atoms are not bonded;

each of L₁ and L₂ is independently a single bond or a substituted or unsubstituted arylene group having 6 to 60 ring carbon atoms;

each of Ar₁ and Ar₂ is independently a substituted or unsubstituted monovalent residue of an aromatic hydrocarbon ring having 6 to 60 ring carbon atoms;

each of n1 and n2 is independently an integer of 0 to 4;

formula (d) bonds to adjacent two ring carbon atoms of at least one ring selected from two benzene rings bonded to N* and the aromatic hydrocarbon rings represented by Ar₁ and Ar₂, provided that when formula (d) is bonded to the aromatic hydrocarbon ring represented by Ar₁ or Ar₂, each of Ar₁ and Ar₂ represents a monovalent residue of a structure formed by the aromatic hydrocarbon ring and formula (d) which are bonded to each other;

X is O, S, PR₁₅, SiR₁₆R₁₇, CR₁₈R₁₉, or NR₂₀, and when two or more formulae (d) are bonded, two or more X's may be the same or different;

each of R₁₁ to R₂₀ is independently a hydrogen atom or a substituent; and

each of a1, a2, and a3 is independently an integer of 0 to 4;

(2) a material for organic electroluminescence device comprising the compound of (1);
(3) an ink composition comprising a solvent and the compound of (1) dissolved in the solvent;
(4) an organic electroluminescence device which comprises an organic thin film layer comprising one or more layers between a cathode and an anode, wherein the organic thin film layer comprises a light emitting layer and at least one layer of the organic thin film layer comprises the compound of (1); and
(5) an electronic device comprising the organic electroluminescence device of (4).

### Advantageous Effects of Invention

The present invention provides an organic EL device having improved properties and an electronic device comprising it. The present invention further provides a compound, a material for organic electroluminescence device, and an ink composition which realize such an organic EL device and an electronic device.

### BRIEF DESCRIPTION OF DRAWINGS

FIG. 1 is a schematic view of the structure of the organic EL device according to one embodiment of the invention.

### DESCRIPTION OF EMBODIMENTS

The term of “XX to YY carbon atoms” referred to by “a substituted or unsubstituted group ZZ having XX to YY carbon atoms” used herein is the number of carbon atoms of the unsubstituted group ZZ and does not include any carbon atom in the substituent of the substituted group ZZ.

The term of “XX to YY atoms” referred to by “a substituted or unsubstituted group ZZ having XX to YY atoms” used herein is the number of atoms of the unsubstituted group ZZ and does not include any atom in the substituent of the substituted group ZZ.

The number of “ring carbon atoms” referred to herein means the number of the carbon atoms included in the atoms which are members forming the ring itself of a compound in which a series of atoms is bonded to form the ring (for example, a monocyclic compound, a fused ring compound, a cross-linked compound, a carbocyclic compound, and a heterocyclic compound). If the ring has a substituent, the carbon atom in the substituent is not included in the ring carbon atom. The same applies to the number of “ring carbon atom” described below, unless otherwise noted. For example, a benzene ring has 6 ring carbon atoms, a naphthalene ring has 10 ring carbon atoms, a pyridinyl group has 5 ring carbon atoms, and a furanyl group has 4 ring carbon atoms. If a benzene ring or a naphthalene ring has, for example, an alkyl substituent, the carbon atom in the alkyl substituent is not counted as the ring carbon atom of the benzene or naphthalene ring. In case of a fluorene ring to which a fluorene substituent is bonded (inclusive of a spirofluorene ring), the carbon atom in the fluorene substituent is not counted as the ring carbon atom of the fluorene ring.

The number of “ring atom” referred to herein means the number of the atoms which are members forming the ring itself (for example, a monocyclic ring, a fused ring, and a ring assembly) of a compound in which a series of atoms is bonded to form the ring (for example, a monocyclic compound, a fused ring compound, a cross-linked compound, a carbocyclic compound, and a heterocyclic compound). The atom not forming the ring and the atom in a substituent, if the ring is substituted, are not counted as the ring atom. The same applies to the number of “ring atoms” described below, unless otherwise noted. For example, a pyridine ring has 6 ring atoms, a quinazoline ring has 10 ring atoms, and a furan ring has 5 ring atoms. The hydrogen atom on the ring carbon atom of a pyridine ring or a quinazoline ring and the atom in a substituent are not counted as the ring atom. In case of a fluorene ring to which a fluorene substituent is bonded (inclusive of a spirofluorene ring), the atom in the fluorene substituent is not counted as the ring atom of the fluorene ring.

The definition of “hydrogen atom” used herein includes isotopes different in the neutron numbers, i.e., light hydrogen (protium), heavy hydrogen (deuterium), and tritium.

The terms of “heteroaryl group” and “heteroarylene group” used herein means a group having at least one hetero atom as a ring atom. The heteroatom is preferably at least one selected from a nitrogen atom, an oxygen atom, a sulfur atom, a silicon atom, and a selenium atom, more preferably at least one selected from a nitrogen atom, an oxygen atom, and a sulfur atom.

The substituent referred to by “a substituent” or “a substituted or unsubstituted” used herein is selected from Group (A) consisting of an alkyl group having 1 to 50, preferably 1 to 18, more preferably 1 to 8 carbon atoms; a cycloalkyl group having 3 to 50, preferably 3 to 10, more preferably 3 to 8, still more preferably 5 or 6 ring carbon atoms; an aryl group having 6 to 50, preferably 6 to 25, more preferably 6 to 18 ring carbon atoms; an aralkyl group having 7 to 51, preferably 7 to 30, more preferably 7 to 20 carbon atoms which includes an aryl group having 6 to 50, preferably 6 to 25, more preferably 6 to 18 ring carbon atoms; an amino group; a mono- or di-substituted amino group, wherein the substituent is selected from an alkyl group having 1 to 50, preferably 1 to 18, more preferably 1 to 8 carbon atoms and an aryl group having 6 to 50, preferably 6 to 25, more preferably 6 to 18 ring carbon atoms; an alkoxy group having an alkyl group having 1 to 50, preferably 1 to 18, more preferably 1 to 8 carbon atoms; an aryloxy group having an aryl group having 6 to 50, preferably 6 to 25, more preferably 6 to 18 ring carbon atoms; a mono-, di- or tri-substituted silyl group, wherein the substituent is selected from an alkyl group having 1 to 50, preferably 1 to 18, more preferably 1 to 8 carbon atoms and an aryl group having 6 to 50, preferably 6 to 25, more preferably 6 to 18 ring carbon atoms; a heteroaryl group having 5 to 50, preferably 5 to 24, more preferably 5 to 13 ring atoms; a haloalkyl group having 1 to 50, preferably 1 to 18, more preferably 1 to 8 carbon atoms; a halogen atom selected from a fluorine atom, a chlorine atom, a bromine atom and an iodine atom; a cyano group; a nitro group; a substituted sulfonyl group, wherein the substituent is selected from an alkyl group having 1 to 50, preferably 1 to 18, more preferably 1 to 8 carbon atoms and an aryl group having 6 to 50, preferably 6 to 25, more preferably 6 to 18 ring carbon atoms; a di-substituted phosphoryl group, wherein the substituent is selected from an alkyl group having 1 to 50, preferably 1 to 18, more preferably 1 to 8 carbon atoms and an aryl group having 6 to 50, preferably 6 to 25, more preferably 6 to 18 ring carbon atoms; an alkylsulfonyloxy group; an arylsulfonyloxy group; an alkylcarbonyloxy group; an arylcarbonyloxy group; a boron-containing group; a zinc-containing group; a tin-containing group; a silicon-containing group; a magnesium-containing group; a lithium-containing group; a hydroxyl group; an alkyl-substituted or aryl-substituted carbonyl group; a carboxyl group; a vinyl group; a (meth)acryloyl group; an epoxy group; and an oxetanyl group.

These substituents may have the substituent mentioned above. The substituents may be bonded to each other to form a ring.

The term “unsubstituted” referred to by “a substituted or unsubstituted” means that a hydrogen atom is not substituted by the substituent mentioned above.

Of the above substituents, more preferred are selected from Group (B) consisting of an alkyl group having 1 to 50, preferably 1 to 18, more preferably 1 to 8 carbon atoms; a cycloalkyl group having 3 to 50, preferably 3 to 10, more preferably 3 to 8, still more preferably 5 or 6 ring carbon atoms; an aryl group having 6 to 50, preferably 6 to 25, more preferably 6 to 18 ring carbon atoms; a mono- or di-substituted amino group, wherein the substituent is selected from an alkyl group having 1 to 50, preferably 1 to 18, more preferably 1 to 8 carbon atoms and an aryl group having 6 to 50, preferably 6 to 25, more preferably 6 to 18 ring carbon atoms; a heteroaryl group having 5 to 50, preferably 5 to 24, more preferably 5 to 13 ring atoms; a halogen atom; and a cyano group.

In the present invention, the features which are defined as being preferred can be selected arbitrarily.

### Compound

The compound in an aspect of the invention will be described below.

The compound in an aspect of the invention is a compound represented by formula (1) (hereinafter also referred to as “compound (1)”):

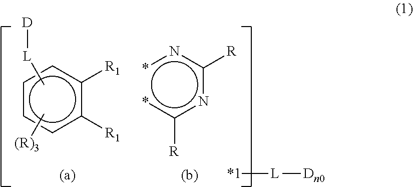

wherein:

two R₁'s are single bonds which are respectively bonded to two asterisks * of formula (b), or two R₁'s are bonded to each other to form a 5-membered ring, a 6-membered ring, or a fused ring, wherein two adjacent ring carbon atoms of the 5-membered ring, the 6-membered ring, or the fused ring are respectively bonded to two asterisks * of formula (b);

an unshared ring atom of the 5-membered ring is selected from a carbon atom, an oxygen atom, and a sulfur atom, wherein the carbon atom has two R's;

when the 6-membered ring is a non-aromatic ring, an unshared ring atom of the non-aromatic ring is selected from a carbon atom, an oxygen atom, and a sulfur atom, wherein the carbon atom has two R's;

when the 6-membered ring is an aromatic ring, an unshared ring atom of the aromatic ring is selected from a carbon atom and a nitrogen atom, and the carbon atom has one R;

when the fused ring comprises a non-aromatic ring, an unshared ring atom of the non-aromatic ring is selected from a carbon atom, an oxygen atom, and a sulfur atom, wherein the carbon atom has two R's; and

when the fused ring comprises an aromatic ring, an unshared ring atom of the aromatic ring is selected from a carbon atom and a nitrogen atom, and the carbon atom has one R;

The unshared ring atom referred to herein is a ring atom not shared with another ring. For example, Y and Z in formulae (6) to (16) are unshared ring atoms.

The aromatic ring referred to herein is a ring consisting of only a ring having aromaticity, for example, a benzene ring and a pyrimidine ring. The non-aromatic ring referred herein is a ring having no multiple bond, for example, a cyclopentane ring. For example, the central cyclopentane ring of fluorene is also referred to as a non-aromatic ring.

Each of R's is independently a hydrogen atom, a substituent, or a single bond bonded to *¹-L-Dₙ₀, and neighboring or adjacent two R's may be bonded to each other to form a ring which may have one or more substituents R, provided that at least one of R's in formula (1) is a single bond bonded to *¹-L-Dₙ₀.

Preferably, each R is independently selected from the following groups: a substituted or unsubstituted alkyl group having 1 to 50, preferably 1 to 18, more preferably 1 to 8 carbon atoms; a substituted or unsubstituted cycloalkyl group having 3 to 50, preferably 3 to 10, more preferably 3 to 8 ring carbon atoms; a substituted or unsubstituted aryl group (or aromatic hydrocarbon group, the same applies below) having 6 to 60, preferably 6 to 25, more preferably 6 to 18 ring carbon atoms; a substituted or unsubstituted aralkyl group having 7 to 61, preferably 7 to 25, more preferably 7 to 18 carbon atoms; amino group; a mono- or di-substituted amino group, wherein the substituent is selected from a substituted or unsubstituted alkyl group having 1 to 50, preferably 1 to 18, more preferably 1 to 8 carbon atoms and a substituted or unsubstituted aryl group having 6 to 60, preferably 6 to 25, more preferably 6 to 18 ring carbon atoms; a substituted or unsubstituted alkoxy group having an alkyl group having 1 to 50, preferably 1 to 18, more preferably 1 to 8 carbon atoms; a substituted or unsubstituted cycloalkoxy group having 3 to 50, preferably 3 to 10, more preferably 3 to 8 ring carbon atoms; a substituted or unsubstituted aryloxy group having an aryl group having 6 to 60, preferably 6 to 25, more preferably 6 to 18 ring carbon atoms; a substituted or unsubstituted alkylthio group having 1 to 50, preferably 1 to 18, more preferably 1 to 8 carbon atoms; a substituted or unsubstituted arylthio group having 6 to 60, preferably 6 to 25, more preferably 6 to 18 ring carbon atoms; a mono-, di- or tri-substituted silyl group, wherein the substituent is selected from a substituted or unsubstituted alkyl group having 1 to 50, preferably 1 to 18, more preferably 1 to 8 carbon atoms and a substituted or unsubstituted aryl group having 6 to 60, preferably 6 to 25, more preferably 6 to 18 ring carbon atoms; a substituted or unsubstituted heteroaryl group (or heterocyclic group, the same applies below) having 5 to 60, preferably 5 to 30, more preferably 5 to 26 ring atoms; a substituted or unsubstituted haloalkyl group having 1 to 50, preferably 1 to 18, more preferably 1 to 8 carbon atoms; a halogen atom, a cyano group, a nitro group, a sulfonyl group having a substituent selected from a substituted or unsubstituted alkyl group having 1 to 50, preferably 1 to 18, more preferably 1 to 8 carbon atoms and a substituted or unsubstituted aryl group having 6 to 60, preferably 6 to 25, more preferably 6 to 18 ring carbon atoms; a di-substituted phosphoryl group, wherein the substituent is selected from a substituted or unsubstituted alkyl group having 1 to 50, preferably 1 to 18, more preferably 1 to 8 carbon atoms and a substituted or unsubstituted aryl group having 6 to 60, preferably 6 to 25, more preferably 6 to 18 ring carbon atoms; an alkylsulfonyloxy group; an arylsulfonyloxy group; an alkylcarbonyloxy group; an arylcarbonyloxy group; a boron-containing group; a zinc-containing group; a tin-containing group; a silicon-containing group; a magnesium-containing group; a lithium-containing group; a hydroxyl group; an alkyl-substituted or aryl-substituted carbonyl group; a carboxyl group; a vinyl group; a (meth)acryloyl group; an epoxy group; and an oxetanyl group.

Examples of the alkyl group include a methyl group, an ethyl group, a n-propyl group, an isopropyl group, a n-butyl group, an isobutyl group, a s-butyl group, a t-butyl group, a pentyl group (inclusive of isomeric groups), a hexyl group (inclusive of isomeric groups), a heptyl group (inclusive of isomeric groups), an octyl group (inclusive of isomeric groups), a nonyl group (inclusive of isomeric groups), a decyl group (inclusive of isomeric groups), an undecyl group (inclusive of isomeric groups), a dodecyl group (inclusive of isomeric groups), a tridecyl group, a tetradecyl group, an octadecyl group, a tetracosanyl group, and a tetracontanyl group. These groups may have a substituent.

More preferred are a methyl group, an ethyl group, a n-propyl group, an isopropyl group, a n-butyl group, an isobutyl group, a s-butyl group, a t-butyl group, a pentyl group (inclusive of isomeric groups), a hexyl group (inclusive of isomeric groups), a heptyl group (inclusive of isomeric groups), an octyl group (inclusive of isomeric groups), a nonyl group (inclusive of isomeric groups), a decyl group (inclusive of isomeric groups), an undecyl group (inclusive of isomeric groups), a dodecyl group (inclusive of isomeric groups), a tridecyl group, a tetradecyl group, and an octadecyl group. These groups may have a substituent.

Still more preferred are a methyl group, an ethyl group, a n-propyl group, an isopropyl group, a n-butyl group, an isobutyl group, a s-butyl group, a t-butyl group, a pentyl group (inclusive of isomeric groups), a hexyl group (inclusive of isomeric groups), a heptyl group (inclusive of isomeric groups), and an octyl group (inclusive of isomeric groups). These groups may have a substituent.

Examples of the cycloalkyl group include a cyclopropyl group, a cyclobutyl group, a cyclopentyl group, a cyclohexyl group, a cycloheptyl group, a cyclooctyl group, and an adamantyl group. These groups may have a substituent.

More preferred are a cyclopentyl group and a cyclohexyl group. These groups may have a substituent.

Examples of the aryl group include a phenyl group, a naphthyl group, a naphthylphenyl group, a biphenylyl group, a terphenylyl group, a quarterphenylyl group, a quinquephenylyl group, an acenaphthylenyl group, an anthryl group, a benzanthryl group, an aceanthryl group, a phenanthryl group, a benzophenanthryl group, a phenalenyl group, a fluorenyl group, a 9,9′-spirobifluorenyl group, a benzofluorenyl group, a dibenzofluorenyl group, a picenyl group, a pentaphenyl group, a pentacenyl group, a pyrenyl group, a chrysenyl group, a benzochrysenyl group, a s-indacenyl group, an as-indacenyl group, a fluoranthenyl group, a benzofluoranthenyl group, a tetracenyl group, a triphenylenyl group, a benzotriphenylenyl group, a perylenyl group, a coronyl group, a dibenzanthryl group, a 9,9-dimethylfluorenyl group, and a 9,9-diphenylfluorenyl group. These groups may have a substituent.

More preferred are a phenyl group, a naphthyl group, a biphenylyl group, a terphenylyl group, a phenanthryl group, a benzophenanthryl group, a fluorenyl group, a 9,9′-spirobifluorenyl group, a benzofluorenyl group, a dibenzofluorenyl group, a chrysenyl group, a benzochrysenyl group, a s-indacenyl group, an as-indacenyl group, a triphenylenyl group, a benzotriphenylenyl group, an anthryl group, a 9,9-dimethylfluorenyl group, and a 9,9-diphenylfluorenyl group. These groups may have a substituent.

Still more preferred are a phenyl group, a biphenylyl group, a terphenylyl group, a naphthyl group, a phenanthryl group, a fluorenyl group, a 9,9′-spirobifluorenyl group, a chrysenyl group, a triphenylenyl group, a 9,9-dimethylfluorenyl group, and a 9,9-diphenylfluorenyl group. These groups may have a substituent.

The heteroaryl group includes at least one, preferably 1 to 5, more preferably 1 to 3, and still more preferably 1 or 2 hetero atoms, for example, a nitrogen atom, a sulfur atom, an oxygen atom, and a phosphorus atom. Examples of the heteroaryl group include a pyrrolyl group, a furyl group, a thienyl group, a pyridyl group, a pyridazinyl group, a pyrimidinyl group, a pyrazinyl group, a triazinyl group, an imidazolyl group, an oxazolyl group, a thiazolyl group, a pyrazolyl group, an isoxazolyl group, an isothiazolyl group, an oxadiazolyl group, a thiadiazolyl group, a triazolyl group, a tetrazolyl group, an indolyl group, an isoindolyl group, a benzofuranyl group, an isobenzofuranyl group, a benzothiophenyl group, an isobenzothiophenyl group, an indolizinyl group, a quinolizinyl group, a quinolyl group, an isoquinolyl group, a cinnolyl group, a phthalazinyl group, a quinazolinyl group, a quinoxalinyl group, a benzimidazolyl group, a benzoxazolyl group, a benzothiazolyl group, an indazolyl group, a benzisoxazolyl group, a benzisothiazolyl group, a dibenzofuranyl group, a dibenzothiophenyl group, a carbazolyl group, a bicarbazolyl group, a phenanthridinyl group, an acridinyl group, a phenanthrolinyl group, a phenazinyl group, a phenothiazinyl group, a phenoxazinyl group, an azatriphenylenyl group, a diazatriphenylenyl group, a xanthenyl group, an azacarbazolyl group, an azadibenzofuranyl group, an azadibenzothiophenyl group, a benzofuranobenzothiophenyl group, a benzothienobenzothiophenyl group, a dibenzofuranonaphthyl group, a dibenzothienonaphthyl group, a dinaphthothienothiophenyl group, and a dinaphto[2′,3′:2,3:2′,3′:6,7]carbazolyl group. These groups may have a substituent.

More preferred are a pyridyl group, a pyridazinyl group, a pyrimidinyl group, a pyrazinyl group, a triazinyl group, an imidazolyl group, an indolyl group, an isoindolyl group, a benzofuranyl group, an isobenzofuranyl group, a benzothiophenyl group, an isobenzothiophenyl group, an indolizinyl group, a quinolizinyl group, a quinolyl group, an isoquinolyl group, a quinazolinyl group, a quinoxalinyl group, a benzimidazolyl group, a benzoxazolyl group, a benzothiazolyl group, a benzisoxazolyl group, a benzisothiazolyl group, a dibenzofuranyl group, a dibenzothiophenyl group, a carbazolyl group, a bicarbazolyl group, a phenanthridinyl group, an acridinyl group, a phenanthrolinyl group, an azatriphenylenyl group, a diazatriphenylenyl group, a xanthenyl group, an azacarbazolyl group, an azadibenzofuranyl group, and an azadibenzothiophenyl group. These groups may have a substituent.

Still more preferred are a pyridyl group, a pyrimidinyl group, a triazinyl group, a benzofuranyl group, an isobenzofuranyl group, a quinolyl group, an isoquinolyl group, a quinazolinyl group, a benzothiophenyl group, an isobenzothiophenyl group, an indolizinyl group, a dibenzofuranyl group, a dibenzothiophenyl group, a carbazolyl group, a bicarbazolyl group, an azatriphenylenyl group, a diazatriphenylenyl group, a xanthenyl group, an azacarbazolyl group, an azadibenzofuranyl group, and an azadibenzothiophenyl group. These groups may have a substituent.

Examples of the aralkyl group include those having the aryl group having 6 to 60 ring carbon atoms mentioned above. These groups may have a substituent. Preferred are aralkyl groups each having the aryl group having 6 to 25 ring carbon atoms mentioned above, and more preferred are aralkyl groups each having the aryl group having 6 to 18 ring carbon atoms mentioned above. These groups may have a substituent.

Examples of the mono- or di-substituted amino group include those having a substituent selected from the alkyl group having 1 to 50 carbon atoms and the aryl group having 6 to 60 ring carbon atoms, each described above, with a di-substituted amino group being preferred and a di-substituted amino group having a substituent selected from the aryl group mentioned above being more preferred. These groups may have a substituent. More preferred are mono- or di-substituted amino groups each having a substituent selected from the alkyl group having 1 to 18 carbon atoms and the aryl group having 6 to 25 ring carbon atoms, each described above. These groups may have a substituent. Still more preferred are mono- or di-substituted amino groups each having a substituent selected from the alkyl group having 1 to 8 carbon atoms and the aryl group having 6 to 18 ring carbon atoms, each described above. These groups may have a substituent.

Examples of the alkoxy group include those having the alkyl group having 1 to 50 carbon atoms mentioned above. These groups may have a substituent. More preferred are alkoxy groups each having the alkyl group having 1 to 18 carbon atoms mentioned above. These groups may have a substituent. Still more preferred are alkoxy groups each having the alkyl group having 1 to 8 carbon atoms mentioned above. These groups may have a substituent.

Examples of the cycloalkoxy group include those having the cycloalkyl group having 3 to 50 carbon atoms mentioned above. These groups may have a substituent.

Examples of the aryloxy group include those having the aryl group having 6 to 60 ring carbon atoms mentioned above. These groups may have a substituent. More preferred are aryloxy groups each having the aryl group having 6 to 25 ring carbon atoms mentioned above. These groups may have a substituent. Still more preferred are aryloxy groups each having the aryl group having 6 to 18 ring carbon atoms mentioned above, with a phenoxy group being preferred. These groups may have a substituent.

Examples of the alkylthio group include those having the alkyl group having 1 to 50 carbon atoms mentioned above. These groups may have a substituent. More preferred are alkylthio groups each having the alkyl group having 1 to 18 carbon atoms mentioned above. These groups may have a substituent. Still more preferred are alkylthio groups each having the alkyl group having 1 to 8 carbon atoms mentioned above. These groups may have a substituent.

Examples of the arylthio group include those having the aryl group having 6 to 60 ring carbon atoms mentioned above. These groups may have a substituent. More preferred are arylthio groups each having the aryl group having 6 to 25 ring carbon atoms mentioned above. These groups may have a substituent. Still more preferred are arylthio groups each having the aryl group having 6 to 18 ring carbon atoms mentioned above. These groups may have a substituent.

Examples of the mono-, di-, or trisubstituted silyl group include those having a substituent selected from the alkyl group having 1 to 50 carbon atoms and the aryl group having 6 to 60 ring carbon atoms, each described above. These groups may have a substituent. More preferred are mono-, di-, or trisubstituted silyl groups each having a substituent selected from the alkyl group having 1 to 18 carbon atoms and the aryl group having 6 to 25 ring carbon atoms, each described above. These groups may have a substituent. Still more preferred are mono-, di-, or trisubstituted silyl groups each having a substituent selected from the alkyl group having 1 to 8 carbon atoms and the aryl group having 6 to 18 ring carbon atoms, each described above. These groups may have a substituent. Specific examples thereof include a trimethylsilyl group, a triethylsilyl group, a t-butyldimethylsilyl group, a vinyldimethylsilyl group, a propyldimethylsilyl group, an isopropyldimethylsilyl group, a triphenylsilyl group, a phenyldimethylsilyl group, a t-butyldiphenylsilyl group, and a tritolylsilyl group. These groups may have a substituent.

Examples of the haloalkyl group include those derived from the alkyl group having 1 to 50 carbon atoms mentioned above by replacing one or more hydrogen atoms with a halogen atom, such as a fluorine atom, a chlorine atom, a bromine atom and an iodine atom. These groups may have a substituent. More preferred are those derived from the alkyl group having 1 to 18 carbon atoms mentioned above by replacing one or more hydrogen atoms with the halogen atom. These groups may have a substituent. Still more preferred are those derived from the alkyl group having 1 to 8 carbon atoms mentioned above by replacing one or more hydrogen atoms with the halogen atom. These groups may have a substituent. Specific examples include a trifluoromethyl group, a pentafluoroethyl group, and a heptafluoropropyl group.

Examples of the sulfonyl group include those having a substituent selected from the alkyl group having 1 to 50 carbon atoms and the aryl group having 6 to 60 ring carbon atoms, each described above. These groups may have a substituent. More preferred are sulfonyl groups each having a substituent selected from the alkyl group having 1 to 18 carbon atoms and the aryl group having 6 to 25 ring carbon atoms, each described above. These groups may have a substituent. Still more preferred are sulfonyl groups each having a substituent selected from the alkyl group having 1 to 8 carbon atoms and the aryl group having 6 to 18 ring carbon atoms, each described above. These groups may have a substituent.

Examples of the di-substituted phosphoryl group include those having a substituent selected from the alkyl group having 1 to 50 carbon atoms and the aryl group having 6 to 60 ring carbon atoms, each described above. These groups may have a substituent. More preferred are di-substituted phosphoryl groups each having a substituent selected from the alkyl group having 1 to 18 carbon atoms and the aryl group having 6 to 25 ring carbon atoms, each described above. These groups may have a substituent. Still more preferred are di-substituted phosphoryl groups each having a substituent selected from the alkyl group having 1 to 8 carbon atoms and the aryl group having 6 to 18 ring carbon atoms, each described above. These groups may have a substituent.

Examples of the alkylsulfonyloxy group, the arylsulfonyloxy group, the alkylcarbonyloxy group, the arylcarbonyloxy group, and the alkyl- or aryl-substituted carbonyl group include those having a substituent selected from the alkyl group and the aryl group mentioned above.

Preferably, each R is independently a hydrogen atom, a substituted or unsubstituted alkyl group having 1 to 50 carbon atoms, a substituted or unsubstituted cycloalkyl group having 3 to 50 ring carbon atoms, a substituted or unsubstituted alkoxy group having 1 to 50 carbon atoms, a substituted or unsubstituted aryl group having 6 to 60 ring carbon atoms, a substituted or unsubstituted heteroaryl group having 5 to 60 ring atoms, a substituted or unsubstituted amino group, a substituted or unsubstituted aryloxy group having 6 to 60 ring carbon atoms, a substituted silyl group, a substituted or unsubstituted haloalkyl group having 1 to 50 carbon atoms, a halogen atom, a cyano group, or a nitro group. Examples of these groups are as described above.

Each of L's is independently a single bond or a substituted or unsubstituted arylene group having 6 to 60 ring carbon atoms, when L is a single bond n0 is 1, and when L is a substituted or unsubstituted arylene group having 6 to 60 ring carbon atoms n0 is an integer of 1 to 10.

Examples of the arylene group having 6 to 60 ring carbon atoms for L include divalent redidues of the aryl groups mentioned above with respect to R.

n0 is preferably 1 or 2 and more preferably 1.

D is a monovalent residue of a structure represented by formula (2) which comprises at least one carbazole structure:

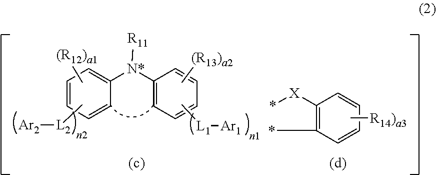

wherein:

a dotted line means that two carbon atoms at both ends thereof are bonded to each other via a single bond or the two carbon atoms are not bonded;

each of L₁ and L₂ is independently a single bond or a substituted or unsubstituted arylene group having 6 to 60 ring carbon atoms;

each of Ar₁ and Ar₂ is independently a substituted or unsubstituted monovalent residue of an aromatic hydrocarbon ring having 6 to 60 ring carbon atoms;

each of n1 and n2 is independently an integer of 0 to 4;

formula (d) bonds to adjacent two ring carbon atoms of at least one ring selected from two benzene rings bonded to N* and the aromatic hydrocarbon rings represented by Ar₁ and Ar₂, provided that when formula (d) is bonded to the aromatic hydrocarbon ring represented by Ar₁ or Ar₂, each of Ar₁ and Ar₂ represents a monovalent residue of a structure formed by the aromatic hydrocarbon ring and formula (d) which are bonded to each other;

X is O, S, PR₁₅, SiR₁₆R₁₇, CR₁₈R₁₉, or NR₂₀, and when two or more formulae (d) are bonded, two or more X's may be the same or different; and

each of R₁₁ to R₂₀ is independently a hydrogen atom or a substituent, wherein examples of the substituent represented by each of R₁₁ to R₂₀ are the same as those mentioned above with respect to R; and

each of a1, a2, and a3 is independently an integer of 0 to 4.

The compound represented by formula (1) is preferably represented by formula (3) or (4):

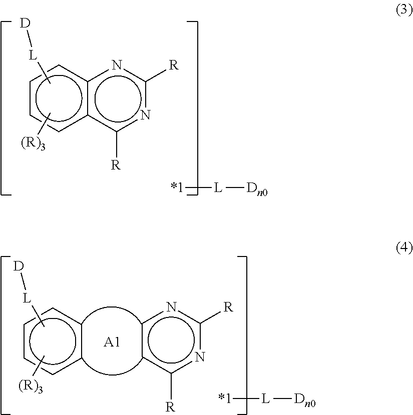

wherein:

R, L, D, and n0 are the same as defined in formula (1);

A₁ is a 5-membered ring, a 6-membered ring, or a fused ring;

an unshared ring atom of the 5-membered ring is selected from a carbon atom, an oxygen atom, and a sulfur atom, wherein the carbon atom has two R's;

when the 6-membered ring is a non-aromatic ring, an unshared ring atom of the non-aromatic ring is selected from a carbon atom, an oxygen atom, and a sulfur atom, wherein the carbon atom has two R's;

when the 6-membered ring is an aromatic ring, an unshared ring atom of the aromatic ring is selected from a carbon atom and a nitrogen atom, and the carbon atom has one R;

when the fused ring comprises a non-aromatic ring, an unshared ring atom of the non-aromatic ring is selected from a carbon atom, an oxygen atom, and a sulfur atom, wherein the carbon atom has two R's; and

when the fused ring comprises an aromatic ring, an unshared ring atom of the aromatic ring is selected from a carbon atom and a nitrogen atom, and the carbon atom has one R.

The structure which is formed by formula (a) and formula (b) in formula (1) is preferably represented by any of formulae (5) to (16):

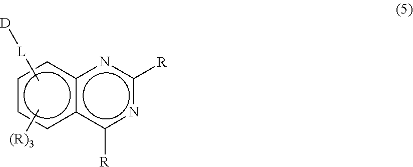

wherein R, L, and D are the same as defined in formula (1).

The structure represented by formula (5) is preferably represented by formula (5′):

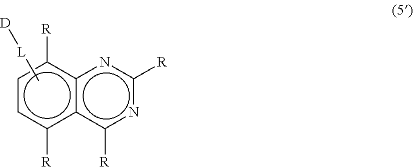

wherein R, L, and D are the same as defined in formula (1).

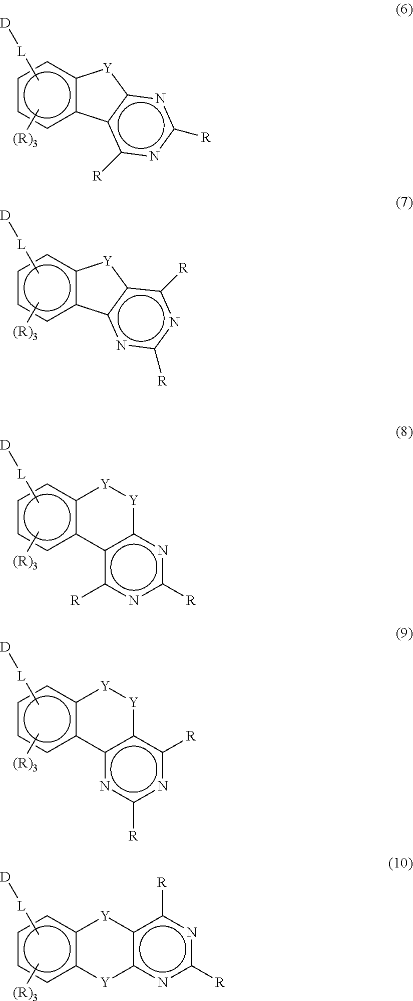

wherein:

Y is selected from a carbon atom, an oxygen atom, and a sulfur atom, wherein the carbon atom has two R's; and

R, L, and D are the same as defined in formula (1).

Of the above, the structure represented by formula (6) or (7) is preferred, which is more preferably represented by formula (6′) and (7′):

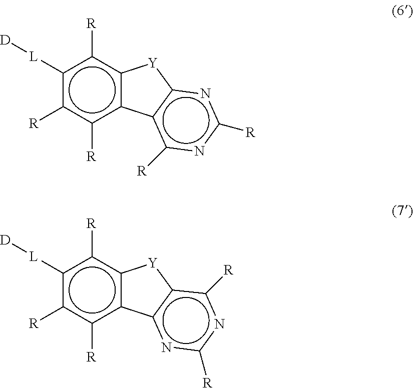

wherein:

Y is selected from a carbon atom, an oxygen atom, and a sulfur atom, wherein the carbon atom has two R's; and

R, L, and D are the same as defined in formula (1).

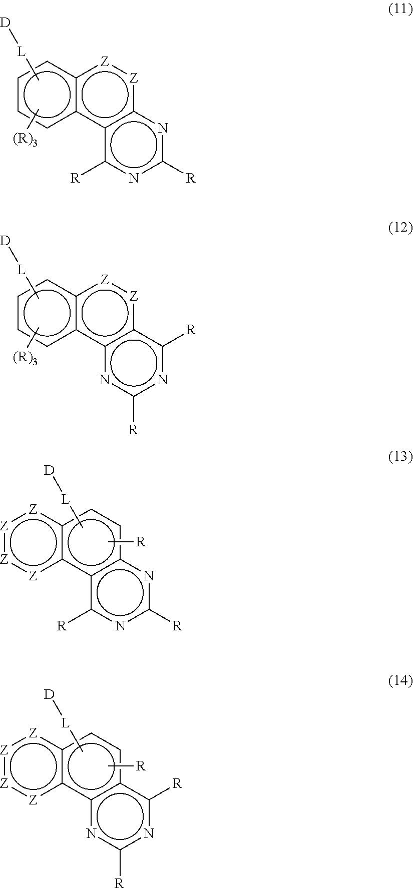

wherein:

Z is selected from a carbon atom and a nitrogen atom, wherein the carbon atom has one R; and

R, L, and D are the same as defined in formula (1).

Of the above, the compound represented by formula (11) or (12) is preferred, and the compound represented by formula (11) or (12), wherein one of R's on the pyrimidine ring is a single bond which is bonded to *¹-L-Dₙₒ is more preferred.

The structure represented by formula (11) or (12) is preferably represented by formula (11′) or (12′):

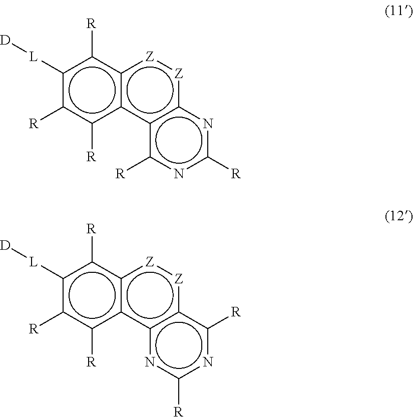

wherein:

Z is selected from a carbon atom and a nitrogen atom, wherein the carbon atom has one R; and

R, L, and D are the same as defined in formula (1).

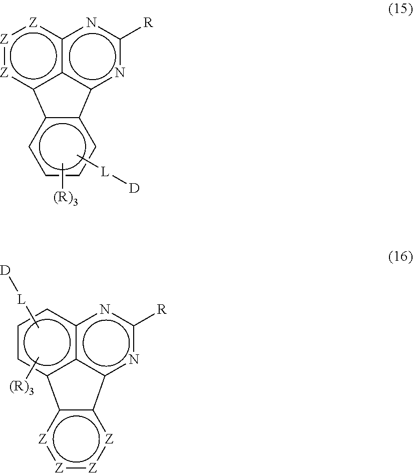

wherein:

Z is selected from a carbon atom and a nitrogen atom, wherein the carbon atom has one R; and

R, L, and D are the same as defined in formula (1).

In addition, the structure which is formed by formula (a) and formula (b) in formula (1) is preferably represented by any of the following formulae, wherein R, L, and D are the same as defined in formula (1):

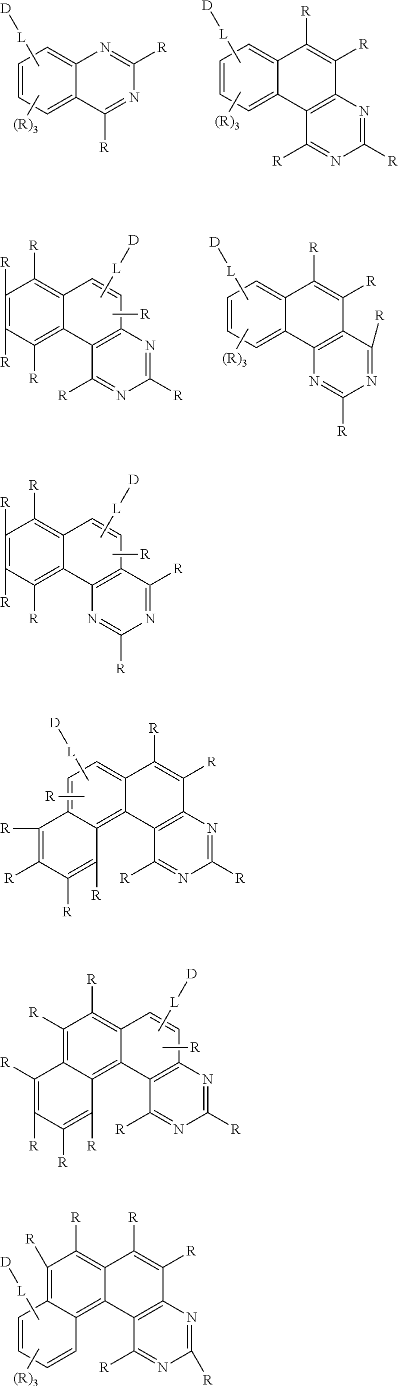

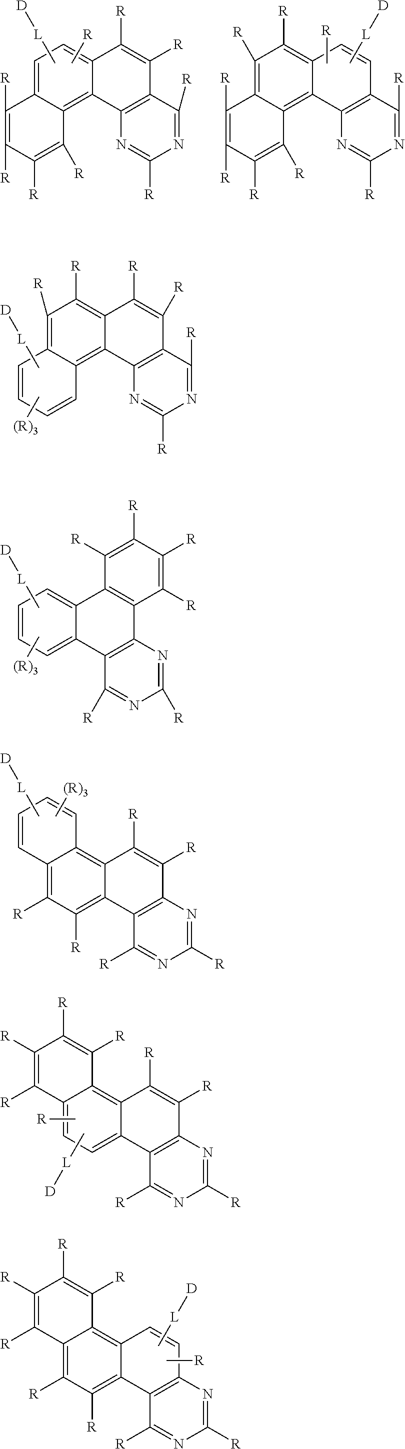

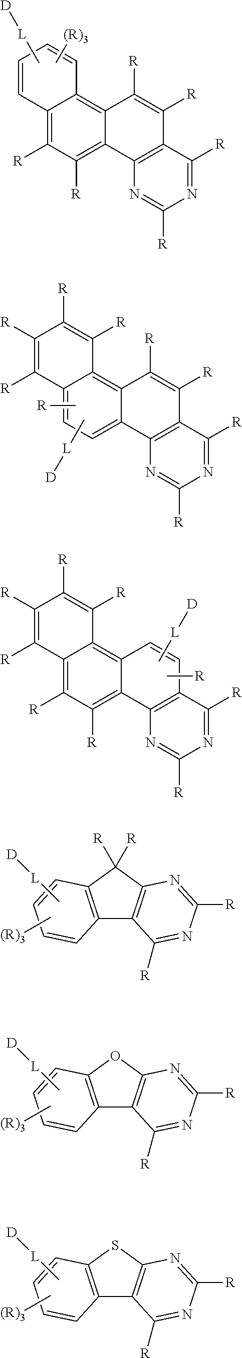

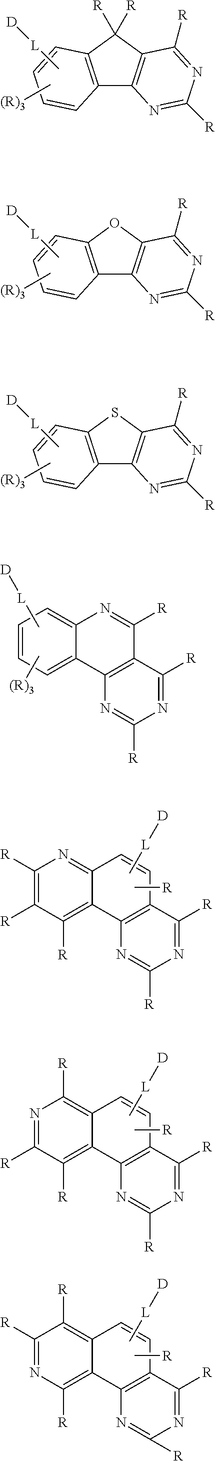

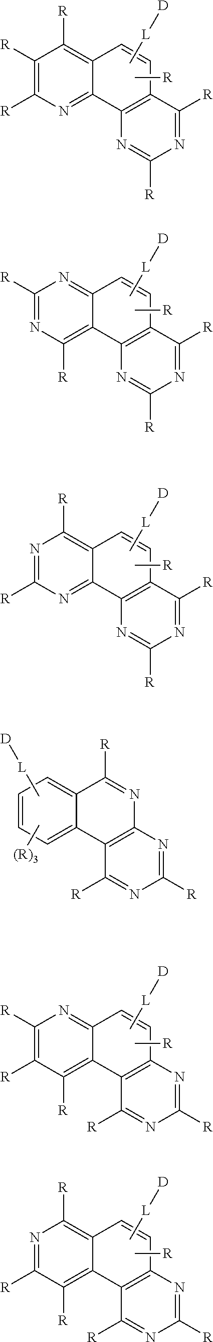

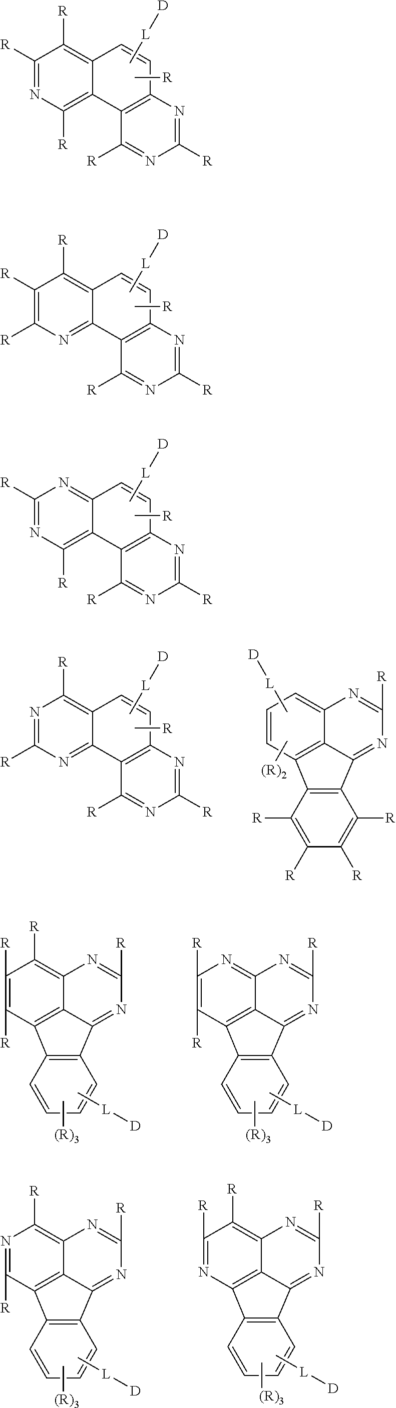

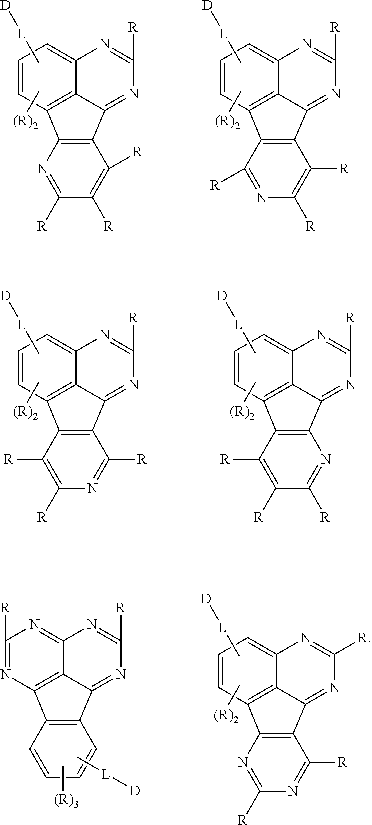

The structure of D represented by formula (2) is preferably represented by any of formulae (51) to (59):

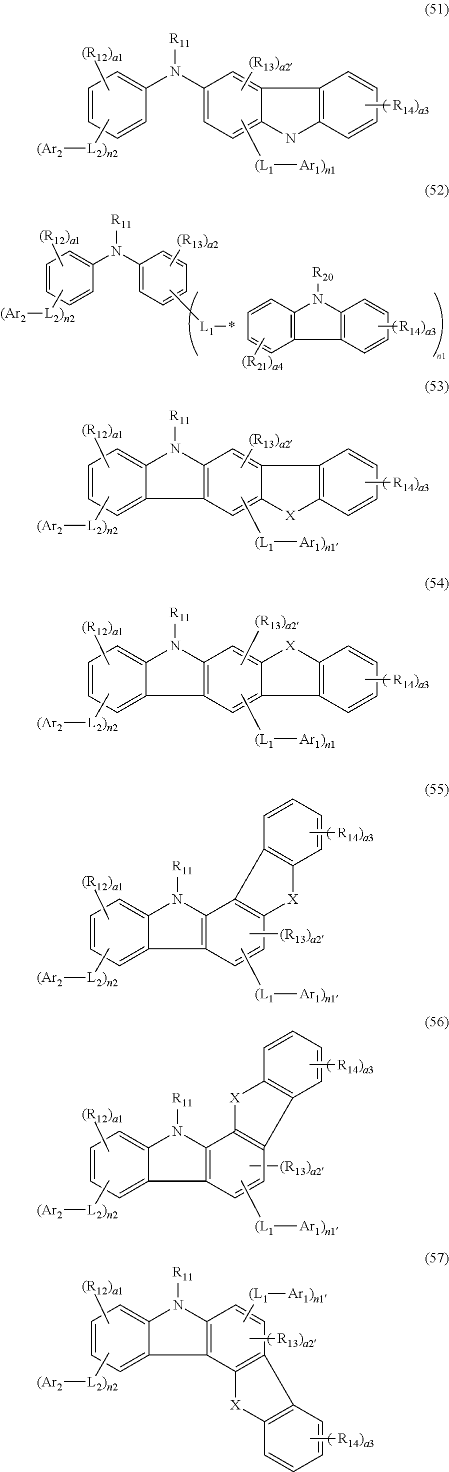

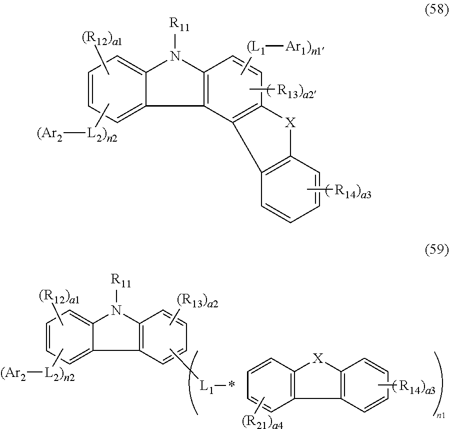

wherein:

X, R₁₁ to R₁₄, R₂₀, a1 to a3, n1 to n2, L₁, L₂, An, and Ar₂ are the same as defined above;

R₂₁ is a hydrogen atom or a substituent;

a4 is an integer of 0 to 4;

each of a2′ and n1′ is an integer of 0 to 2.

Examples of the substituent represented by R₂₁ are the same as those described above with respect to R.

In addition, the structure of D represented by formula (2) is preferably represented by formula (60) or (61):

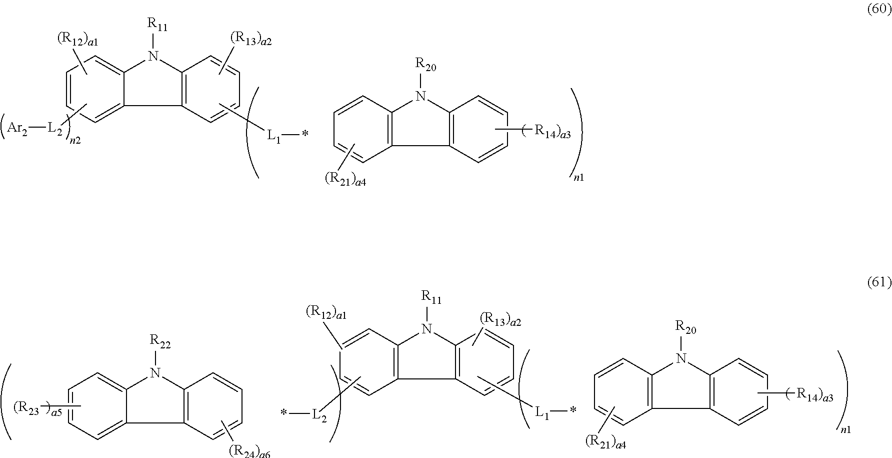

wherein:

R₁₁ to R₁₄, R₂₀, a1 to a3, n1, n2, L₁, L₂, and Ar₂ are the same as defined above;

each of R₂₁ to R₂₄ is a hydrogen atom or a substituent; and

each of a4 to a6 is an integer of 0 to 4.

Examples of the substituent represented by any of R₂₁ to R₂₄ are the same as those described above with respect to R.

Of the above, the structure represented by formula (60) is preferred and the structure represented by formula (60), wherein L₁ is a single bond, is more preferred.

Examples of the compound (1) are described below, although not limited thereto.

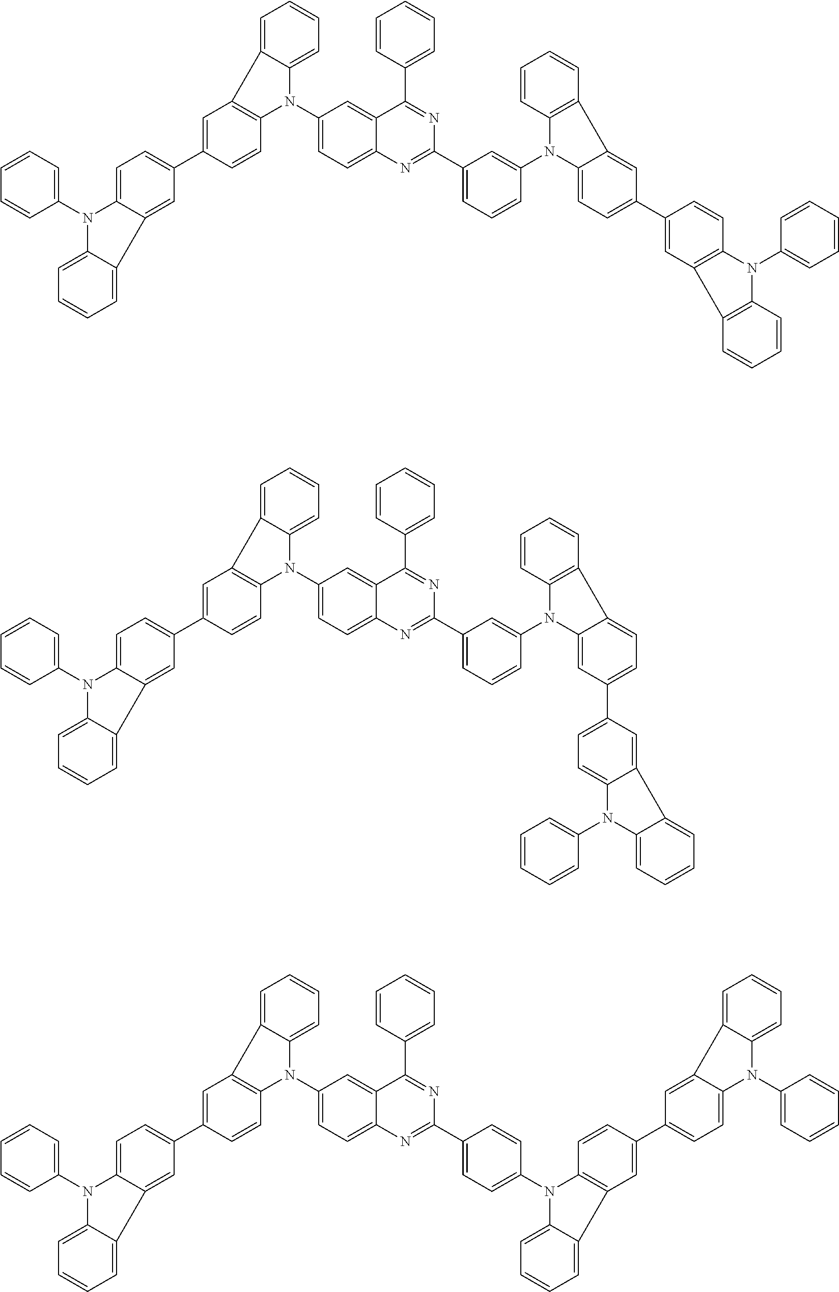 [SMILES: c1ccc(-c2nc(-c3cccc(-n4c5ccccc5c5cc(-c6ccc7c(c6)c6ccccc6n7-c6ccccc6)ccc54)c3)nc3ccc(-n4c5ccccc5c5cc(-c6ccc7c(c6)c6ccccc6n7-c6ccccc6)ccc54)cc23)cc1 ; c1ccc(-c2nc(-c3cccc(-n4c5ccccc5c5ccc(-c6ccc7c(c6)c6ccccc6n7-c6ccccc6)cc54)c3)nc3ccc(-n4c5ccccc5c5cc(-c6ccc7c(c6)c6ccccc6n7-c6ccccc6)ccc54)cc23)cc1 ; c1ccc(-c2nc(-c3ccc(-n4c5ccccc5c5cc(-c6ccc7c(c6)c6ccccc6n7-c6ccccc6)ccc54)cc3)nc3ccc(-n4c5ccccc5c5cc(-c6ccc7c(c6)c6ccccc6n7-c6ccccc6)ccc54)cc23)cc1]

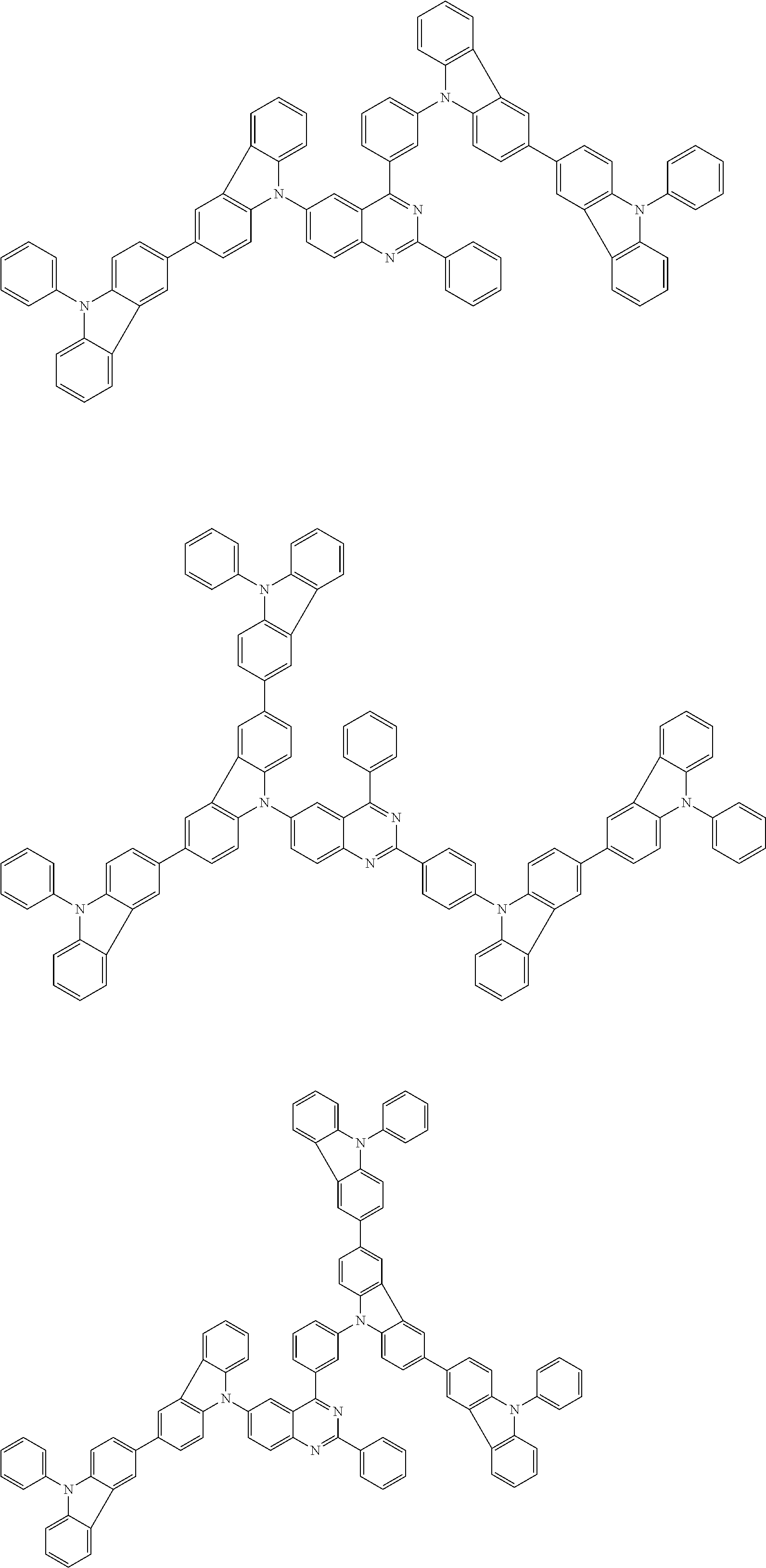

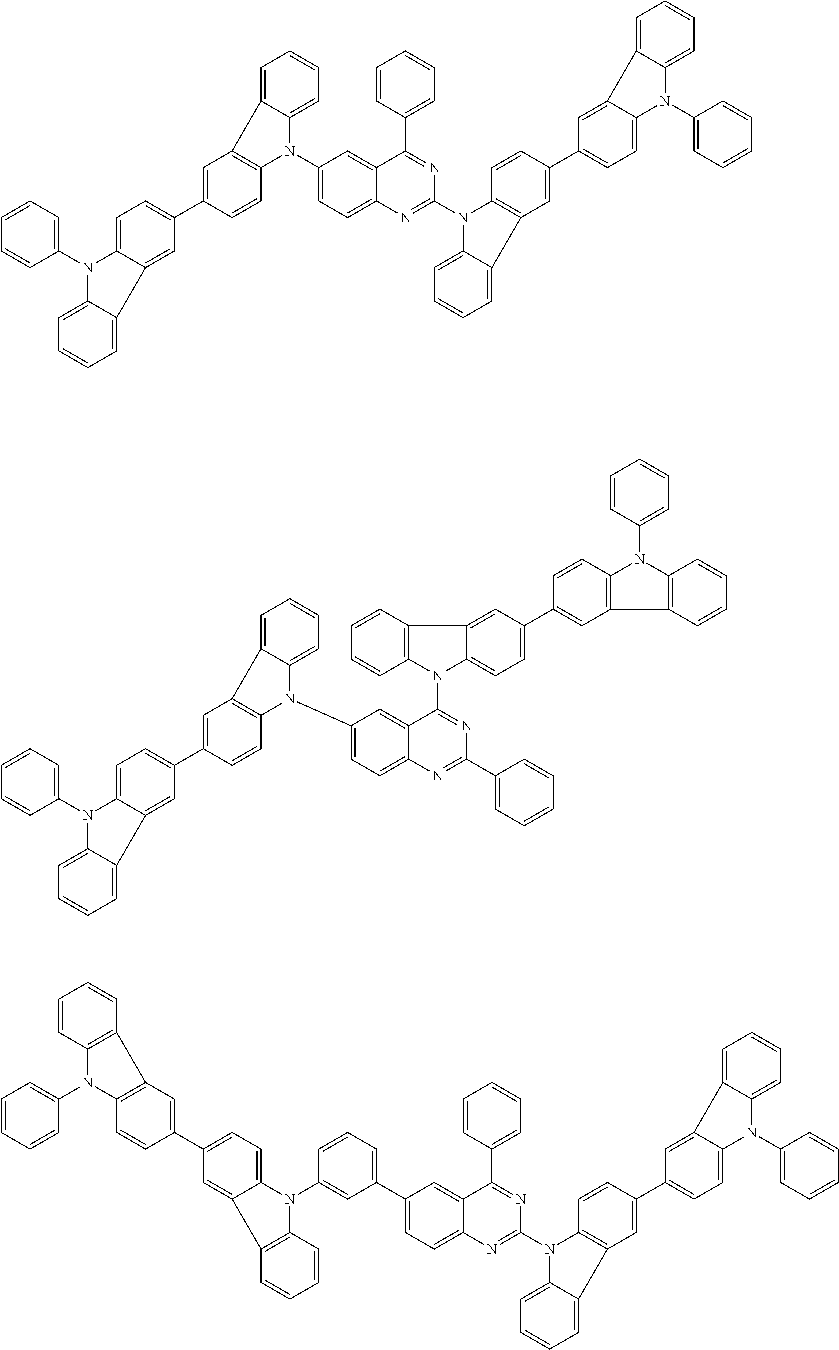 [SMILES: c1ccc(-c2nc(-n3c4ccccc4c4cc(-c5ccc6c(c5)c5ccccc5n6-c5ccccc5)ccc43)nc3ccc(-n4c5ccccc5c5cc(-c6ccc7c(c6)c6ccccc6n7-c6ccccc6)ccc54)cc23)cc1 ; c1ccc(-c2nc(-n3c4ccccc4c4cc(-c5ccc6c(c5)c5ccccc5n6-c5ccccc5)ccc43)c3cc(-n4c5ccccc5c5cc(-c6ccc7c(c6)c6ccccc6n7-c6ccccc6)ccc54)ccc3n2)cc1 ; c1ccc(-c2nc(-n3c4ccccc4c4cc(-c5ccc6c(c5)c5ccccc5n6-c5ccccc5)ccc43)nc3ccc(-c4cccc(-n5c6ccccc6c6cc(-c7ccc8c(c7)c7ccccc7n8-c7ccccc7)ccc65)c4)cc23)cc1]

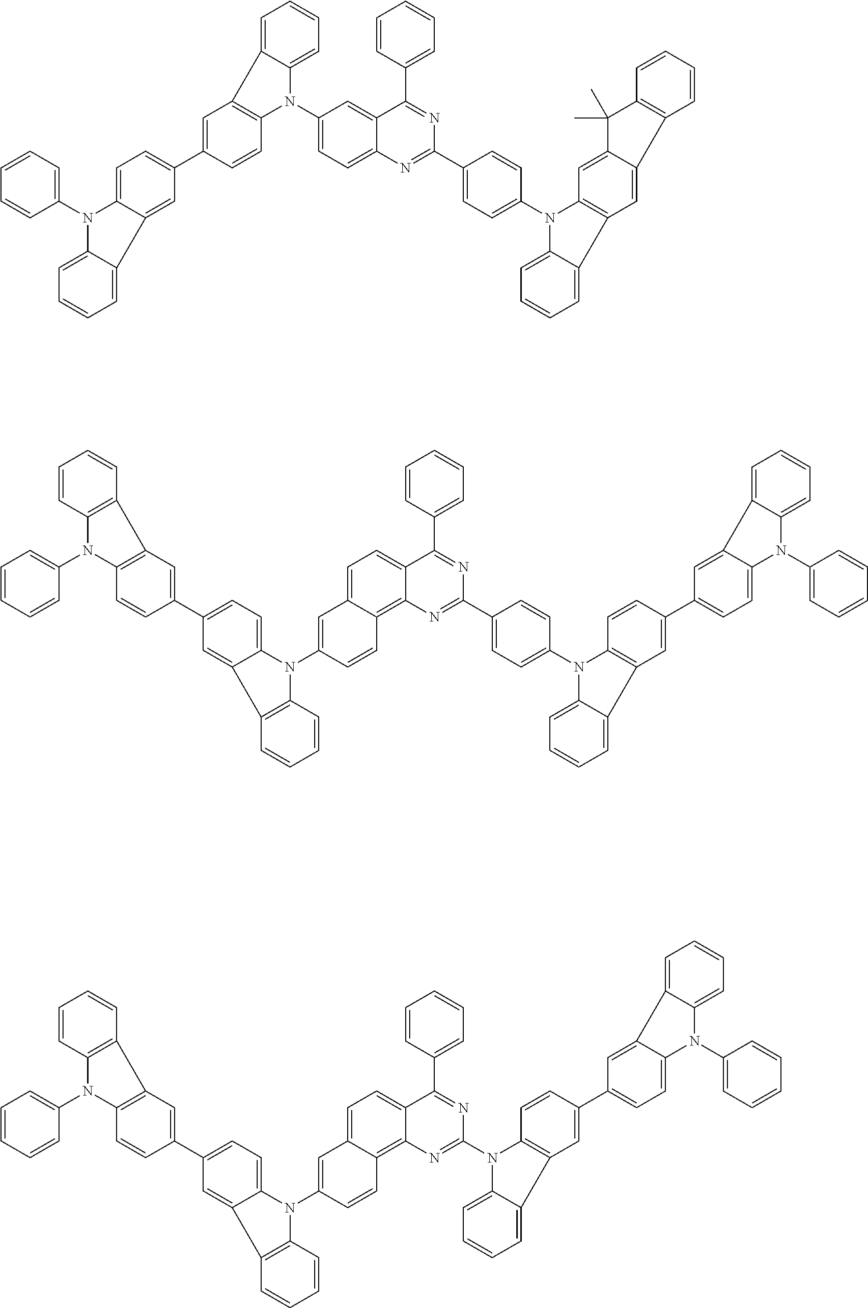 [SMILES: CC1(C)c2ccccc2-c2cc3c4ccccc4n(-c4ccc(-c5nc(-c6ccccc6)c6cc(-n7c8ccccc8c8cc(-c9ccc%10c(c9)c9ccccc9n%10-c9ccccc9)ccc87)ccc6n5)cc4)c3cc21 ; c1ccc(-c2nc(-c3ccc(-n4c5ccccc5c5cc(-c6ccc7c(c6)c6ccccc6n7-c6ccccc6)ccc54)cc3)nc3c2ccc2cc(-n4c5ccccc5c5cc(-c6ccc7c(c6)c6ccccc6n7-c6ccccc6)ccc54)ccc23)cc1 ; c1ccc(-c2nc(-n3c4ccccc4c4cc(-c5ccc6c(c5)c5ccccc5n6-c5ccccc5)ccc43)nc3c2ccc2cc(-n4c5ccccc5c5cc(-c6ccc7c(c6)c6ccccc6n7-c6ccccc6)ccc54)ccc23)cc1]

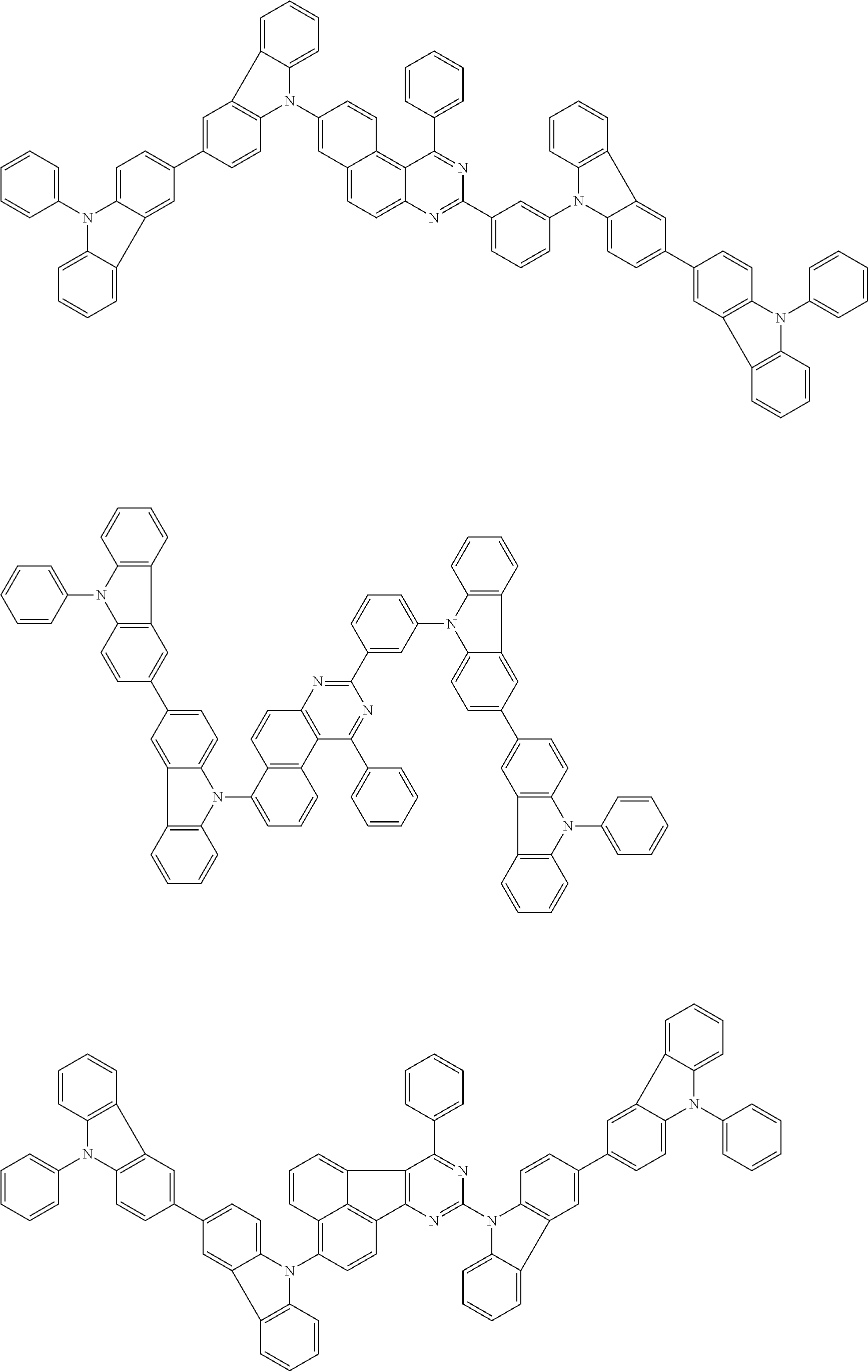 [SMILES: c1ccc(-c2nc(-c3cccc(-n4c5ccccc5c5cc(-c6ccc7c(c6)c6ccccc6n7-c6ccccc6)ccc54)c3)nc3ccc4cc(-n5c6ccccc6c6cc(-c7ccc8c(c7)c7ccccc7n8-c7ccccc7)ccc65)ccc4c23)cc1 ; c1ccc(-c2nc(-c3cccc(-n4c5ccccc5c5cc(-c6ccc7c(c6)c6ccccc6n7-c6ccccc6)ccc54)c3)nc3ccc4c(-n5c6ccccc6c6cc(-c7ccc8c(c7)c7ccccc7n8-c7ccccc7)ccc65)cccc4c23)cc1 ; c1ccc(-c2nc(-n3c4ccccc4c4cc(-c5ccc6c(c5)c5ccccc5n6-c5ccccc5)ccc43)nc3c2-c2cccc4c(-n5c6ccccc6c6cc(-c7ccc8c(c7)c7ccccc7n8-c7ccccc7)ccc65)ccc-3c24)cc1]

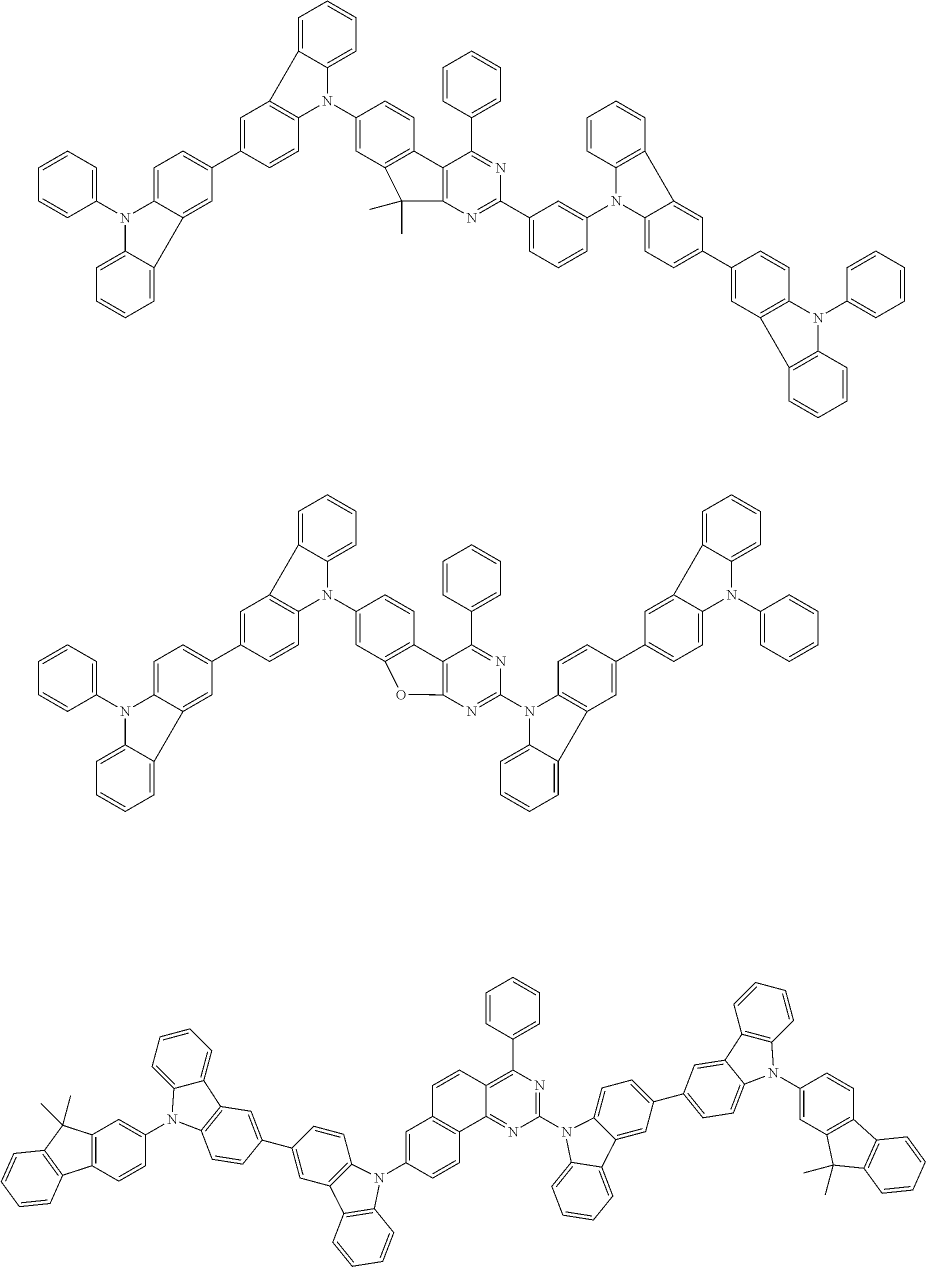

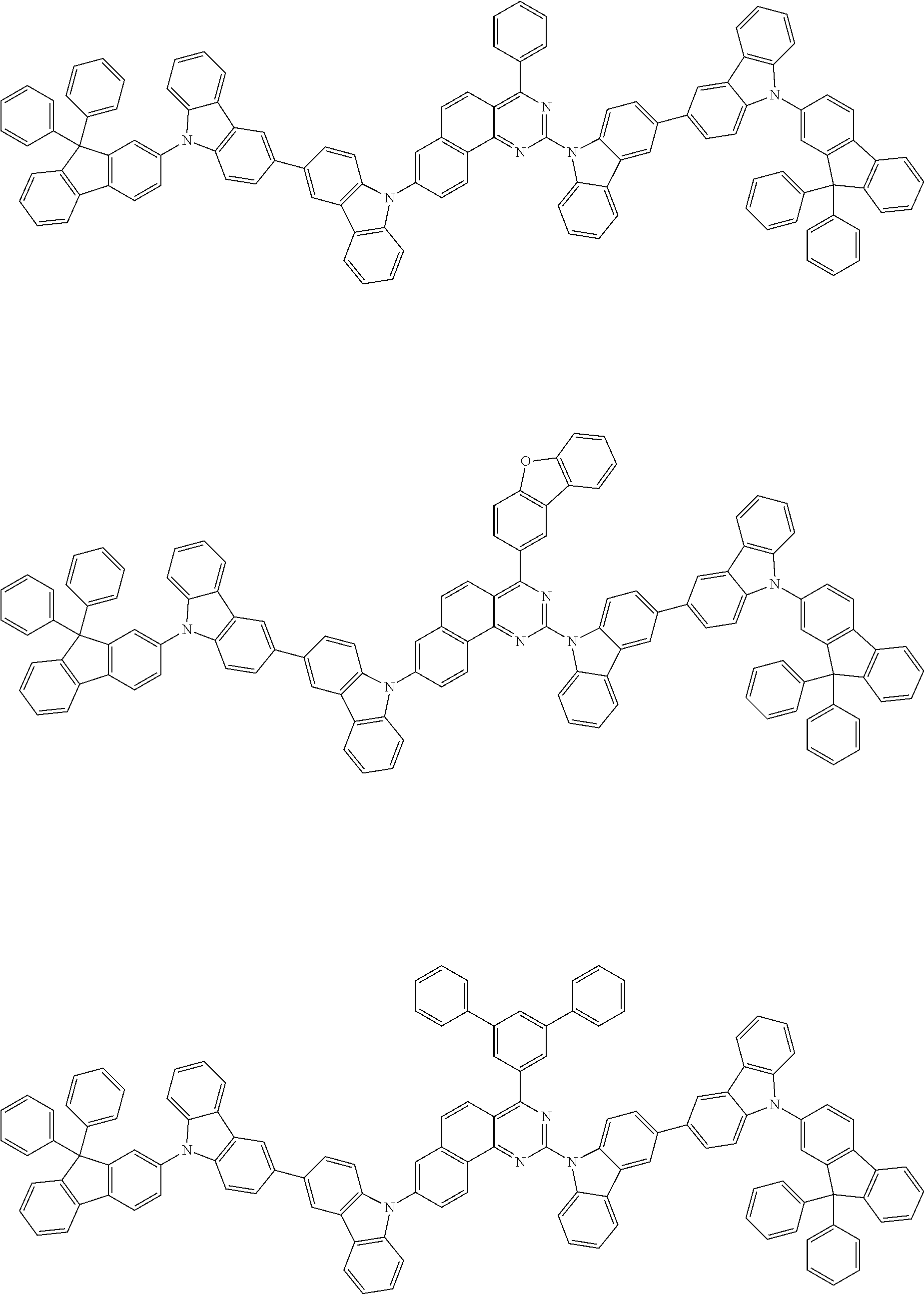

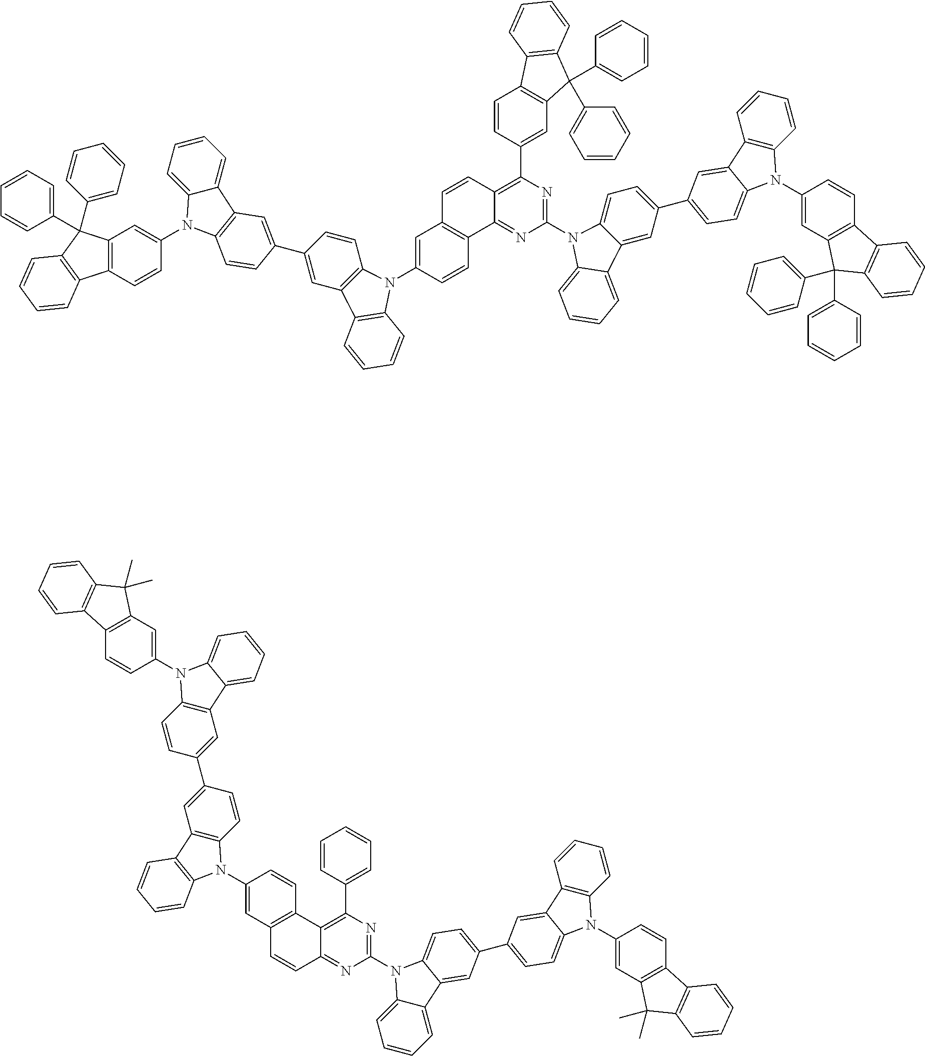 [SMILES: c1ccc(C2(c3ccccc3)c3ccccc3-c3ccc(-c4nc(-n5c6ccccc6c6cc(-c7ccc8c(c7)c7ccccc7n8-c7ccc8c(c7)C(c7ccccc7)(c7ccccc7)c7ccccc7-8)ccc65)nc5c4ccc4cc(-n6c7ccccc7c7cc(-c8ccc9c(c8)c8ccccc8n9-c8ccc9c(c8)C(c8ccccc8)(c8ccccc8)c8ccccc8-9)ccc76)ccc45)cc32)cc1 ; CC1(C)c2ccccc2-c2ccc(-n3c4ccccc4c4cc(-c5ccc6c(c5)c5ccccc5n6-c5ccc6c(ccc7nc(-n8c9ccccc9c9cc(-c%10ccc%11c(c%10)c%10ccccc%10n%11-c%10ccc%11c(c%10)C(C)(C)c%10ccccc%10-%11)ccc98)nc(-c8ccccc8)c76)c5)ccc43)cc21]

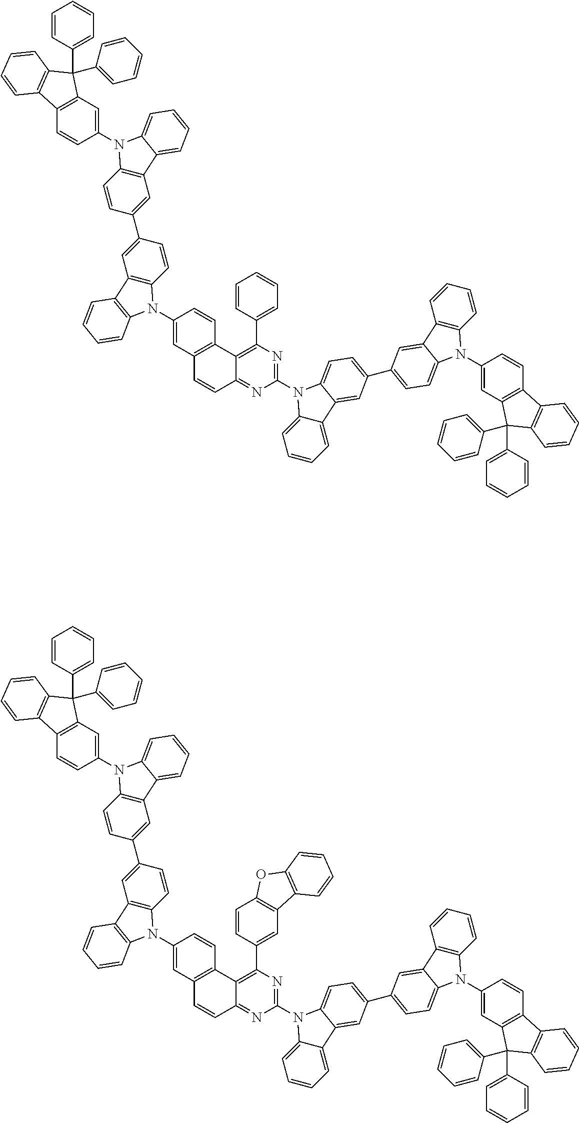 [SMILES: c1ccc(-c2nc(-n3c4ccccc4c4cc(-c5ccc6c(c5)c5ccccc5n6-c5ccc6c(c5)C(c5ccccc5)(c5ccccc5)c5ccccc5-6)ccc43)nc3ccc4cc(-n5c6ccccc6c6cc(-c7ccc8c(c7)c7ccccc7n8-c7ccc8c(c7)C(c7ccccc7)(c7ccccc7)c7ccccc7-8)ccc65)ccc4c23)cc1 ; c1ccc(C2(c3ccccc3)c3ccccc3-c3ccc(-n4c5ccccc5c5cc(-c6ccc7c(c6)c6ccccc6n7-c6ccc7c(ccc8nc(-n9c%10ccccc%10c%10cc(-c%11ccc%12c(c%11)c%11ccccc%11n%12-c%11ccc%12c(c%11)C(c%11ccccc%11)(c%11ccccc%11)c%11ccccc%11-%12)ccc%109)nc(-c9ccc%10oc%11ccccc%11c%10c9)c87)c6)ccc54)cc32)cc1]

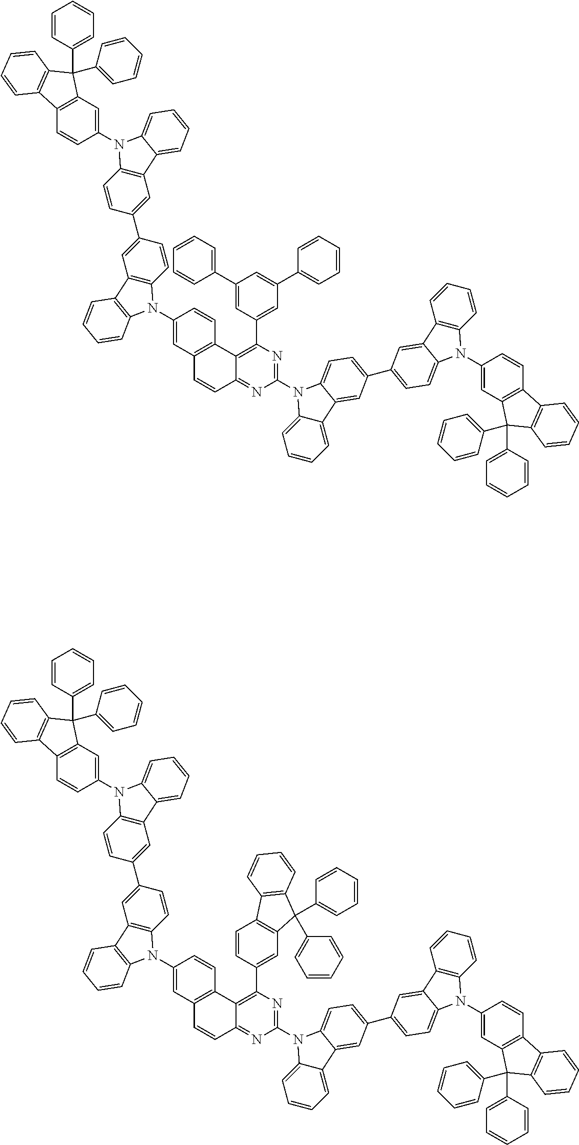 [SMILES: c1ccc(-c2cc(-c3ccccc3)cc(-c3nc(-n4c5ccccc5c5cc(-c6ccc7c(c6)c6ccccc6n7-c6ccc7c(c6)C(c6ccccc6)(c6ccccc6)c6ccccc6-7)ccc54)nc4ccc5cc(-n6c7ccccc7c7cc(-c8ccc9c(c8)c8ccccc8n9-c8ccc9c(c8)C(c8ccccc8)(c8ccccc8)c8ccccc8-9)ccc76)ccc5c34)c2)cc1 ; c1ccc(C2(c3ccccc3)c3ccccc3-c3ccc(-c4nc(-n5c6ccccc6c6cc(-c7ccc8c(c7)c7ccccc7n8-c7ccc8c(c7)C(c7ccccc7)(c7ccccc7)c7ccccc7-8)ccc65)nc5ccc6cc(-n7c8ccccc8c8cc(-c9ccc%10c(c9)c9ccccc9n%10-c9ccc%10c(c9)C(c9ccccc9)(c9ccccc9)c9ccccc9-%10)ccc87)ccc6c45)cc32)cc1]

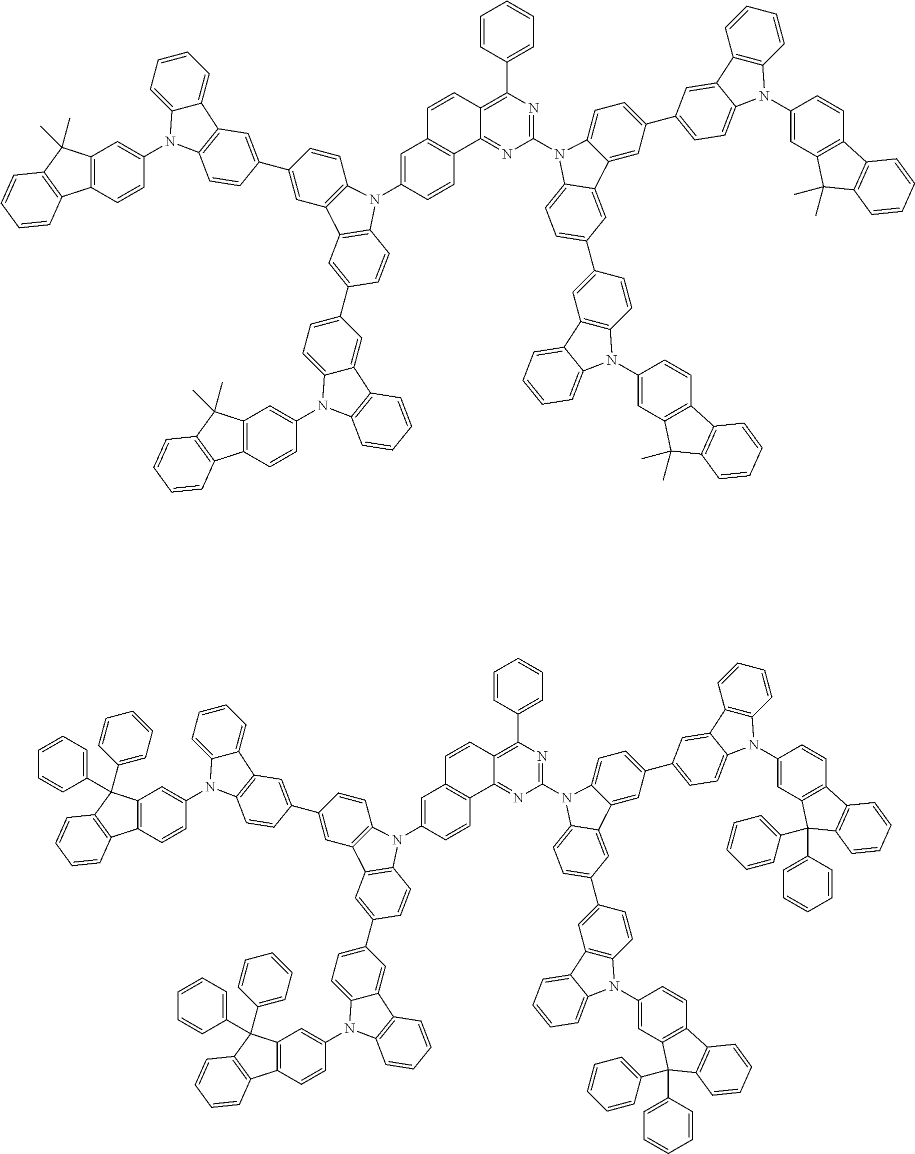

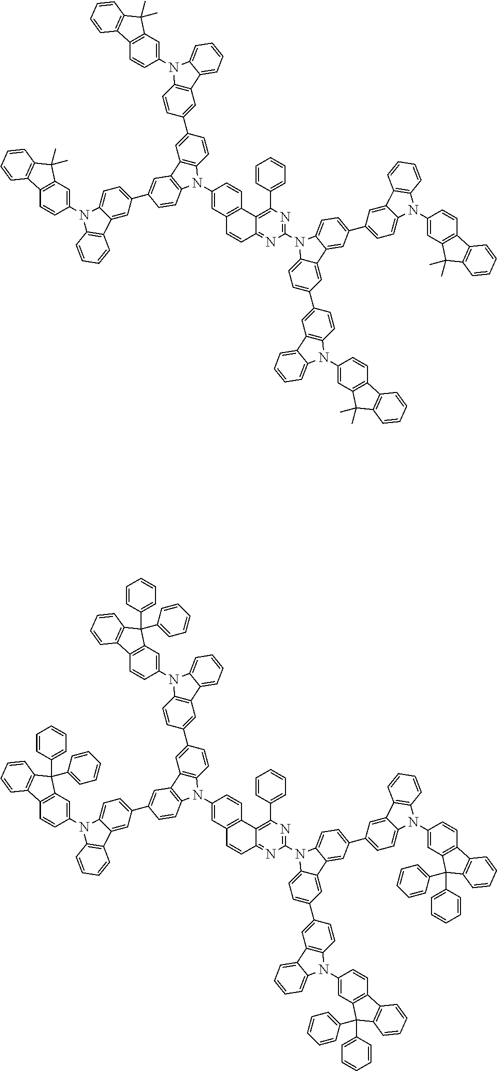

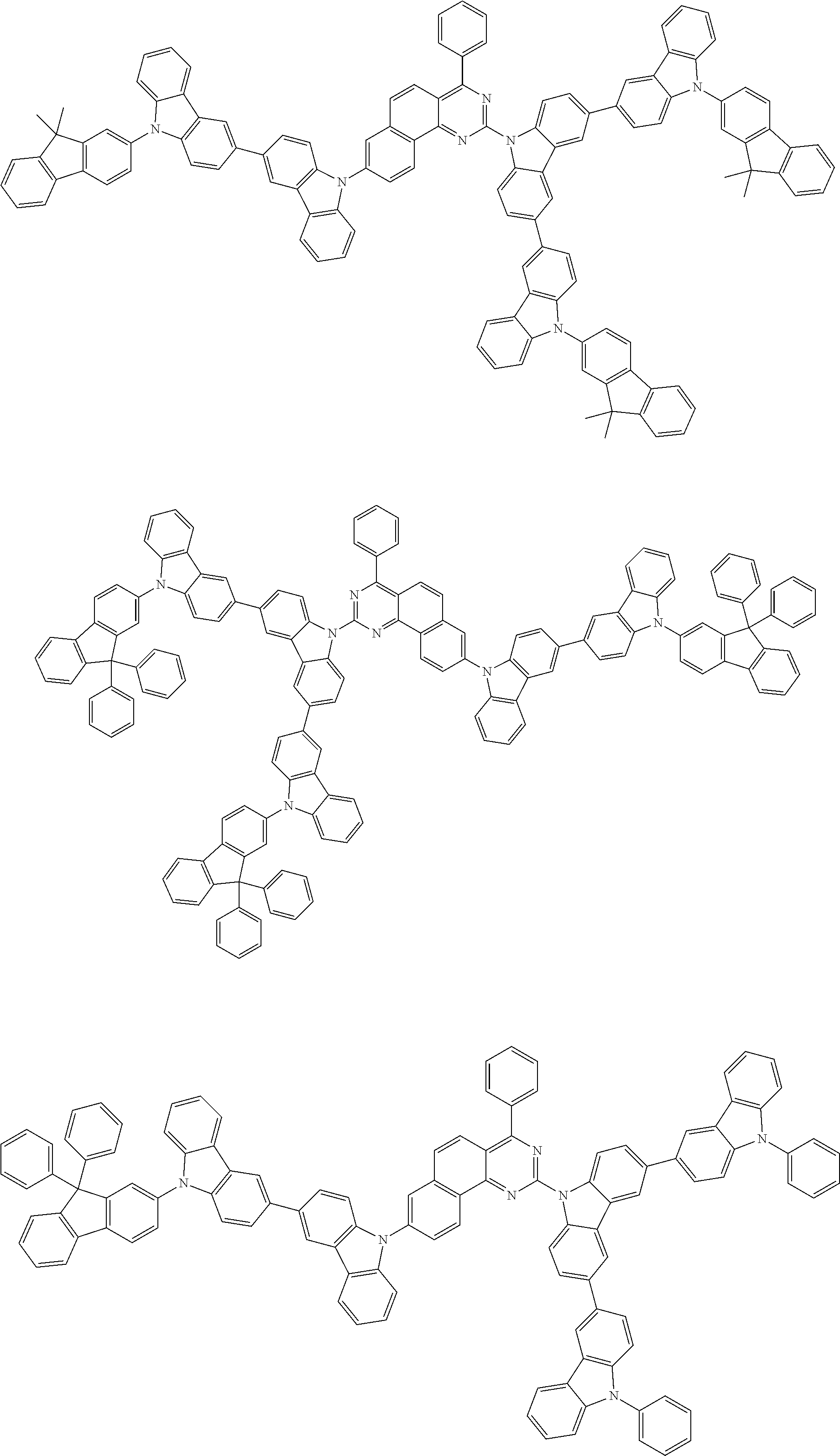

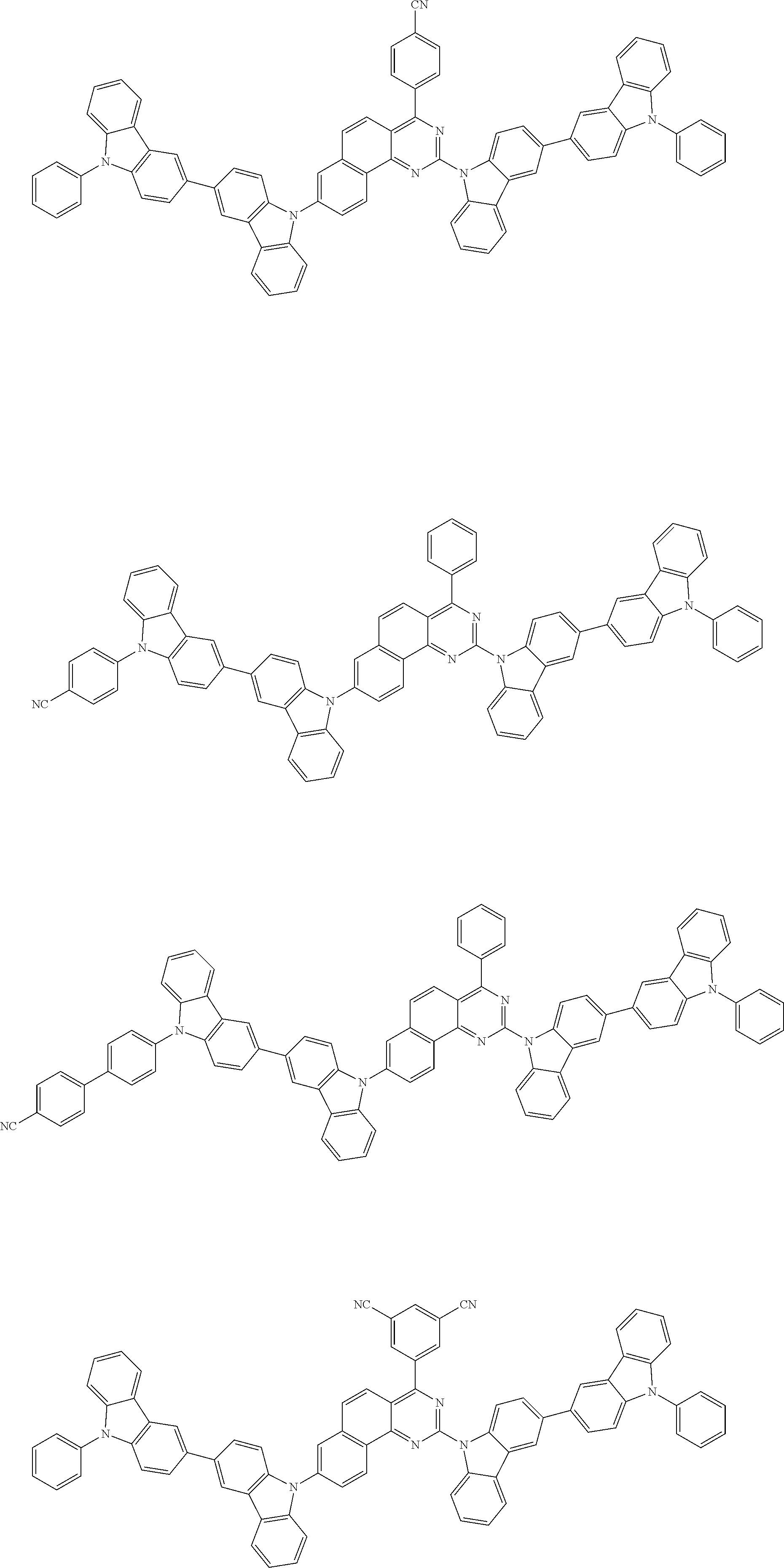

### Material for Organic Electroluminescence Device

The material for organic electroluminescence device (hereinafter also referred to as “material for organic EL device”) in an aspect of the invention will be described below.

The organic EL device comprises the compound (1). The compound (1) is useful as the material for producing an organic EL device.

The content of the compound (1) in the material for organic EL device may be 1% by mass or more, preferably 10% by mass or more, more preferably 50% by mass or more, still more preferably 80% by mass or more, and particularly preferably 90% by mass or more.

The material for organic EL device of the invention may be used, for example, in a light emitting layer of a fluorescent emission unit as a host material or a dopant material and in a light emitting layer of a phosphorescent emission unit as a host material. In this case, the light emitting layer comprises the material for organic EL device and a fluorescent emitting material or a phosphorescent emitting material.

In addition, in either a fluorescent emission unit or a phosphorescent emission unit, the material for organic EL device of the invention is also useful as a material for an anode-side organic thin film layer which is formed between an anode and a light emitting layer and a material for a cathode-side organic thin film layer which is formed between a cathode and a light emitting layer, i.e., also useful as a material for a hole transporting layer, a hole injecting layer, an electron transporting layer, an electron injecting layer, a hole blocking layer, and an electron blocking layer.

The “emission unit” referred to herein is the smallest unit for emitting light by the recombination of injected holes and injected electrons, which comprises one or more organic layers wherein at least one layer is a light emitting layer.

### Ink Composition

The ink composition according to an aspect of the invention will be described below.

The ink composition comprises a solvent and the compound (1) dissolved therein and is usable for forming an organic thin film layer of an organic EL device.

In addition to the compound (1), the ink composition may further comprise a hole transporting material, an electron transporting material, a light emitting material, an acceptor material, and an additive, such as a stabilizer.

The ink composition may contain an additive for controlling the viscosity and/or surface tension, for example, a thickener (a high molecular weight compound, etc.), a viscosity depressant (a low molecular weight compound, etc.) and a surfactant. In addition, an antioxidant not adversely affecting the performance of the organic EL device, for example, a phenol antioxidant and a phosphorus antioxidant, may be included so as to improve the storage stability.

The content of the compound (1) in the ink composition is preferably 0.1 to 15% by mass and more preferably 0.5 to 10% by mass

Examples of the high molecular weight compound usable as the thickener include an insulating resin, such as polystyrene, polycarbonate, polyarylate, polyester, polyamide, polyurethane, polysulfone, polymethyl methacrylate, polymethyl acrylate, cellulose, and a copolymer thereof; a photoconductive resin, such as poly-N-vinylcarbazole and polysilane; and an electroconductive resin, such as polythiophene and polypyrrole.

The solvent is preferably an organic solvent, for example, a chlorine-containing solvent, such as chloroform, chlorobenzene, chlorotoluene, chloroxylene, chloroanisole, dichloromethane, dichlorobenzene, dichlorotoluene, dichloroethane, trichloroethane, trichlorobenzene, trichloromethylbenzene, bromobenzene, dibromobenzene, and bromoanisole; an ether solvent, such as tetrahydrofuran, dioxane, dioxolane, oxazole, methylbenzoxazole, benzisooxazole, furan, furazan, benzofuran, and dihydrobenzofuran; an aromatic hydrocarbon solvent, such as ethylbenzene, diethylbenzene, triethylbenzene, trimethylbenzene, trimethoxybenzene, propylbenzene, isopropylbenzene, diisopropylbenzene, dibutylbenzene, amylbenzene, dihexylbenzene, cyclohexylbenzene, tetramethylbenzene, dodecylbenzene, benzonitrile, acetophenone, methylacetophenone, methoxyacetophenone, ethyl toluate, toluene, ethyltoluene, methoxytoluene, dimethoxytoluene, trimethoxytoluene, isopropyltoluene, xylene, butylxylene, isopropylxylene, anisole, ethylanisole, dimethylanisole, trimethylanisole, propylanisole, isopropylanisole, butylanisole, methylethylanisole, anethole, anisyl alcohol, methyl benzoate, ethyl benzoate, propyl benzoate, butyl benzoate, diphenyl ether, butyl phenyl ether, benzyl methyl ether, benzyl ethyl ether, methylenedioxybenzene, methylnaphthalene, tetrahydronaphthalene, aniline, methylaniline, ethylaniline, butylaniline, biphenyl, methylbiphenyl, and isopropylbiphenyl; an aliphatic hydrocarbon solvent, such as cyclohexane, methylcyclohexane, n-pentane, n-hexane, n-heptane, n-octane, n-nonane, n-decane, tetradecane, decalin, and isopropylcyclohexane; a ketone solvent, such as acetone, methyl ethyl ketone, cyclohexanone, and acetophenone; an ester solvent, such as ethyl acetate, butyl acetate, ethyl cellosolve acetate, methyl benzoate, and phenyl acetate; a polyhydric alcohol and its derivatives, such as ethylene glycol, ethylene glycol monobutyl ether, ethylene glycol monoethyl ether, ethylene glycol monomethyl ether, dimethoxyethane, propylene glycol, diethoxymethane, triethylene glycol monoethyl ether, glycerin, and 1,2-hexanediol; an alcoholic solvent, such as methanol, ethanol, propanol, isopropanol, and cyclohexanol; a sulfoxide solvent, such as dimethyl sulfoxide; and an amide solvent, such as N-methyl-2-pyrrolidone and N,N-dimethylformamide. These solvents may be used alone or in combination of two or more.

Of the above solvents, in view of solubility, uniform film formation, and viscosity, at least one of toluene, xylene, ethylbenzene, amylbenzene, anisole, 4-methoxytoluene, 2-methoxytoluene, 1,2-dimethoxybenzene, mesitylene, tetrahydronaphthalene, cyclohexylbenzene, 2,3-dihydrobenzofuran, cyclohexanone, methylcyclohexanone is preferably used.

The above solvents and a solvent represented by formula (a) having a boiling point of 110° C. or higher and a water solubility of 1% by mass or less at 20° C. are preferably used:

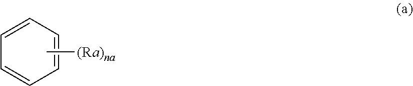

wherein Ra is independently a substituent having 1 to 20 carbon atoms and na is an integer of 0 to 6.

The film-forming solution (ink composition) preferably comprises the above solvents and the solvent represented by formula (a) having a boiling point of 110° C. or higher and a water solubility of 1% by mass or less at 20° C. The film-forming solution (ink composition) may further comprise, if necessary, a viscosity modifier, a surface tension regulator, a crosslinking initiator, or a crosslinking catalyst, which are preferably selected from those not adversely affecting the device performance even when remaining in the film or those being removable from the film in the film forming process.

### Organic Electroluminescence Device

The organic electroluminescence device (also referred to as “Organic EL device”) according to an aspect of the invention will be described below.

The organic EL device comprises one or more organic thin film layers between a cathode and an anode. The organic thin film layers comprise a light emitting layer and at least one layer of the organic thin film layers comprises the compound (1).

Examples of the organic thin film layer comprising the compound (1) include an anode-side organic thin film layer formed between an anode and a light emitting layer (hole transporting layer, hole injecting layer, etc.), a light emitting layer, a cathode-side organic thin film layer formed between a cathode and a light emitting layer (electron transporting layer, electron injecting layer, etc.), a space layer, and a blocking layer, although not limited thereto. The compound (1) may be used in any of the above layers, for example, used in a light emitting layer of a fluorescent emission unit as a host material or a dopant material, in a light emitting layer of a phosphorescent emission unit as a host material, or in a hole transporting layer, an electron transporting layer, etc. of an emission unit.

In an embodiment of the invention, the organic EL device may be any of a single color emitting fluorescent or phosphorescent device, a white-emitting fluorescent-phosphorescent hybrid device, a simple type emitting device having a single emission unit, and a tandem type emitting device having two or more emission units, with the phosphorescent device being preferred. The “emission unit” referred to herein is the smallest unit for emitting light by the recombination of injected holes and injected electrons, which comprises one or more organic layers wherein at least one layer is a light emitting layer.

Representative device structures of the simple-type organic EL device are shown below.

### (1) Anode/Emission Unit/Cathode

The emission unit may be a laminate comprising two or more phosphorescent emitting layers and two or more phosphorescent emitting layers. A space layer may be disposed between light emitting layers to prevent the diffusion of excitons generated in a phosphorescent emitting layer into a phosphorescent emitting layer. Representative layered structures of the emission unit are shown below:

(a) Hole transporting layer/Light emitting layer (/Electron transporting layer);
(b) Hole transporting layer/First phosphorescent emitting layer/Second phosphorescent emitting layer (/Electron transporting layer);
(c) Hole transporting layer/Phosphorescent emitting layer/Space layer/Phosphorescent emitting layer (/Electron transporting layer);
(d) Hole transporting layer/First phosphorescent emitting layer/Second phosphorescent emitting layer/Space layer/Phosphorescent emitting layer (/Electron transporting layer);
(e) Hole transporting layer/First phosphorescent emitting layer/Space layer/Second phosphorescent emitting layer/Space layer/Phosphorescent emitting layer (/Electron transporting layer);
(f) Hole transporting layer/Phosphorescent emitting layer/Space layer/First phosphorescent emitting layer/Second phosphorescent emitting layer (/Electron transporting layer);
(g) Hole transporting layer/Electron blocking layer/Light emitting layer (/Electron transporting layer);
(h) Hole transporting layer/Light emitting layer/Hole blocking layer (/Electron transporting layer); and
(i) Hole transporting layer/Phosphorescent emitting layer/Triplet blocking layer (/Electron transporting layer).

The emission color of a phosphorescent emitting layer and that of a phosphorescent emitting layer may be different. For example, the layered structure of the laminated light emitting layer (d) may be Hole transporting layer/First phosphorescent emitting layer (red)/Second phosphorescent emitting layer (green)/Space layer/Phosphorescent emitting layer (blue)/Electron transporting layer.

An electron blocking layer may be disposed between a light emitting layer and a hole transporting layer or between a light emitting layer and a space layer, if necessary. Also, a hole blocking layer may be disposed between a light emitting layer and an electron transporting layer, if necessary. With such an electron blocking layer or a hole blocking layer, electrons and holes are confined in a light emitting layer to enhance the charge recombination in the light emitting layer, thereby improving the lifetime.

A representative device structure of the tandem-type organic EL device is shown below:

### (2) Anode/First Emission Unit/Intermediate Layer/Second Emission Unit/Cathode.

The layered structures of the first emission unit and the second emission unit may be independently selected from those described above with respect to the emission unit.

Generally, the intermediate layer is also called an intermediate electrode, an intermediate conductive layer, a charge generation layer, an electron withdrawing layer, a connecting layer, or an intermediate insulating layer. The intermediate layer may be formed by a known material capable of supplying electrons to the first emission unit and holes to the second emission unit.

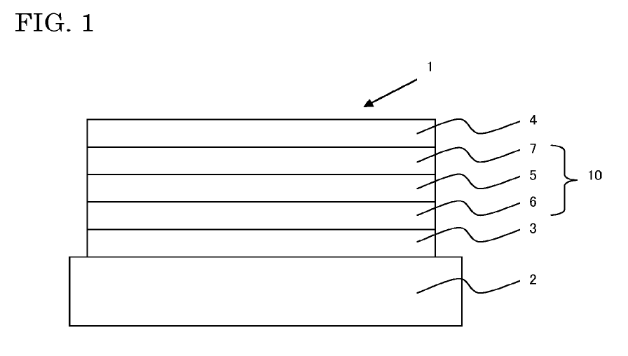

A schematic structure of an example of the organic EL device is shown in FIG. 1, wherein the organic EL device 1 comprises a substrate 2, an anode 3, a cathode 4, and an emission unit 10 disposed between the anode 3 and the cathode 4. The emission unit 10 comprises a light emitting layer 5 which comprises at least one phosphorescent emitting layer comprising a phosphorescent host and a phosphorescent dopant (phosphorescent emitting material). A hole injecting/transporting layer (an anode-side organic thin film layer) 6 may be disposed between the light emitting layer 5 and the anode 3, and an electron injecting/transporting layer (a cathode-side thin film layer) 7 may be disposed between the light emitting layer 5 and the cathode 4. An electron blocking layer may be disposed on the anode 3 side of the light emitting layer 5, and a hole blocking layer may be disposed on the cathode 4 side of the light emitting layer 5. With these blocking layers, electrons and holes are confined in the light emitting layer 5 to enhance the exciton generation in the light emitting layer 5.

In the present invention, a host is referred to as a fluorescent host when combinedly used with a fluorescent dopant (fluorescent emitting material) and as a phosphorescent host when combinedly used with a phosphorescent dopant. Therefore, the fluorescent host and the phosphorescent host are not distinguished from each other merely by the difference in their molecular structures. Namely, in the present invention, the term “phosphorescent host” means a material for constituting a phosphorescent emitting layer containing a phosphorescent dopant and does not necessarily mean a material that cannot be used in a fluorescent emitting layer. The same applies to the fluorescent host.

### Substrate

The organic EL device is formed on a light-transmissive substrate. The light-transmissive substrate serves as a support for the organic EL device and is preferably a flat substrate having a transmittance of 50% or more to 400 to 700 nm visible light. Examples of the substrate include a glass plate and a polymer plate. The glass plate may include a plate made of soda-lime glass, barium-strontium-containing glass, lead glass, aluminosilicate glass, borosilicate glass, barium borosilicate glass, or quartz. The polymer plate may include a plate made of polycarbonate, acryl, polyethylene terephthalate, polyether sulfide, or polysulfone.

### Anode

The anode of an organic EL device injects holes to a hole transporting layer or a light emitting layer, and a material having a work function of 4.5 eV or more is effective for the anode. Examples of the material for anode include indium tin oxide alloy (ITO), tin oxide (NESA), indium zinc oxide, gold, silver, platinum, and cupper. The anode is formed by making the material for anode into a thin film by a method, such as a vapor deposition method or a sputtering method. When getting the light emitted from a light emitting layer through the anode, the transmittance of anode to visible light is preferably 10% or more. The sheet resistance of anode is preferably several hundreds Ω/or less. The film thickness of anode depends upon the kind of material and generally 10 nm to 1 μm, preferably 10 to 200 nm.

### Cathode

The cathode injects electrons to an electron injecting layer, an electron transporting layer or a light emitting layer and is formed preferably by a material having a small work function. Examples of the material for cathode include, but not limited to, indium, aluminum, magnesium, magnesium-indium alloy, magnesium-aluminum alloy, aluminum-lithium alloy, aluminum-scandium-lithium alloy, and magnesium-silver alloy. Like the anode, the cathode is formed by making the material for cathode into a thin film by a method, such as a vapor deposition method and a sputtering method. The emitted light may be taken through the cathode, if necessary.

### Light Emitting Layer

The light emitting layer is an organic layer having a light emitting function and comprises a host material and a dopant material (highly emitting material) when a doping system is employed. The dopant material may be a fluorescent emitting material or a phosphorescent emitting material. The fluorescent emitting material is a compound capable of emitting light from a singlet excited state, and the phosphorescent emitting compound is a compound capable of emitting light from a triplet exciting state. The host material mainly promotes the recombination of electrons and holes and confines the excitons within the light emitting layer. The dopant material lets the excitons generated by the recombination emit light efficiently.

In a phosphorescent device, the major function of the host material is to confine the excitons generated on the dopant within a light emitting layer.

To control the carrier balance in a light emitting layer, the light emitting layer may be made into a double host (host/co-host) layer, for example, by combinedly using an electron transporting host material and a hole transporting host material.

The light emitting layer may be also made into a double dopant layer, in which two or more kinds of dopant materials having a high quantum yield are combinedly used and each dopant material emits light with its own color. For example, a yellow emission can be obtained by a light emitting layer which is formed by co-depositing a host material, a red-emitting dopant material, and a green-emitting dopant material.

In a laminate of two or more light emitting layers, electrons and holes can be accumulated in the interface between the light emitting layers, and therefore, the recombination region is localized in the interface between the light emitting layers. With this structure, the quantum efficiency can be enhanced.

The easiness of hole injection to a light emitting layer and the easiness of electron injection to a light emitting layer may be different from each other. Also, the hole transporting ability and the electron transporting ability each being expressed by mobility of holes and electrons in a light emitting layer may be different from each other.

The phosphorescent dopant material (phosphorescent emitting material) to be used in a light emitting layer is a compound which emits light by releasing the energy of excited triplet state and preferably a organometallic complex comprising at least one metal selected from Ir, Pt, Os, Au, Cu, Re, and Ru and a ligand, although not particularly limited thereto as long as emitting light by releasing the energy of excited triplet state. For example, a metal complex, such as an iridium complex, an osmium complex, and a platinum complex, is used as a blue-emitting phosphorescent dopant material. Examples thereof include bis[2-(4′,6′-difluorophenyl)pyridinato-N,C2′]iridium(III) tetrakis(1-pyrazolyl)borato (FIr₆), bis[2-(4′,6′-difluorophenyl)pyridinato-N,C2′]iridium(III) picolinato (FIrpic), bis[2-(3′,5′-bistrifluoromethylphenyl)pyridinato-N,C2′]iridium(III) picolinato (Ir(CF₃ppy)₂(pic)), and bis[2-(4′,6′-difluorophenyl)pyridinato-N,C2′]iridium(III) acetylacetonato (FIracac).

An iridium complex is used as a green-emitting phosphorescent dopant material. Examples thereof include tris(2-phenylpyridinato-N,C2′)iridium(III) (Ir(ppy)₃), bis(2-phenylpyridinato-N,C2′)iridium(III) acetylacetonato (Ir(ppy)₂(acac)), bis(1,2-diphenyl-1H-benzimidazolato)iridium(III) acetylacetonato (Ir(pbi)₂(acac)), and bis(benzo[h]quinolinato)iridium (III) acetylacetonato (Ir(bzq)₂(acac)).

A metal complex, such as an iridium complex, a platinum complex, a terbium complex, and a europium complex, is used as a red-emitting phosphorescent dopant material. Examples thereof include an organometallic complex, such as bis[2-(2′-benzo[4,5-α ]thienyl)pyridinato-N,C3′]iridium(III) acetylacetonato (Ir(btp)₂(acac)), bis(1-phenylisoquinolinato-N,C2′)iridium(III) acetylacetonato (Ir(piq)₂(acac)), (acetylacetonato)bis[2,3-bis(4-fluorophenyl)quinoxalinato]iridium(III) (Ir(Fdpq)₂(acac)), and 2,3,7,8,12,13,17,18-octaethyl-21H,23H-porphyrin platinum(II) (PtOEP).

A rare earth metal complex, such as tris(acetylacetonato) (monophenanthroline)terbium(III) (Tb(acac)s(Phen)), tris(1,3-diphenyl-1,3-propanedionato)(monophenanthroline)europium(III) (Eu(DBM)s(Phen)), and tris[1-(2-thenoyl)-3,3,3-trifluoroacetonato](monophenanthroline)europium(III) (Eu(TTA)₃(Phen)), emits light from the rare earth metal ion (electron transition between different multiple states), and therefore, usable as a phosphorescent emitting compound.

The ligand is preferably ortho-metallated. In view of obtaining a high phosphorescent quantum yield and further improving the external quantum efficiency of luminescent device, a metal complex comprising a metal selected from Ir, Os, and Pt is preferred, with a metal complex, such as an iridium complex, an osmium complex and a platinum complex, particularly an ortho-metallated complex being more preferred, an iridium complex and a platinum complex being still more preferred, and an ortho-metallated iridium complex being particularly preferred.

The content of the phosphorescent dopant material in a light emitting layer is not particularly limited and selected according to the use of the device, and preferably 0.1 to 70% by mass, and more preferably 1 to 30% by mass. If being 0.1% by mass or more, the amount of light emission is sufficient. If being 70% by mass or less, the concentration quenching can be avoided.

Preferred examples of the organometallic complex for the phosphorescent dopant material are shown below.

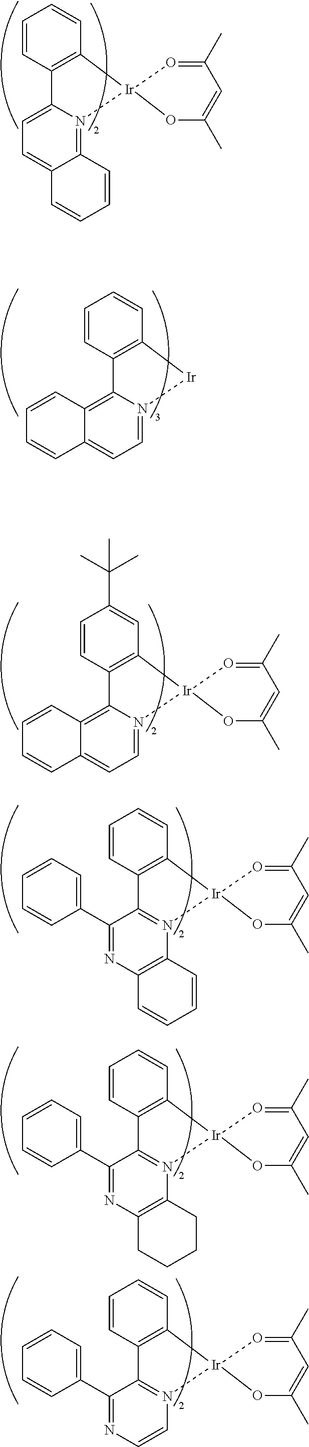

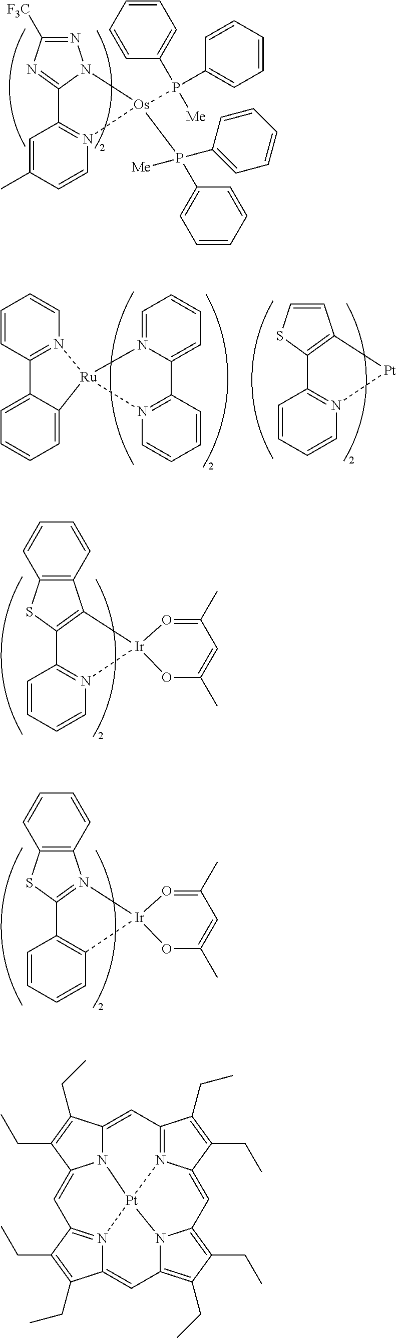

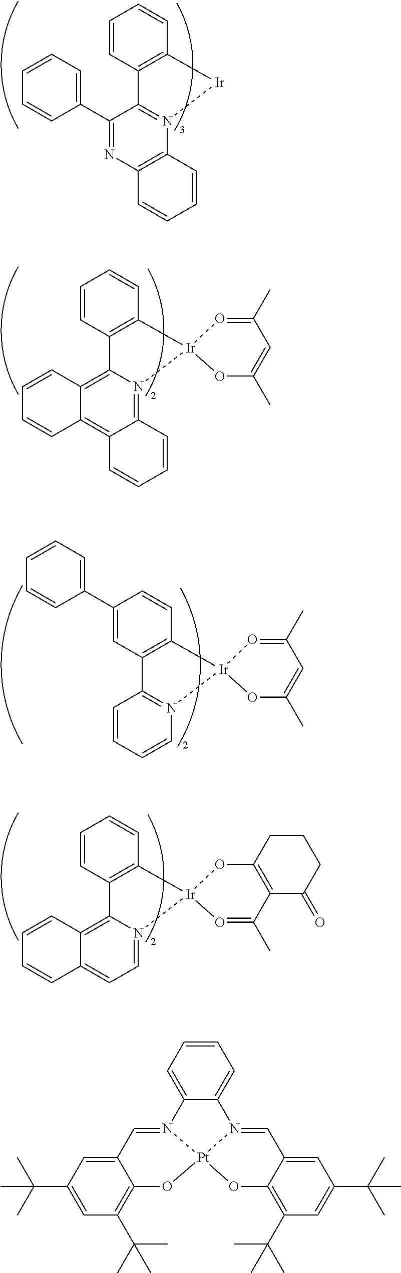

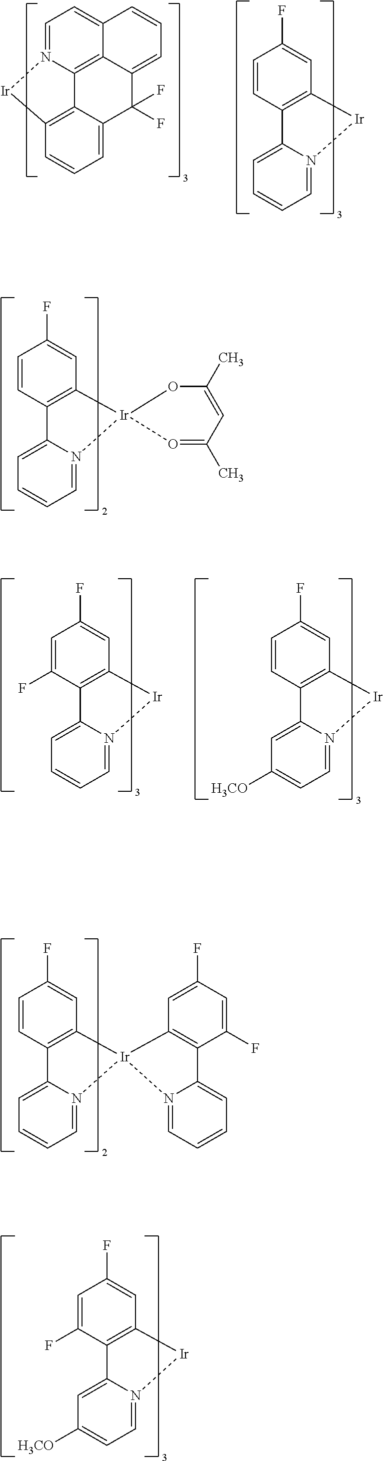

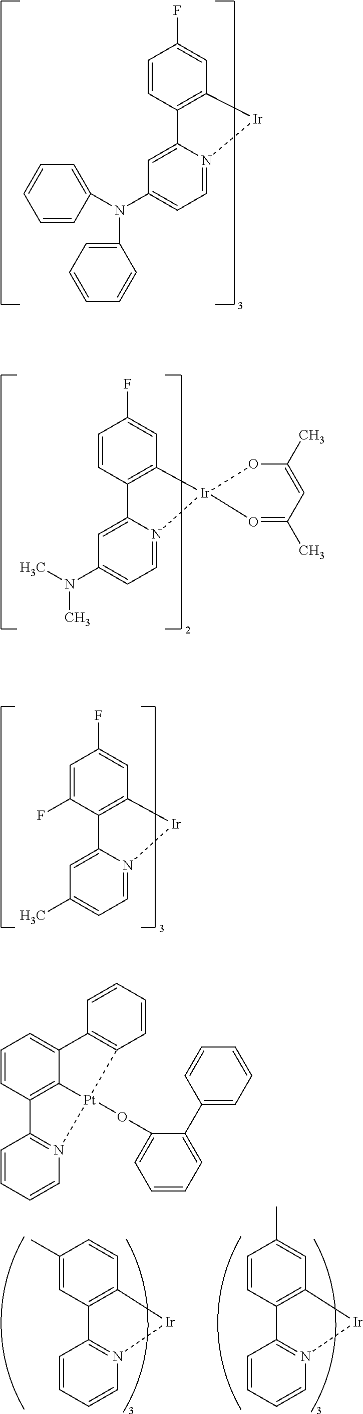

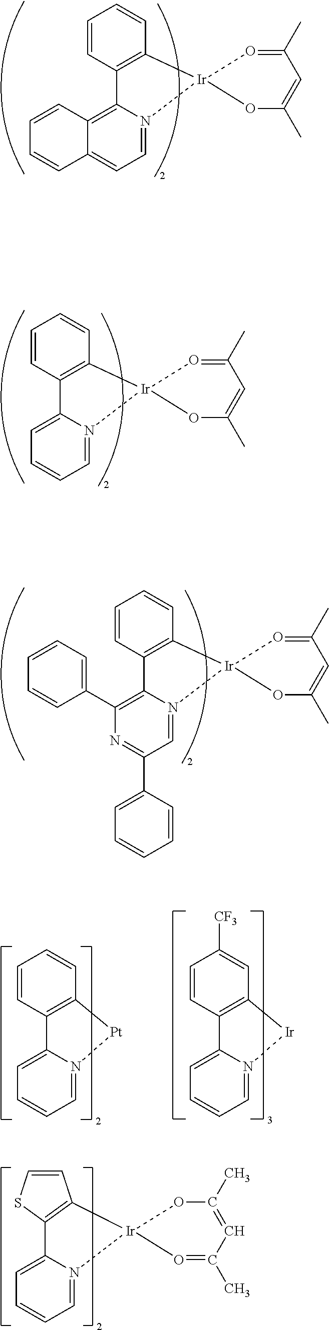

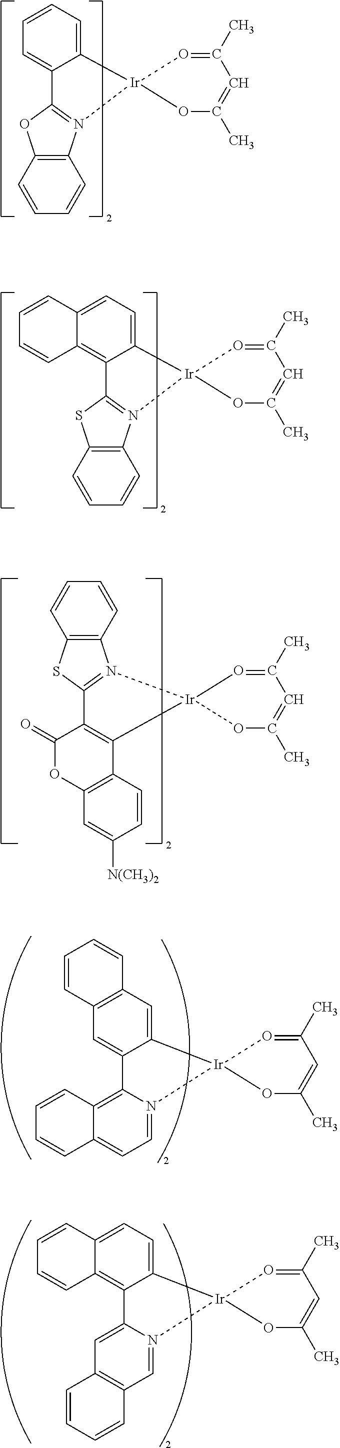

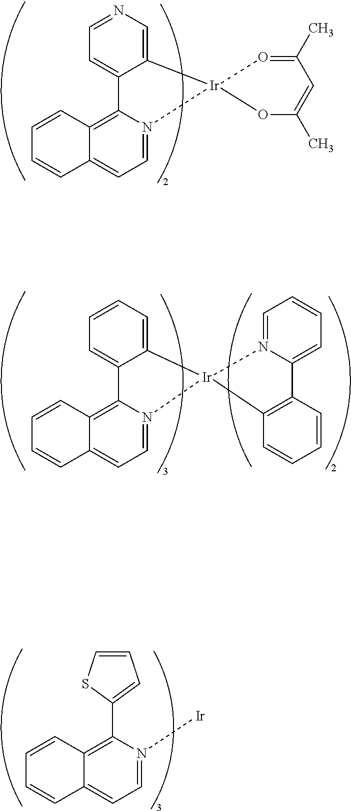

A complex represented by formula (X) or (Y) is preferably used as the phosphorescent dopant material:

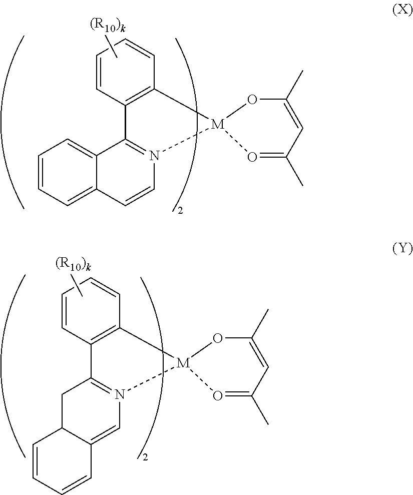

wherein R₁₀ represents a hydrogen atom or a substituent, k represents an integer of 1 to 4, and M represents Ir, Os, or Pt.

Examples of the substituent as R₁₀ are the same as those mentioned above with respect to R₀ to R₈, etc. of formula (1).

In an embodiment of the invention, the organic EL device may comprise a light emitting layer comprising a fluorescent material, i.e., a fluorescent emitting layer. The fluorescent emitting layer may be formed from a known fluorescent emitting material.

In an embodiment of the invention, for example, a pyrene derivative, a styrylamine derivative, a chrysene derivative, a fluoranthene derivative, a fluorene derivative, a diamine derivative, and a triarylamine derivative are usable as a blue fluorescent emitting material. Examples thereof include N,N′-bis[4-(9H-carbazole-9-yl)phenyl]-N,N′-diphenylstilbene-4,4′-diamine (YGA2S), 4-(9H-carbazole-9-yl)-4′-(10-phenyl-9-anthryl)triphenylamine (YGAPA), and 4-(10-phenyl-9-anthryl)-4′-(9-phenyl-9H-carbazole-3-yl)triphenylamine (PCBAPA).

An aromatic amine derivative is usable as a green fluorescent emitting material. Examples thereof include N-(9,10-diphenyl-2-anthryl)-N,9-diphenyl-9H-carbazole-3-amine (2PCAPA), N-[9,10-bis(1, 1′-biphenyl-2-yl)-2-anthryl]-N,9-diphenyl-9H-carbazole-3-amine (2PCABPhA), N-(9,10-diphenyl-2-anthryl)-N,N′,N′-triphenyl-1,4-phenylenediamine (2DPAPA), N-[9,10-bis(1,1′-biphenyl-2-yl)-2-anthryl]-N,N′,N′-triphenyl-1,4-phenylenediamine (2DPABPhA), N-[9,10-bis(1, 1′-biphenyl-2-yl)]-N-[4-(9H-carbazole-9-yl)phenyl]-N-phenylanthracene-2-amine (2YGABPhA), and N,N,9-triphenylanthracene-9-amine (DPhAPhA).

A tetracene derivative and a diamine derivative are usable as a red fluorescent emitting material. Examples thereof include N,N,N′,N′-tetrakis(4-methylphenyl)tetracene-5,11-diamine (p-mPhTD) and 7,14-diphenyl-N,N,N′,N′-tetrakis(4-methylphenyl)acenaphtho[1,2-a]fluoranthene-3,10-diamine (p-mPhAFD).

In another embodiment of the invention, at least one material selected from an anthracene derivative, a fluoranthene derivative, a styrylamine derivative, and an arylamine derivative is preferably used as the fluorescent emitting material, with the anthracene derivative and the arylamine derivative being more preferred. In particular, the anthracene derivative is preferably used as a host material and the arylamine derivative is preferably used as a dopant. The materials described in WO 2010/134350 and WO 2010/134352 are preferably used. The compound (1) and the material for organic EL devices may be used in a fluorescent emitting layer as a fluorescent emitting material or a host material.

The highly light-emitting material (dopant material) may be dispersed in another material (host material). The host material may be selected from various kinds of materials and is preferably a material having a lowest unoccupied molecular orbital level (LUMO level) higher than that of the dopant material and a highest occupied molecular orbital level (HOMO level) lower than that of the dopant material.

In an embodiment of the invention, the material may include (1) a metal complex, such as an aluminum complex, a beryllium complex, and a zinc complex; (2) a heterocyclic compound, such as an oxadiazole derivative, a benzimidazole derivative, and a phenanthroline derivative; (3) a fused aromatic compound, such as a carbazole derivative, an anthracene derivative, a phenanthrene derivative, a pyrene derivative, and a chrysene derivative; and (4) an aromatic amine compound, such as a triarylamine derivative and a fused polycyclic aromatic amine derivative. Examples thereof include:

a metal complex, such as tris(8-quinolinolato)aluminum(III) (Alq), tris(4-methyl-8-quinolinolato)aluminum(III) (Almq₃), bis(10-hydroxybenzo[h]quinolinato)beryllium(II) (BeBq₂), bis(2-methyl-8-quinolinolato)(4-phenylphenolato)aluminum(III) (BAlq), bis(8-quinolinolato)zinc(II) (Znq), bis[2-(2-benzoxazolyl)phenolato]zinc(II) (ZnPBO), and bis[2-(2-benzothiazolyl)phenolato]zinc(II) (ZnBTZ);

a heterocyclic compound, such as 2-(4-biphenylyl)-5-(4-tert-butylphenyl)-1,3,4-oxadiazole (PBD), 1,3-bis[5-(p-tert-butylphenyl)-1,3,4-oxadiazole-2-yl]benzene (OXD-7), 3-(4-biphenylyl)-4-phenyl-5-(4-tert-butylphenyl)-1,2,4-triazole (TAZ), 2,2′,2″-(1,3,5-benzenetriyl)tris(1-phenyl-1H-benzimidazole) (TPBI), bathophenanthroline (BPhen), and bathocuproin (BCP);

a fused aromatic compound, such as 9-[4-(10-phenyl-9-anthryl)phenyl]-9H-carbazole (CzPA), 3,6-diphenyl-9-[4-(10-phenyl-9-anthryl)phenyl]-9H-carbazole (DPCzPA), 9,10-bis(3,5-diphenylphenyl)anthracene (DPPA), 9,10-di(2-naphthyl)anthracene (DNA), 2-tert-butyl-9,10-di(2-naphthyl)anthracene (t-BuDNA), 9,9′-bianthryl (BANT), 9,9′-(stilbene-3,3′-diyl)diphenanthrene (DPNS), 9,9′-(stilbene-4,4′-diyl)diphenanthrene (DPNS2), 3,3′,3″-(benzene-1,3,5-triyl)tripyrene (TPB3), 9,10-diphenylanthracene (DPAnth), and 6,12-dimethoxy-5,11-diphenylchrysene; and

an aromatic amine compound, such as N,N-diphenyl-9-[4-(10-phenyl-9-anthryl)phenyl]-9H-carbazole-3-amine (CzA1PA), 4-(10-phenyl-9-anthryl)triphenylamine (DPhPA), N,9-diphenyl-N-[4-(10-phenyl-9-anthryl)phenyl]-9H-carbazole-3-amine (PCAPA), N,9-diphenyl-N-{4-[4-(10-phenyl-9-anthryl)phenyl]phenyl}-9H-carbazole-3-amine (PCAPBA), N-(9,10-diphenyl-2-anthryl)-N,9-diphenyl-9H-carbazole-3-amine (2PCAPA), NPB (α-NPD), TPD, DFLDPBi, and BSPB.

The phosphorescent host is a compound which confines the triplet energy of the phosphorescent dopant efficiently within a light emitting layer to cause the phosphorescent dopant to emit light efficiently. Although the compound (1) and the material for organic EL device comprising the compound (1) are useful as a phosphorescent host, a compound other than the compound (1) may be used as the phosphorescent host according to the use of the device. The use of the compound (1) and the material for organic EL devices is not limited to the phosphorescent host.

The compound (1) and a compound other than it may be combinedly used in the same light emitting layer as the phosphorescent host materials. Alternatively, the compound (1) may be used in one of light emitting layers as a phosphorescent host material and a compound other than it may be used in another of the light emitting layers as a phosphorescent host material. The compound (1) may be used in an organic layer other than the light emitting layer. In this case, a compound other than the compound (1) may be used as a phosphorescent host of the light emitting layer.

Examples of the compound other than the compound (1) which is suitable as a phosphorescent host include a carbazole derivative, a triazole derivative, a oxazole derivative, an oxadiazole derivative, an imidazole derivative, a polyarylalkane derivative, a pyrazoline derivative, a pyrazolone derivative, a phenylenediamine derivative, an arylamine derivative, an amino-substituted chalcone derivative, a styrylanthracene derivative, a fluorenone derivative, a hydrazone derivative, a stilbene derivative, a silazane derivative, an aromatic tertiary amine compound, a styrylamine compound, an aromatic methylidene compound, a porphyrin compound, an anthraquinodimethane derivative, an anthrone derivative, a diphenylquinone derivative, a thiopyran dioxide derivative, a carbodiimide derivative, a fluorenylidenemethane derivative, a distyrylpyrazine derivative, a tetracarboxylic anhydride of fused ring such as naphthalene and perylene, a phthalocyanine derivative, a metal complex of 8-quinolinol derivative, metal phthalocyanine, metal complexes having a ligand such as benzoxazole and benzothiazole, an electroconductive oligomer, such as a polysilane compound, a poly(N-vinylcarbazole) derivative, an aniline copolymer, thiophene oligomer, and a polythiophene, and a polymer such as a polythiophene derivative, a polyphenylene derivative, a polyphenylenevinylene derivative, and a polyfluorene derivative. These phosphorescent hosts may be used alone or in combination of two or more. Examples thereof are shown below:

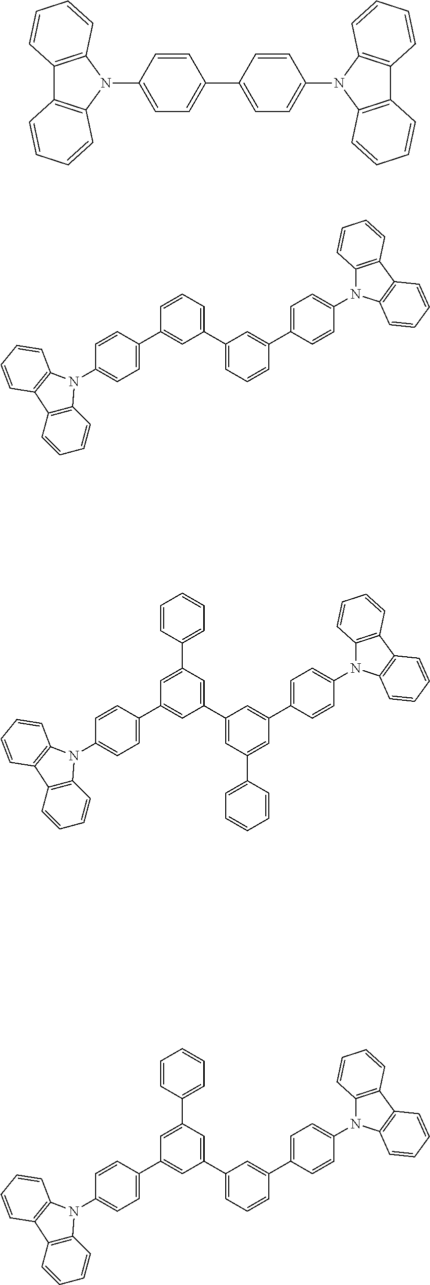 [SMILES: c1ccc2c(c1)c1ccccc1n2-c1ccc(-c2ccc(-n3c4ccccc4c4ccccc43)cc2)cc1 ; c1cc(-c2ccc(-n3c4ccccc4c4ccccc43)cc2)cc(-c2cccc(-c3ccc(-n4c5ccccc5c5ccccc54)cc3)c2)c1 ; c1ccc(-c2cc(-c3ccc(-n4c5ccccc5c5ccccc54)cc3)cc(-c3cc(-c4ccccc4)cc(-c4ccc(-n5c6ccccc6c6ccccc65)cc4)c3)c2)cc1 ; c1ccc(-c2cc(-c3ccc(-n4c5ccccc5c5ccccc54)cc3)cc(-c3cccc(-c4ccc(-n5c6ccccc6c6ccccc65)cc4)c3)c2)cc1]

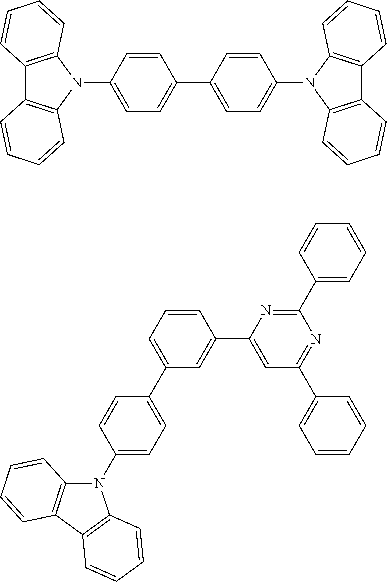 [SMILES: c1ccc2c(c1)c1ccccc1n2-c1ccc(-c2ccc(-n3c4ccccc4c4ccccc43)cc2)cc1 ; c1ccc(-c2cc(-c3cccc(-c4ccc(-n5c6ccccc6c6ccccc65)cc4)c3)nc(-c3ccccc3)n2)cc1]

### Electron-Donating Dopant

The organic EL device in an aspect of the invention preferably comprises an electron-donating dopant at an interfacial region between the cathode and the emitting unit. With such a construction, the organic EL device has an improved luminance and an elongated lifetime. The electron-donating dopant comprises a metal having a work function of 3.8 eV or less and examples thereof include at least one selected from an alkali metal, an alkali metal complex, an alkali metal compound, an alkaline earth metal, an alkaline earth metal complex, an alkaline earth metal compound, a rare earth metal, a rare earth metal complex, and a rare earth metal compound.

Examples of the alkali metal include Na (work function: 2.36 eV), K (work function: 2.28 eV), Rb (work function: 2.16 eV), and Cs (work function: 1.95 eV), with those having a work function of 2.9 eV or less being particularly preferred. Of the above, preferred are K, Rb, and Cs, more preferred are Rb and Cs, and most preferred is Cs. Examples of the alkaline earth metal include Ca (work function: 2.9 eV), Sr (work function: 2.0 to 2.5 eV), and Ba (work function: 2.52 eV), with those having a work function of 2.9 eV or less being particularly preferred. Examples of the rare earth metal include Sc, Y, Ce, Tb, and Yb, with those having a work function of 2.9 eV or less being particularly preferred.

Examples of the alkali metal compound include an alkali oxide, such as Li₂O, Cs₂O, and K₂O, and an alkali halide, such as LiF, NaF, CsF, and KF, with LiF, Li₂O, and NaF being preferred. Examples of the alkaline earth metal compound include BaO, SrO, CaO, and a mixture thereof, such as BaₓSr₁₋ₓO (0<x<1) and BaₓCa₁₋ₓO (0<x<1), with BaO, SrO, and CaO being preferred. Examples of the rare earth metal compound include YbF₃, ScF₃, ScO3, Y₂O₃, Ce₂O₃, GdF₃, and TbF₃, with YbF₃, ScF₃, and TbF₃ being preferred.

Examples of the alkali metal complex, the alkaline earth metal complex, and the rare earth metal are not particularly limited as long as containing at least one metal ion selected from an alkali metal ion, an alkaline earth metal ion, and a rare earth metal ion, respectively. The ligand is preferably, but not limited to, quinolinol, benzoquinolinol, acridinol, phenanthridinol, hydroxyphenyloxazole, hydroxyphenylthiazole, hydroxydiaryloxadiazole, hydroxydiarylthiadiazole, hydroxyphenylpyridine, hydroxyphenylbenzimidazole, hydroxybenzotriazole, hydroxyfulborane, bipyridyl, phenanthroline, phthalocyanine, porphyrin, cyclopentadiene, β-diketones, azomethines, and derivatives thereof.

The electron-donating dopant is added to the interfacial region preferably into a form of layer or island preferably by co-depositing the electron-donating dopant together with an organic compound for forming the interfacial region (a light emitting material and an electron injecting material) by a resistance heating deposition method, thereby dispersing the electron-donating dopant into the organic compound. The disperse concentration expressed by the molar ratio of organic compound:electron-donating dopant is 100:1 to 1:100 and preferably 5:1 to 1:5.

When the electron-donating dopant is formed into a form of layer, an organic compound (a light emitting material or an electron injecting material) is made into a layer to form an interfacial organic layer, and then, the electron-donating dopant alone is deposited by a resistance heating deposition method into a layer having a thickness of preferably 0.1 to 15 nm. When the electron-donating dopant is formed into a form of island, an organic compound (a light emitting material or an electron injecting material) is made into a form of island to form an interfacial organic layer, and then, the electron-donating dopant alone is deposited by a resistance heating deposition method into a form of island having a thickness of preferably 0.05 to 1 nm.

The molar ratio of the organic compound and the electron-donating dopant in the organic EL device in an aspect of the invention is preferably 5:1 to 1:5 and more preferably 2:1 to 1:2.

### Electron Transporting Layer

The electron transporting layer is an organic layer disposed between a light emitting layer and a cathode and transports electrons from the cathode to the light emitting layer. If two or more electron transporting layers are provided, the organic layer closer to the cathode may be defined as an electron injecting. The electron injecting layer injects electrons from the cathode to the organic layer unit efficiently. The compound (1) may be used in the electron transporting layer (second charge transporting layer) as an electron transporting material.

An aromatic heterocyclic compound having one or more heteroatoms in its molecule is preferably used as an electron transporting material for use in the electron transporting layer, and a nitrogen-containing ring derivative is particularly preferred. The nitrogen-containing ring derivative is preferably an aromatic ring compound having a nitrogen-containing 6- or 5-membered ring, or a fused aromatic ring compound having a nitrogen-containing 6- or 5-membered ring.

The nitrogen-containing ring derivative is preferably a metal chelate complex having a nitrogen-containing ring represented by formula (A):

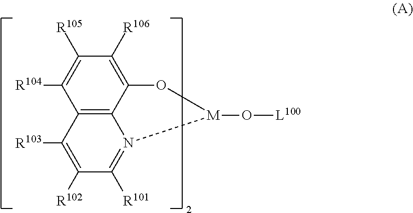

wherein R¹⁰¹ to R¹⁰⁵ each independently represent a hydrogen atom, a halogen atom, a hydroxyl group, an amino group, a hydrocarbon group having 1 to 40, preferably 1 to 20, more preferably 1 to 10, and still more preferably 1 to 5 carbon atoms, an alkoxy group having 1 to 40, preferably 1 to 20, more preferably 1 to 10, and still more preferably 1 to 5 carbon atoms, an aryloxy group having 6 to 50, preferably 6 to 20, and more preferably 6 to 12 ring carbon atoms, an alkoxycarbonyl group having 2 to 40, preferably 2 to 20, more preferably 2 to 10, and still more preferably 2 to 5 carbon atoms, or an aromatic heterocyclic group having 5 to 50, preferably 5 to 30, and more preferably 5 to 20 ring atoms, each optionally having a substituent.

The halogen atom may include fluorine, chlorine, bromine, and iodine.

The substituted amino group may include an alkylamino group, an arylamino group, and an aralkylamino group.

The alkylamino group and the aralkylamino group are represented by —NQ¹Q². Q¹ and Q² each independently represent an alkyl group having 1 to 20 carbon atoms or an aralkyl group having 1 to 20 carbon atoms. One of Q¹ and Q² may be a hydrogen atom.

The arylamino group is represented by —NAr^{1′}Ar^{2′}, wherein Ar^{1′} and Ar^{2′} each independently represent a non-fused aromatic hydrocarbon group or a fused aromatic hydrocarbon group, each having 6 to 50 carbon atoms. One of Ar^{1′} and Ar^{2′} may be a hydrogen atom.

Examples of the hydrocarbon group having 1 to 40 carbon atoms include an alkyl group, an alkenyl group, a cycloalkyl group, an aryl group, and an aralkyl group.

The alkoxycarbonyl group is represented by —COOY′, wherein Y′ is an alkyl group having 1 to 20 carbon atoms.

M is aluminum (Al), gallium (Ga), or indium (In), with In being preferred.

L¹⁰⁰ is a group represented by formula (A′) or (A″):

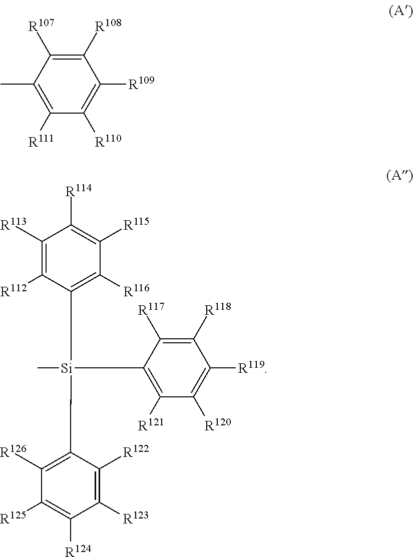

R¹⁰⁷ to R¹¹¹ of formula (A′) each independently represent a hydrogen atom or a substituted or unsubstituted hydrocarbon group having 1 to 40, preferably 1 to 20, more preferably 1 to 10, and still more preferably 1 to 5 carbon atoms, wherein two or more selected from R¹⁰⁷ to R¹¹¹ may be bonded to each other form a ring structure. R¹¹² to R¹²⁶ of formula (A″) each independently represent a hydrogen atom or a substituted or unsubstituted hydrocarbon group having 1 to 40, preferably 1 to 20, more preferably 1 to 10, and still more preferably 1 to 5 carbon atoms, wherein two or more selected from R¹¹² to R¹²⁶ may be bonded to each other form a ring structure.

Examples of the hydrocarbon group having 1 to 40 carbon atoms for R¹⁰⁷ to R¹²⁶ of formulae (A′) and (A″) are the same as those described above with respect to R¹⁰¹ to R¹⁰⁶ of formula (A). Examples of the divalent group formed by two or more selected from R¹⁰⁷ to R¹¹¹, or two or more selected from R¹¹² to R¹²⁶ which completes a ring structure include a tetramethylene group, a pentamethylene group, a hexamethylene group, a diphenylmethane-2,2′-diyl group, a diphenylethane-3,3′-diyl group, and a diphenylpropane-4,4′-diyl group.

The electron transporting compound for use in the electron transporting layer is preferably a metal complex including 8-hydroxyquinoline or its derivative, an oxadiazole derivative, or a nitrogen-containing heterocyclic derivative. Examples of the metal complex including 8-hydroxyquinoline or its derivative include a metal chelate oxinoid including a chelated oxine (generally, 8-quinolinol or 8-hydroxyquinoline), for example, tris(8-quinolinol)aluminum. Examples of the oxadiazole derivative are shown below:

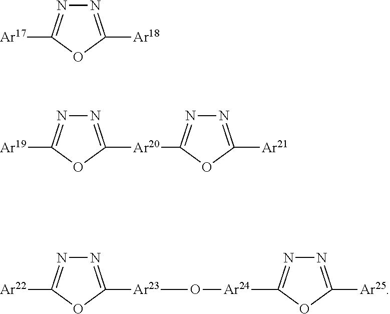

wherein Ar¹⁷, Ar¹⁸, Ar¹⁹, Ar²¹, Ar²², and Ar²⁵ are each a substituted or unsubstituted aromatic hydrocarbon group or a substituted or unsubstituted fused aromatic hydrocarbon group each having 6 to 50 carbon atoms, and Ar¹⁷ and Ar¹⁸, Ar¹⁹ and Ar²¹, and Ar²² and Ar²⁵ may be the same or different, respectively. Examples of the aromatic hydrocarbon group and the fused aromatic hydrocarbon group include a phenyl group, a naphthyl group, a biphenyl group, an anthranyl group, a perylenyl group, and a pyrenyl group. The optional substituent may be an alkyl group having 1 to 10 carbon atoms, an alkoxyl group having 1 to 10 carbon atoms or a cyano group.

Ar²⁰, Ar²³, and Ar²⁴ are each a substituted or unsubstituted divalent aromatic hydrocarbon group or a substituted or unsubstituted divalent fused aromatic hydrocarbon group each having 6 to 50 carbon atoms, and Ar²³ and Ar²⁴ may be the same or different. Examples of the divalent aromatic hydrocarbon group or the divalent fused aromatic hydrocarbon group include a phenylene group, a naphthylene group, a biphenylene group, an anthranylene group, a perylenylene group, and a pyrenylene group. The optional substituent may be an alkyl group having 1 to 10 carbon atoms, an alkoxyl group having 1 to 10 carbon atoms or a cyano group.

An electron transporting compound which has a good thin film-forming property is preferably used. Examples thereof are shown below:

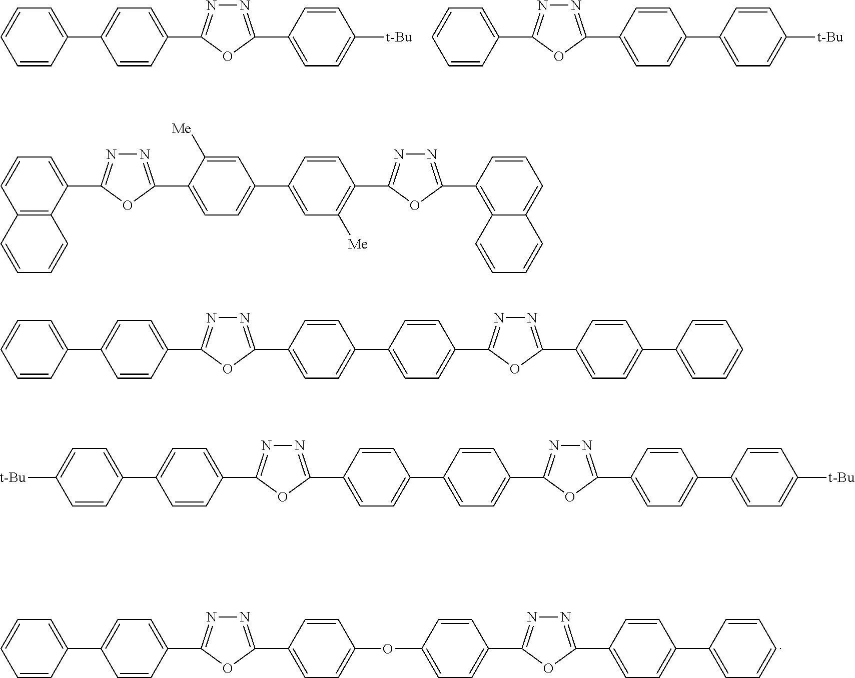

Examples of the nitrogen-containing heterocyclic derivative for use as the electron transporting compound include a nitrogen-containing heterocyclic derivative having the following formulae but exclusive of a metal complex, for example, a compound having a 5- or 6-membered ring which includes a skeleton represented by formula (B) or having a structure represented by formula (C):

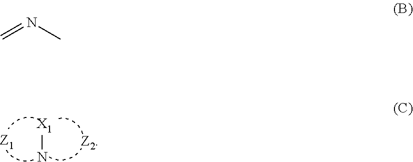

wherein X₁ is a carbon atom or a nitrogen atom and Z₁ and Z₂ each independently represent a group of atoms for completing the nitrogen-containing heterocyclic ring.

The nitrogen-containing heterocyclic derivative is more preferably an organic compound which has a nitrogen-containing aromatic polycyclic ring comprising a 5-membered ring or a 6-membered ring. If two or more nitrogen atoms are included, the nitrogen-containing aromatic polycyclic compound preferably has a skeleton of a combination of formulae (B) and (C) or a combination of formulae (B) and (D):

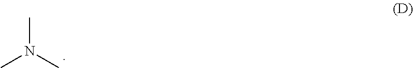

The nitrogen-containing group of the nitrogen-containing aromatic polycyclic compound is selected, for example, from the nitrogen-containing heterocyclic groups shown below:

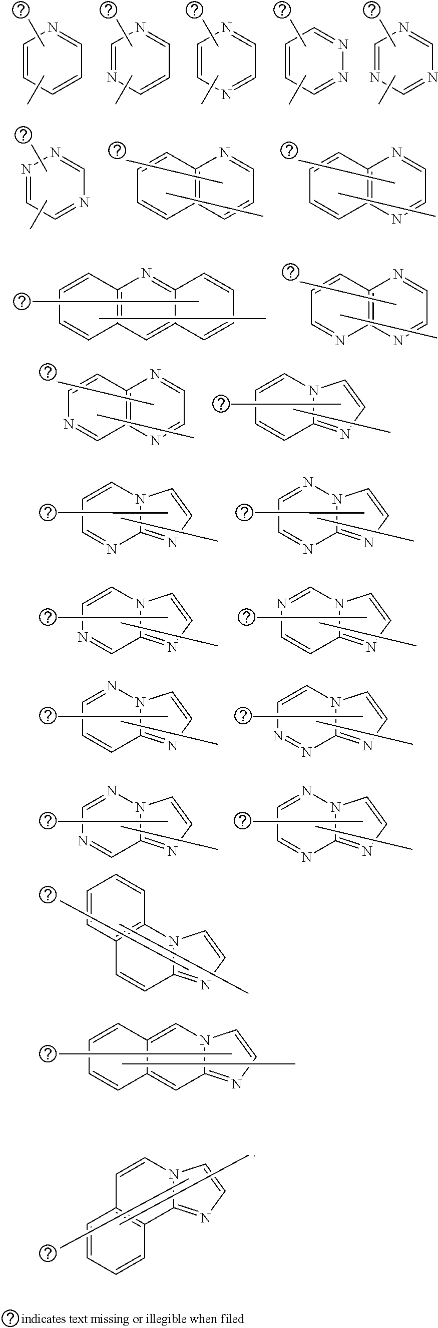

wherein R′″ is an aromatic hydrocarbon group having 6 to 40, preferably 6 to 30, more preferably 6 to 20, and still more preferably 6 to 12 ring carbon atoms, a fused aromatic hydrocarbon group having 6 to 40, preferably 6 to 30, more preferably 6 to 20, and still more preferably 6 to 12 ring carbon atoms, an aromatic heterocyclic group having 5 to 40, preferably 5 to 30, more preferably 5 to 20, and still more preferably 5 to 12 ring atoms, a fused aromatic heterocyclic group having 5 to 40, preferably 5 to 30, more preferably 5 to 20, and still more preferably 5 to 12 ring atoms, an alkyl group having 1 to 20, preferably 1 to 10, and more preferably 1 to 5 carbon atoms, or an alkoxy group having 1 to 20, preferably 1 to 10, and more preferably 1 to 5 carbon atoms; and

n₁ is an integer of 0 to 5 and when n₁ is an integer of 2 or more, groups R′″ may be the same or different.

A nitrogen-containing heterocyclic derivative represented by formula (D1) is also preferred:

HAr-L¹⁰¹-Ar¹⁰¹—Ar¹⁰² (D1)

wherein:

HAr is a substituted or unsubstituted nitrogen-containing heterocyclic group having 5 to 40, preferably 5 to 30, more preferably 5 to 20, and still more preferably 5 to 12 ring atoms;

L¹⁰¹ is a single bond, a substituted or unsubstituted aromatic hydrocarbon group or fused aromatic hydrocarbon group each having 6 to 40, preferably 6 to 30, more preferably 6 to 20, and still more preferably 6 to 12 ring carbon atoms, a substituted or unsubstituted aromatic heterocyclic group having 5 to 40, preferably 5 to 30, more preferably 5 to 20, and still more preferably 5 to 12 ring atoms, or a substituted or unsubstituted fused aromatic heterocyclic group having 6 to 40, preferably 6 to 30, more preferably 6 to 20, and still more preferably 6 to 12 ring carbon atoms; and

Ar¹⁰¹ is a substituted or unsubstituted divalent aromatic hydrocarbon group having 6 to 40, preferably 6 to 30, more preferably 6 to 20, and still more preferably 6 to 12 ring carbon atoms; and

Ar¹⁰² is a substituted or unsubstituted aromatic hydrocarbon group having 6 to 40, preferably 6 to 30, more preferably 6 to 20, and still more preferably 6 to 14 ring carbon atoms, a substituted or unsubstituted fused aromatic hydrocarbon group having 6 to 40, preferably 6 to 30, more preferably 6 to 20, and still more preferably 6 to 12 ring carbon atoms, a substituted or unsubstituted aromatic heterocyclic group having 5 to 40, preferably 5 to 30, more preferably 5 to 20, and still more preferably 5 to 12 ring atoms, or a substituted or unsubstituted fused aromatic heterocyclic group having 5 to 40, preferably 5 to 30, more preferably 5 to 20, and still more preferably 5 to 12 ring atoms.

HAr is selected, for example, from the following groups:

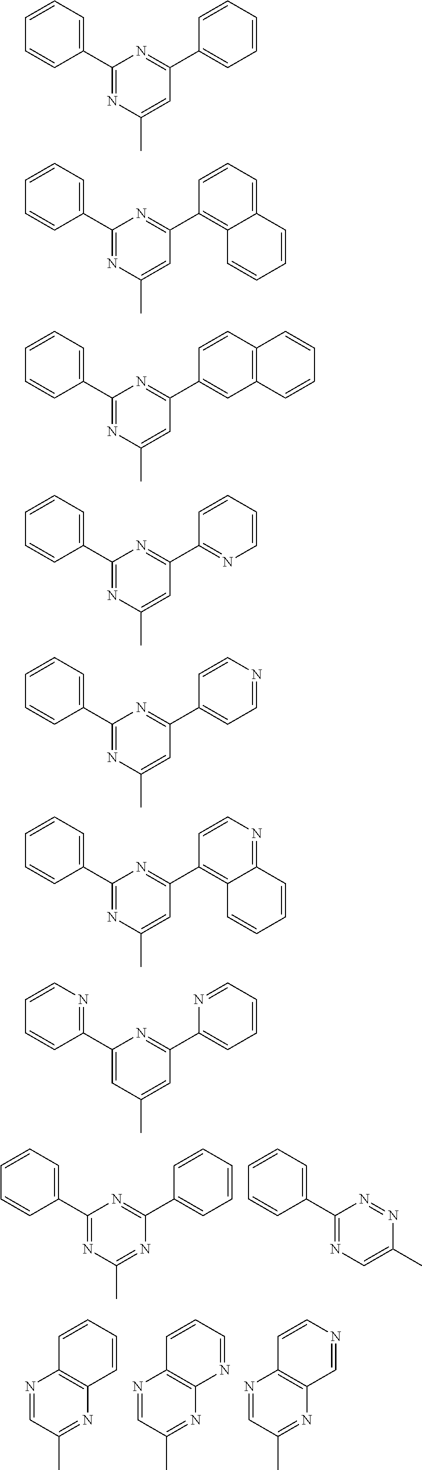

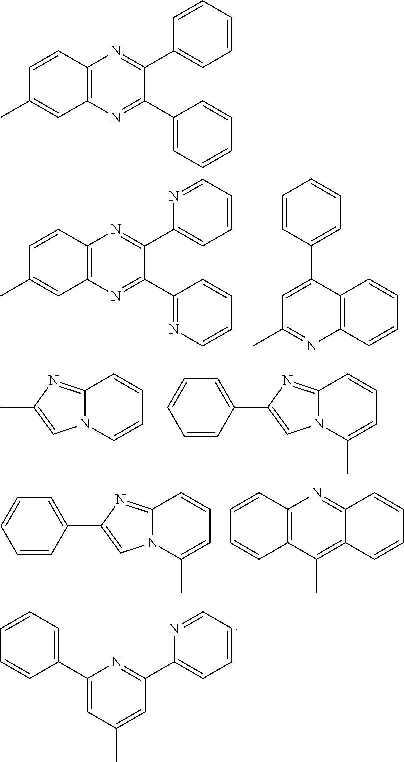

L¹⁰¹ is selected, for example, from the following groups:

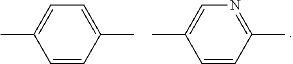

Ar¹⁰¹ is selected, for example, from the group represented by formula (D2) or (D3):

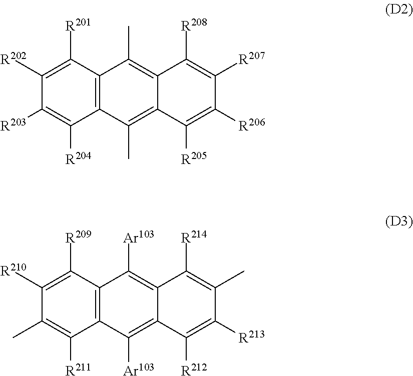

wherein:

R²⁰¹ to R²¹⁴ are each independently a hydrogen atom, a halogen atom, a substituted or unsubstituted alkyl group having 1 to 20, preferably 1 to 10, and more preferably 1 to 5 carbon atoms, a substituted or unsubstituted alkoxy group having 1 to 20, preferably 1 to 10, and more preferably 1 to 5 carbon atoms, a substituted or unsubstituted aryloxy group having 6 to 40, preferably 6 to 30, more preferably 6 to 20, and still more preferably 6 to 12 ring carbon atoms, a substituted or unsubstituted aromatic hydrocarbon group having 6 to 40, preferably 6 to 30, more preferably 6 to 20, and still more preferably 6 to 12 ring carbon atoms, a substituted or unsubstituted fused aromatic hydrocarbon group having 6 to 40, preferably 6 to 30, more preferably 6 to 20, and still more preferably 6 to 12 ring carbon atoms, a substituted or unsubstituted aromatic heterocyclic group having 5 to 40, preferably 5 to 30, more preferably 5 to 20, and still more preferably 5 to 12 ring atoms, or a substituted or unsubstituted fused aromatic heterocyclic group having 5 to 40, preferably 5 to 30, more preferably 5 to 20, and still more preferably 5 to 12 ring atoms; and

Ar¹⁰³ is a substituted or unsubstituted aromatic hydrocarbon group having 6 to 40, preferably 6 to 30, more preferably 6 to 20, and still more preferably 6 to 12 ring carbon atoms, a substituted or unsubstituted fused aromatic hydrocarbon group having 6 to 40, preferably 6 to 30, more preferably 6 to 20, and still more preferably 6 to 12 ring carbon atoms, a substituted or unsubstituted aromatic heterocyclic group having 5 to 40, preferably 5 to 30, more preferably 5 to 20, and still more preferably 5 to 12 ring atoms, or a substituted or unsubstituted fused aromatic heterocyclic group having 5 to 40, preferably 5 to 30, more preferably 5 to 20, and still more preferably 5 to 12 ring atoms.

Ar¹⁰² is selected, for example, from the following groups:

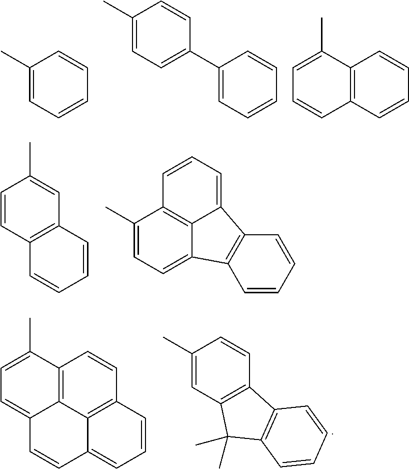

In addition, the following compound is preferably used as the nitrogen-containing aromatic polycyclic compound for use as the electron transporting compound:

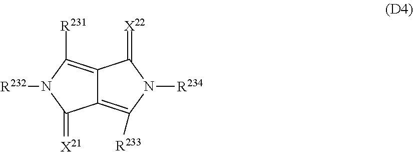

wherein R²³¹ to R²³⁴ each independently represent a hydrogen atom, a substituted or unsubstituted aliphatic group having 1 to 20 carbon atoms, a substituted or unsubstituted alicyclic group having 3 to 20 carbon atoms, a substituted or unsubstituted aromatic ring group having 6 to 50 carbon atoms, or a substituted or unsubstituted heterocyclic group having 3 to 50 carbon atoms; and X²¹ and X²² each independently represent an oxygen atom, a sulfur atom, or a dicyanomethylene group.

Further, the following compound is also suitable as the electron transporting compound:

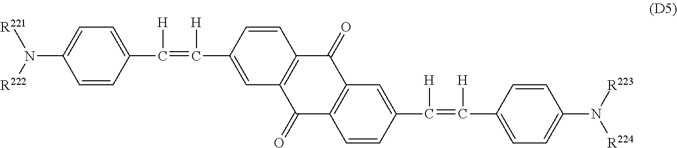

wherein R²²¹, R²²², R²²³, and R²²⁴ may be the same or different and each represent an aromatic hydrocarbon group or a fused aromatic hydrocarbon group each represented by formula (D6):

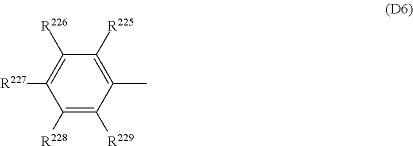

wherein R²²⁵, R²²⁶, R²²⁷, R²²⁸, and R²²⁹ may be the same or different and each represent a hydrogen atom, a saturated or unsaturated alkoxyl group having 1 to 20 carbon atoms, a saturated or unsaturated alkyl group having 1 to 20 carbon atoms, an amino group, or an alkylamino group having 1 to 20 carbon atoms; and at least one selected from R²²⁵, R²²⁶, R²²⁷, R²²⁸, and R²²⁹ represents a group other than a hydrogen atom.

Further, a polymer including the nitrogen-containing heterocychlic group or the nitrogen-containing heterocyclic derivative is also usable as the electron transporting compound.

The electron transporting layer of the organic EL device in an aspect of the invention preferably comprises at least one compound selected from the nitrogen-containing heterocyclic derivatives represented by formulae (E) to (G):

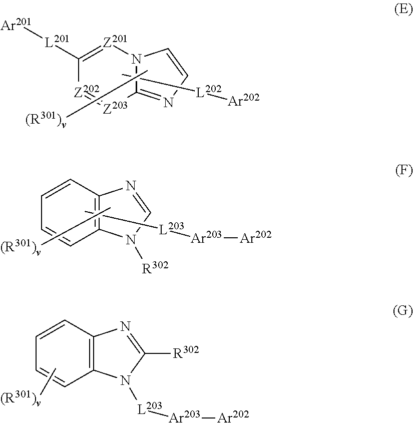

wherein Z²⁰¹, Z²⁰² and Z²⁰³ each independently represent a nitrogen atom or a carbon atom;

R³⁰¹ and R³⁰² each independently represent a substituted or unsubstituted aryl group having 6 to 50, preferably 6 to 30, more preferably 6 to 20, and still more preferably 6 to 12 ring carbon atoms, a substituted or unsubstituted heteroaryl group having 5 to 50, preferably 5 to 30, more preferably 5 to 20, and still more preferably 5 to 12 ring atoms, a substituted or unsubstituted alkyl group having 1 to 20 carbon atoms, a substituted or unsubstituted haloalkyl group having 1 to 20 carbon atoms, or a substituted or unsubstituted alkoxy group having 1 to 20 carbon atoms;

v is an integer of 0 to 5, when v is an integer of 2 or more, groups R³⁰¹ may be the same or different, and adjacent two groups R³⁰¹ may bond to each other to form a substituted or unsubstituted hydrocarbon ring;

Ar²⁰¹ represents a substituted or unsubstituted aryl group having 6 to 50, preferably 6 to 30, more preferably 6 to 20, and still more preferably 6 to 12 ring carbon atoms or a substituted or unsubstituted heteroaryl group having 5 to 50, preferably 5 to 30, more preferably 5 to 20, and still more preferably 5 to 12 ring atoms;

Ar²⁰² represents a hydrogen atom, a substituted or unsubstituted alkyl group having 1 to 20, preferably 1 to 10, and more preferably 1 to 5 carbon atoms, a substituted or unsubstituted haloalkyl group having 1 to 20, preferably 1 to 10, and more preferably 1 to 5 carbon atoms, a substituted or unsubstituted alkoxy group having 1 to 20 carbon atoms, a substituted or unsubstituted aryl group having 6 to 50, preferably 6 to 30, more preferably 6 to 20, and still more preferably 6 to 12 ring carbon atoms, or a substituted or unsubstituted heteroaryl group having 5 to 50, preferably 5 to 30, more preferably 5 to 20, and still more preferably 5 to 12 ring atoms;

provided that one of Ar²⁰¹ and Ar²⁰² is a substituted or unsubstituted fused aromatic hydrocarbon ring group having 10 to 50, preferably 10 to 30, and more preferably 10 to 20 ring carbon atoms or a substituted or unsubstituted fused aromatic heterocyclic group having 9 to 50, preferably 9 to 30, and more preferably 9 to 20 ring atoms;

Ar²⁰³ represents a substituted or unsubstituted arylene group having 6 to 50, preferably 6 to 30, more preferably 6 to 20, and still more preferably 6 to 12 ring carbon atoms or a substituted or unsubstituted heteroarylene group having 5 to 50, preferably 5 to 30, more preferably 5 to 20, and still more preferably 5 to 12 ring atoms; and

L²⁰¹, L²⁰², and L²⁰³ each independently represent a single bond, a substituted or unsubstituted arylene group having 6 to 50, preferably 6 to 30, more preferably 6 to 20, and still more preferably 6 to 12 ring carbon atoms or a substituted or unsubstituted divalent fused aromatic heterocyclic group having 9 to 50, pre 9 to 30, and more preferably 9 to 20 ring atoms.

Examples of the aryl group having 6 to 50 ring carbon atoms include a phenyl group, a naphthyl group, an anthryl group, a phenanthryl group, a naphthacenyl group, a chrysenyl group, pyrenyl group, a biphenyl group, a terphenyl group, a tolyl group, a fluoranthenyl group, and a fluorenyl group.

Examples of the heteroaryl group having 5 to 50 ring atoms include a pyrrolyl group, a furyl group, a thiophenyl group, a silolyl group, a pyridyl group, a quinolyl group, an isoquinolyl group, a benzofuryl group, an imidazolyl group, a pyrimidyl group, a carbazolyl group, a selenophenyl group, an oxadiazolyl group, a triazolyl group, a pyrazinyl group, a pyridazinyl group, a triazinyl group, a quinoxalinyl group, an acridinyl group, an imidazo[1,2-a]pyridinyl group, and an imidazo[1,2-a]pyrimidinyl.

Examples of the alkyl group having 1 to 20 carbon atoms include a methyl group, an ethyl group, a propyl group, a butyl group, a pentyl group, and a hexyl group.

Examples of the haloalkyl group having 1 to 20 carbon atoms include the groups obtained by replacing one or more hydrogen atoms of the alkyl group mentioned above with at least one halogen atom selected from fluorine, chlorine, iodine, and bromine.

Examples of the alkyl moiety of the alkoxy group having 1 to 20 carbon atoms include the alkyl group mentioned above.

Examples of the arylene groups include the groups obtained by removing one hydrogen atom from the aryl group mentioned above.

Examples of the divalent fuse aromatic heterocyclic group having 9 to 50 ring atoms include the groups obtained by removing one hydrogen atom from the fused aromatic heterocycic group mentioned above with respect to the heteroaryl group.

The thickness of the electron transporting layer is preferably 1 to 100 nm, but not particularly limited thereto.

The electron injecting layer optionally formed adjacent to the electron transporting layer preferably comprises an inorganic compound, such as an insulating material and a semiconductor, in addition to the nitrogen-containing ring derivative. The electron injecting layer comprising the insulating material or the semiconductor effectively prevents the leak of electric current to enhance the electron injecting properties.

The insulating material is preferably at least one metal compound selected from the group consisting of an alkali metal chalcogenide, an alkaline earth metal chalcogenide, an alkali metal halide and an alkaline earth metal halide. The electron injecting properties of the electron injecting layer are further enhanced when the alkali metal chalcogenide, etc. is used in the electron injecting layer. Examples of preferred alkali metal chalcogenide include Li₂O, K₂O, Na₂S, Na₂Se and Na₂OO, and examples of preferred alkaline earth metal chalcogenide include CaO, BaO, SrO, BeO, BaS and CaSe. Examples of preferred alkali metal halide include LiF, NaF, KF, LiCl, KCl and NaCl. Examples of preferred alkaline earth metal halide include fluorides, such as CaF₂, BaF₂, SrF₂, MgF₂ and BeF₂, and halides other than fluorides.

Examples of the semiconductor include an oxide, a nitride and an oxynitride of at least one element selected from the group consisting of Ba, Ca, Sr, Yb, Al, Ga, In, Li, Na, Cd, Mg, Si, Ta, Sb and Zn. The semiconductor may be used alone or in combination of two or more. The inorganic compound included in the electron injecting layer preferably forms a microcrystalline or amorphous insulating thin film. The electron injecting layer formed from such an insulating thin film decreases the pixel defects, such as dark spots, because the insulating thin film is highly uniform. Examples of such an inorganic compound include the alkali metal chalcogenide, the alkaline earth metal chalcogenide, the alkali metal halide and the alkaline earth metal halide.

The thickness of a layer comprising the insulating material or the semiconductor is preferably about 0.1 to 15 nm. In an embodiment of the invention, the electron injecting layer may comprise the electron-donating dopant mentioned above.

### Hole Transporting Layer

The hole transporting layer is an organic layer formed between a light emitting layer and an anode and has a function of transporting holes from the anode to the light emitting layer. If two or more hole transporting layers are provided, the organic layer closer to the anode may be defined as the hole injecting layer in some cases. The hole injecting layer has a function of efficiently injecting holes from the anode to the organic layer unit. In an embodiment of the invention, the compound (1) may be included in the hole transporting layer (first charge transporting layer) as a hole transporting material.

A preferred material for use in the hole transporting layer may include an aromatic amine compound, for example, an aromatic amine derivative represented by formula (H):

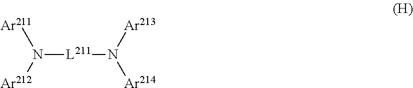

wherein:

Ar²¹¹ to Ar²¹⁴ each represent a substituted or unsubstituted aromatic hydrocarbon group having 6 to 50, preferably 6 to 30, more preferably 6 to 20, and still more preferably 6 to 12 ring carbon atoms, a substituted or unsubstituted fused aromatic hydrocarbon group having 6 to 50, preferably 6 to 30, more preferably 6 to 20, and still more preferably 6 to 12 ring carbon atoms, a substituted or unsubstituted aromatic heterocyclic group having 5 to 50, preferably 5 to 30, more preferably 5 to 20, and still more preferably 5 to 12 ring atoms, a substituted or unsubstituted fused aromatic heterocyclic group having 5 to 50, preferably 5 to 30, more preferably 5 to 20, and still more preferably 5 to 12 ring atoms, or a group wherein the aromatic hydrocarbon group or fused aromatic hydrocarbon group is bonded to the aromatic heterocyclic group or fused aromatic heterocyclic group;

Ar²¹¹ and Ar²¹² or Ar²¹³ and Ar²¹⁴ may be bonded to each other to form a saturated or unsaturated ring structure; and

L²¹¹ represents a substituted or unsubstituted aromatic hydrocarbon group having 6 to 50, preferably 6 to 30, more preferably 6 to 20, and still more preferably 6 to 12 ring carbon atoms, a substituted or unsubstituted fused aromatic hydrocarbon group having 6 to 50, preferably 6 to 30, more preferably 6 to 20, and still more preferably 6 to 12 ring carbon atoms, a substituted or unsubstituted aromatic heterocyclic group having 5 to 50, preferably 5 to 30, more preferably 5 to 20, and still more preferably 5 to 12 ring atoms, or a substituted or unsubstituted fused aromatic heterocyclic group having 5 to 50, preferably 5 to 30, more preferably 5 to 20, and still more preferably 5 to 12 ring atoms.

Examples of the compound represented by formula (H) are shown below:

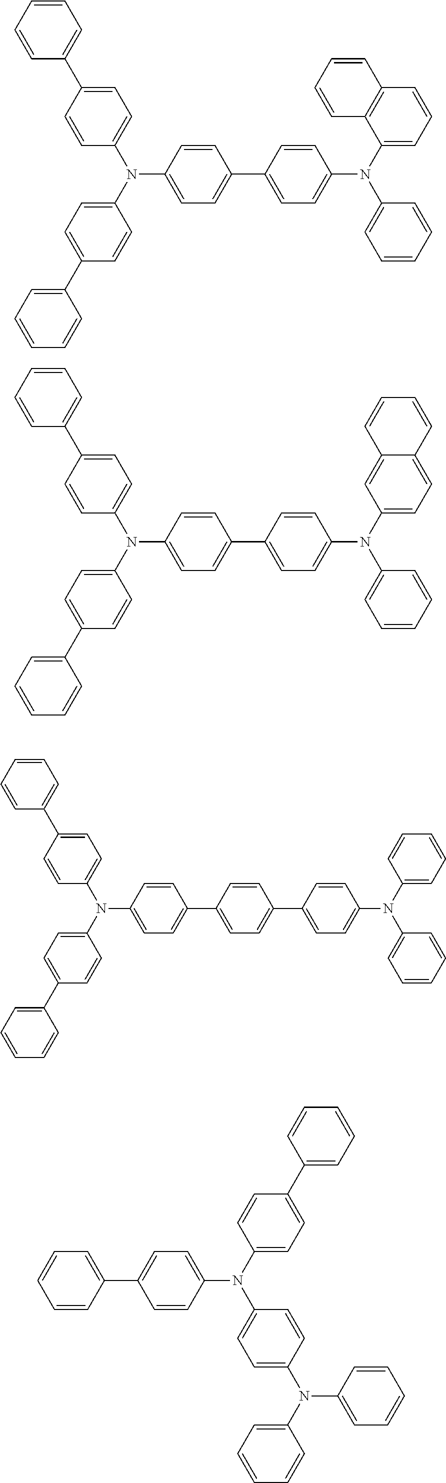

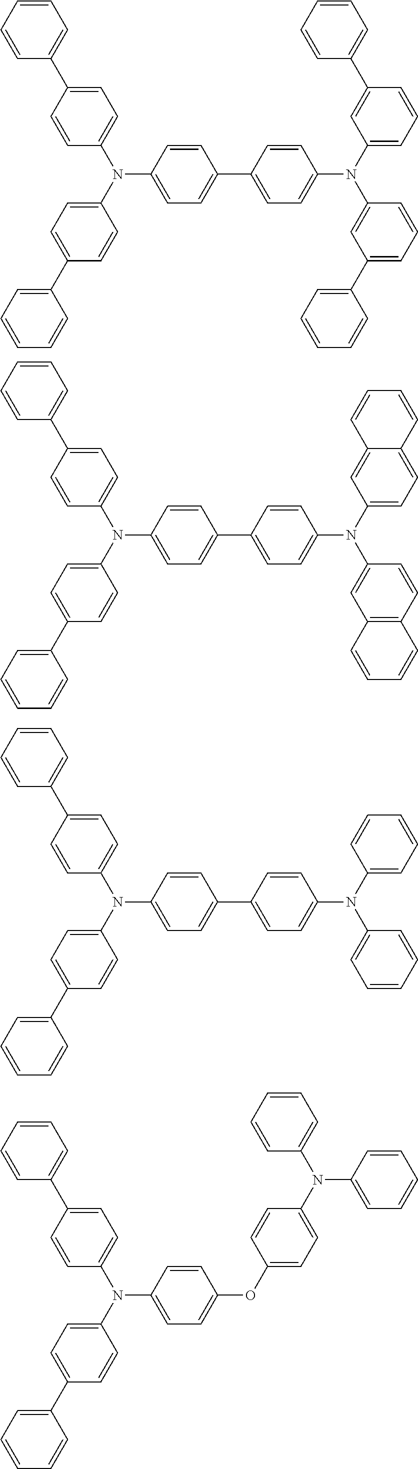

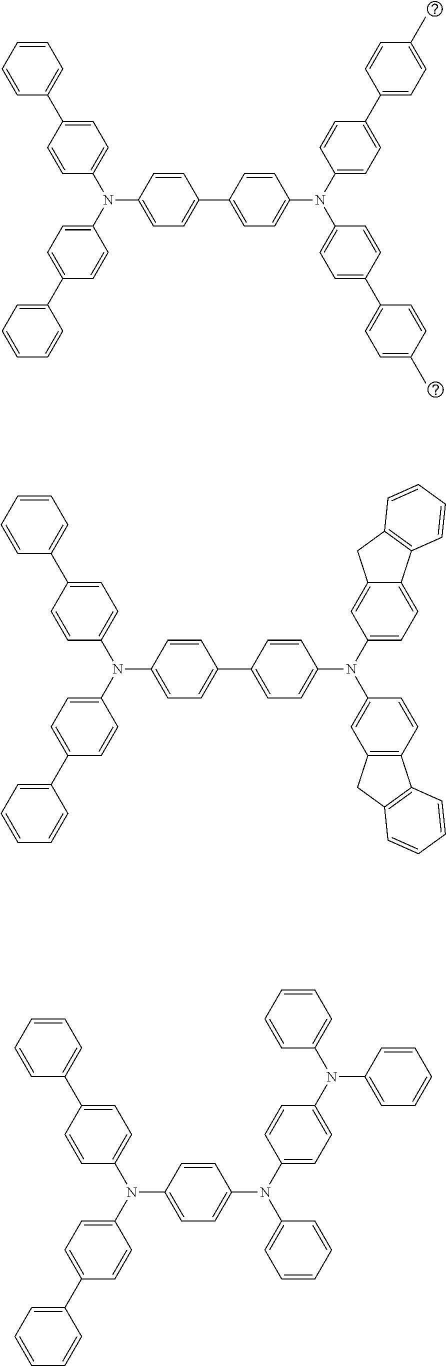 [SMILES: Cc1ccc(-c2ccc(N(c3ccc(-c4ccc(C)cc4)cc3)c3ccc(-c4ccc(N(c5ccc(-c6ccccc6)cc5)c5ccc(-c6ccccc6)cc5)cc4)cc3)cc2)cc1 ; c1ccc(-c2ccc(N(c3ccc(-c4ccccc4)cc3)c3ccc(-c4ccc(N(c5ccc6c(c5)Cc5ccccc5-6)c5ccc6c(c5)Cc5ccccc5-6)cc4)cc3)cc2)cc1 ; c1ccc(-c2ccc(N(c3ccc(-c4ccccc4)cc3)c3ccc(N(c4ccccc4)c4ccc(N(c5ccccc5)c5ccccc5)cc4)cc3)cc2)cc1]

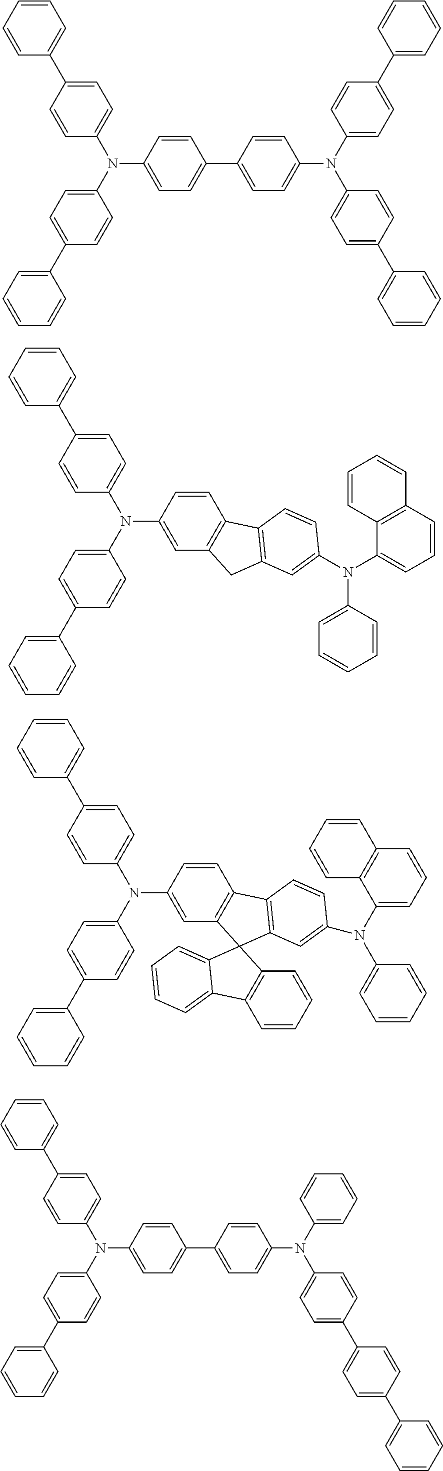

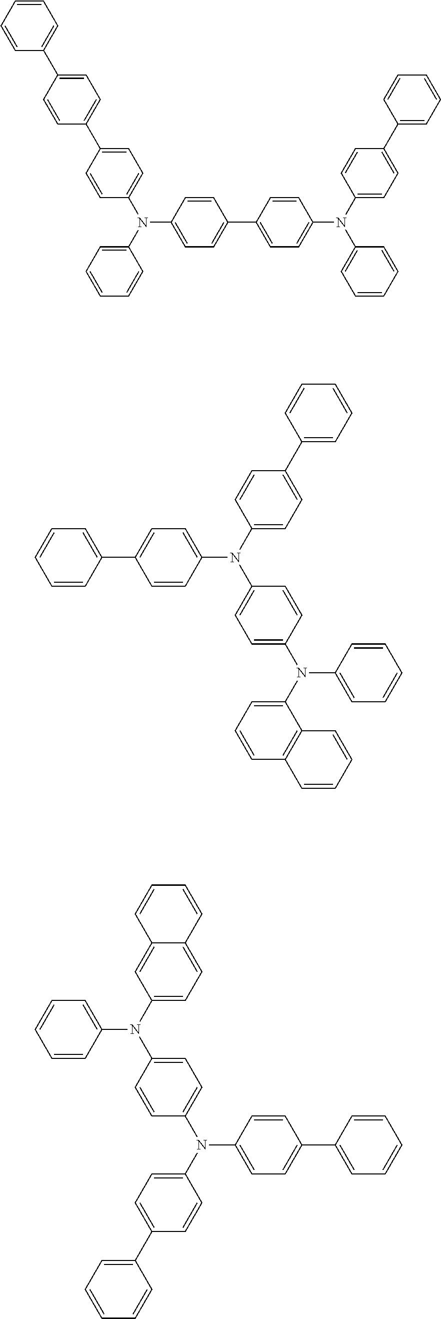 [SMILES: c1ccc(-c2ccc(-c3ccc(N(c4ccccc4)c4ccc(-c5ccc(N(c6ccccc6)c6ccc(-c7ccccc7)cc6)cc5)cc4)cc3)cc2)cc1 ; c1ccc(-c2ccc(N(c3ccc(-c4ccccc4)cc3)c3ccc(N(c4ccccc4)c4cccc5ccccc45)cc3)cc2)cc1 ; c1ccc(-c2ccc(N(c3ccc(-c4ccccc4)cc3)c3ccc(N(c4ccccc4)c4ccc5ccccc5c4)cc3)cc2)cc1]

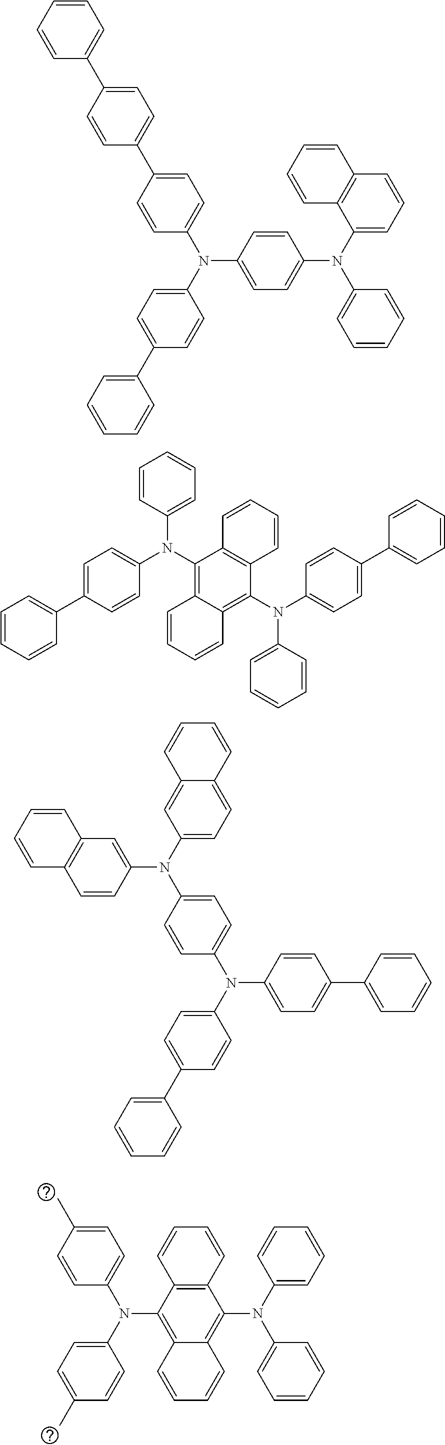

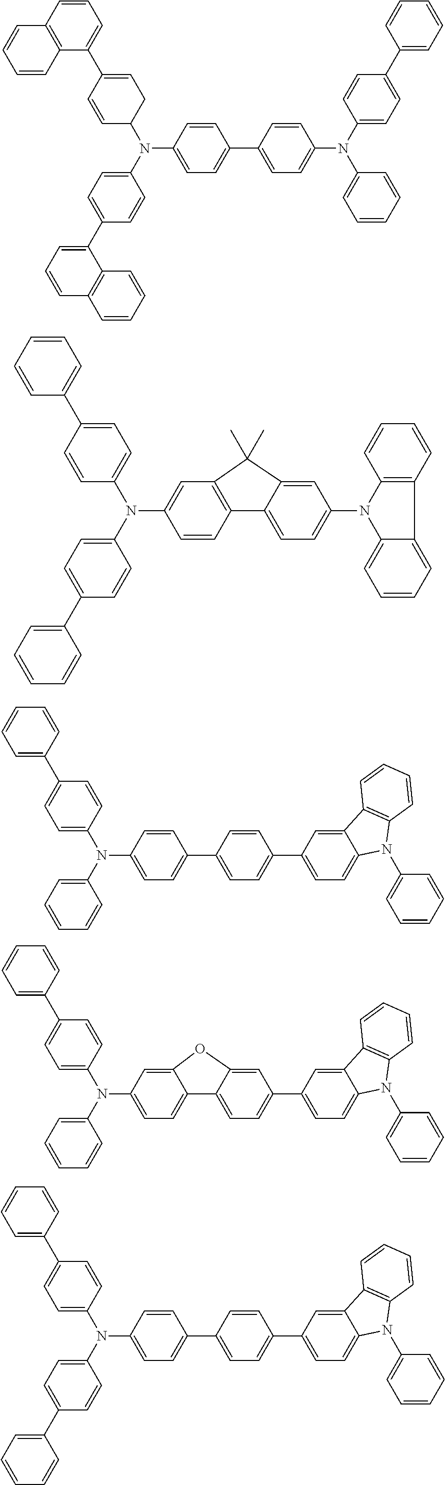

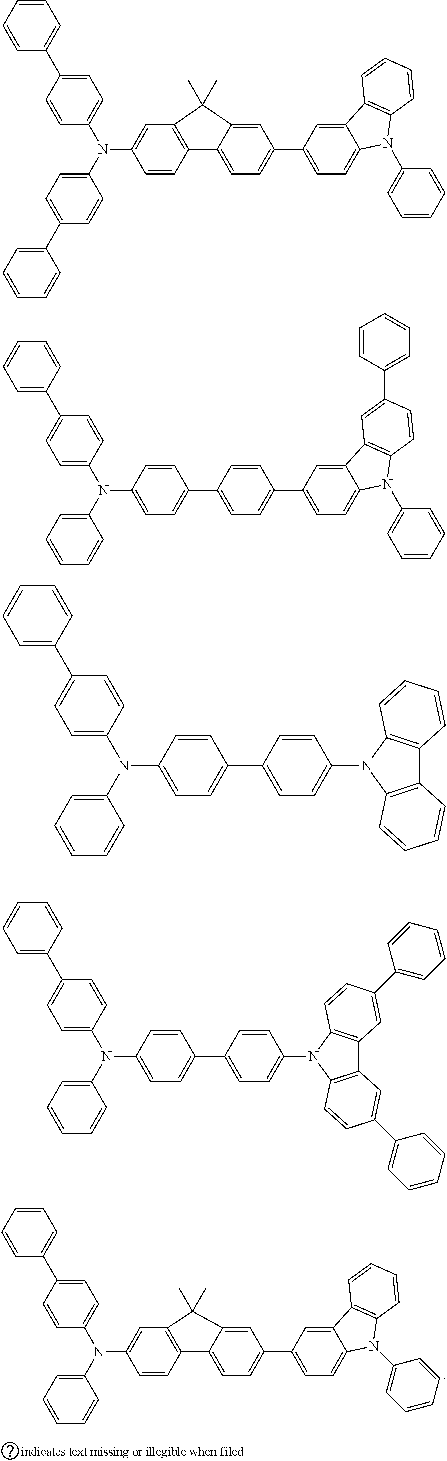

In addition, an aromatic amine represented by formula (J) is preferably used in the hole transporting layer:

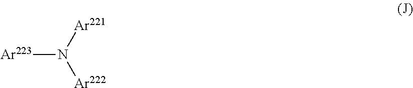

wherein Ar²²¹ to Ar²²³ are the same as defined above with respect to Ar²¹¹ to Ar²¹⁴ of formula (H). Examples of the compound represented by formula (J) are shown below, although not limited thereto.

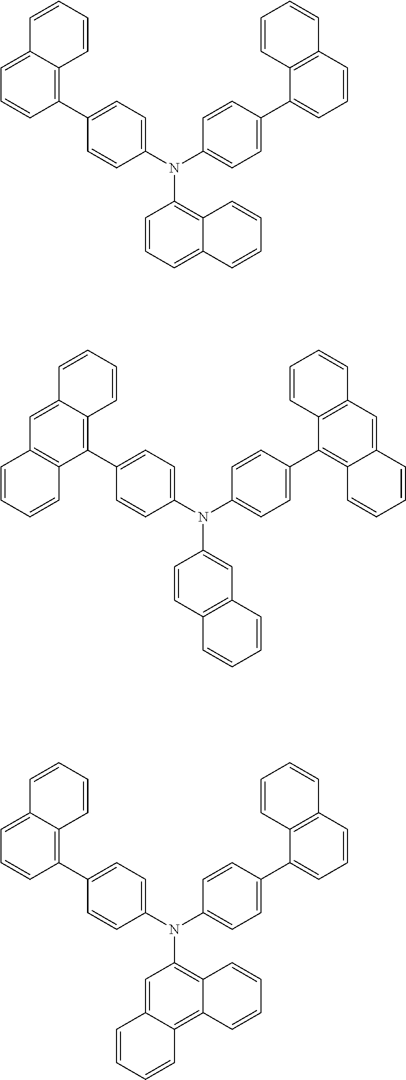 [SMILES: c1ccc2c(-c3ccc(N(c4ccc(-c5cccc6ccccc56)cc4)c4cccc5ccccc45)cc3)cccc2c1 ; c1ccc2cc(N(c3ccc(-c4c5ccccc5cc5ccccc45)cc3)c3ccc(-c4c5ccccc5cc5ccccc45)cc3)ccc2c1 ; c1ccc2c(-c3ccc(N(c4ccc(-c5cccc6ccccc56)cc4)c4cc5ccccc5c5ccccc45)cc3)cccc2c1]

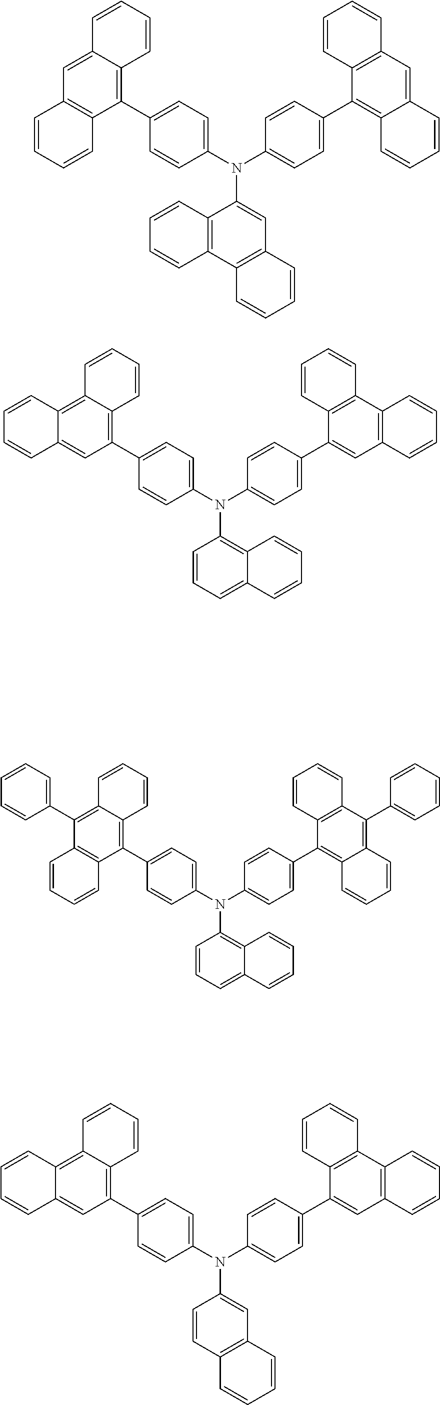 [SMILES: c1ccc2c(-c3ccc(N(c4ccc(-c5c6ccccc6cc6ccccc56)cc4)c4cc5ccccc5c5ccccc45)cc3)c3ccccc3cc2c1 ; c1ccc2c(N(c3ccc(-c4cc5ccccc5c5ccccc45)cc3)c3ccc(-c4cc5ccccc5c5ccccc45)cc3)cccc2c1 ; c1ccc(-c2c3ccccc3c(-c3ccc(N(c4ccc(-c5c6ccccc6c(-c6ccccc6)c6ccccc56)cc4)c4cccc5ccccc45)cc3)c3ccccc23)cc1 ; c1ccc2cc(N(c3ccc(-c4cc5ccccc5c5ccccc45)cc3)c3ccc(-c4cc5ccccc5c5ccccc45)cc3)ccc2c1]

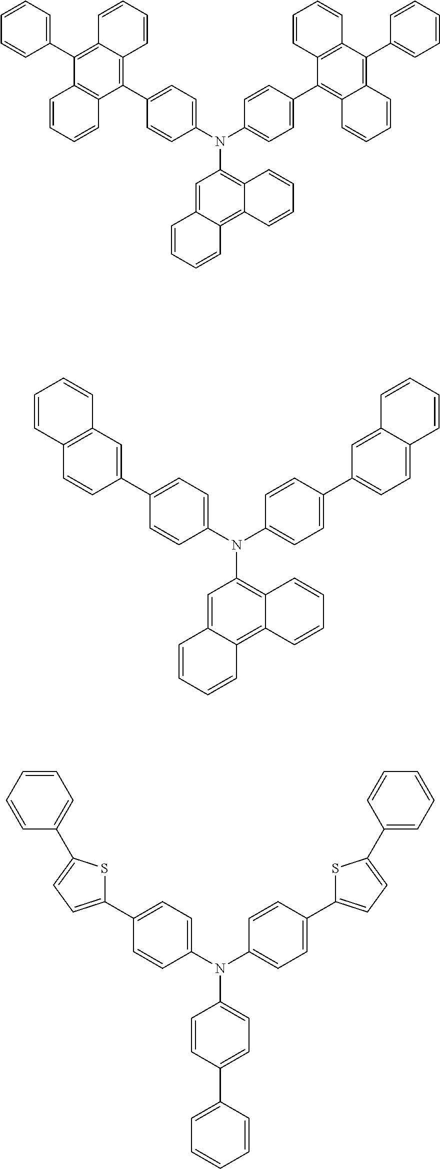 [SMILES: c1ccc(-c2c3ccccc3c(-c3ccc(N(c4ccc(-c5c6ccccc6c(-c6ccccc6)c6ccccc56)cc4)c4cc5ccccc5c5ccccc45)cc3)c3ccccc23)cc1 ; c1ccc2cc(-c3ccc(N(c4ccc(-c5ccc6ccccc6c5)cc4)c4cc5ccccc5c5ccccc45)cc3)ccc2c1 ; c1ccc(-c2ccc(N(c3ccc(-c4ccc(-c5ccccc5)s4)cc3)c3ccc(-c4ccc(-c5ccccc5)s4)cc3)cc2)cc1]

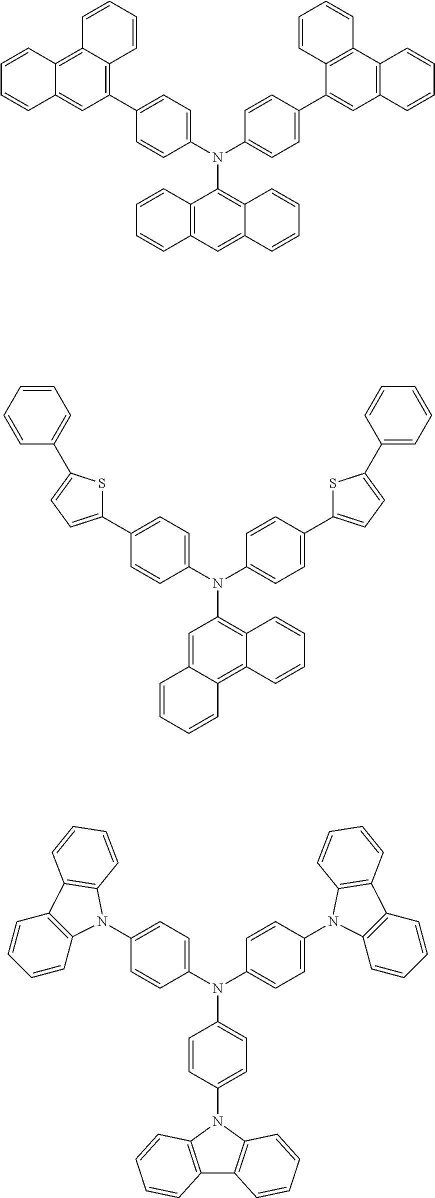 [SMILES: c1ccc2c(N(c3ccc(-c4cc5ccccc5c5ccccc45)cc3)c3ccc(-c4cc5ccccc5c5ccccc45)cc3)c3ccccc3cc2c1 ; c1ccc(-c2ccc(-c3ccc(N(c4ccc(-c5ccc(-c6ccccc6)s5)cc4)c4cc5ccccc5c5ccccc45)cc3)s2)cc1 ; c1ccc2c(c1)c1ccccc1n2-c1ccc(N(c2ccc(-n3c4ccccc4c4ccccc43)cc2)c2ccc(-n3c4ccccc4c4ccccc43)cc2)cc1]

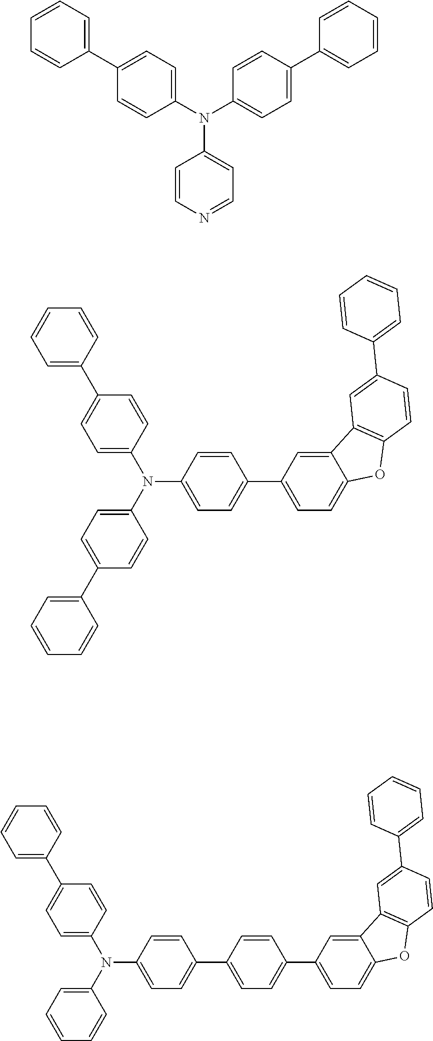 [SMILES: c1ccc(-c2ccc(N(c3ccncc3)c3ccc(-c4ccccc4)cc3)cc2)cc1 ; c1ccc(-c2ccc(N(c3ccc(-c4ccccc4)cc3)c3ccc(-c4ccc5oc6ccc(-c7ccccc7)cc6c5c4)cc3)cc2)cc1 ; c1ccc(-c2ccc(N(c3ccccc3)c3ccc(-c4ccc(-c5ccc6oc7ccc(-c8ccccc8)cc7c6c5)cc4)cc3)cc2)cc1]

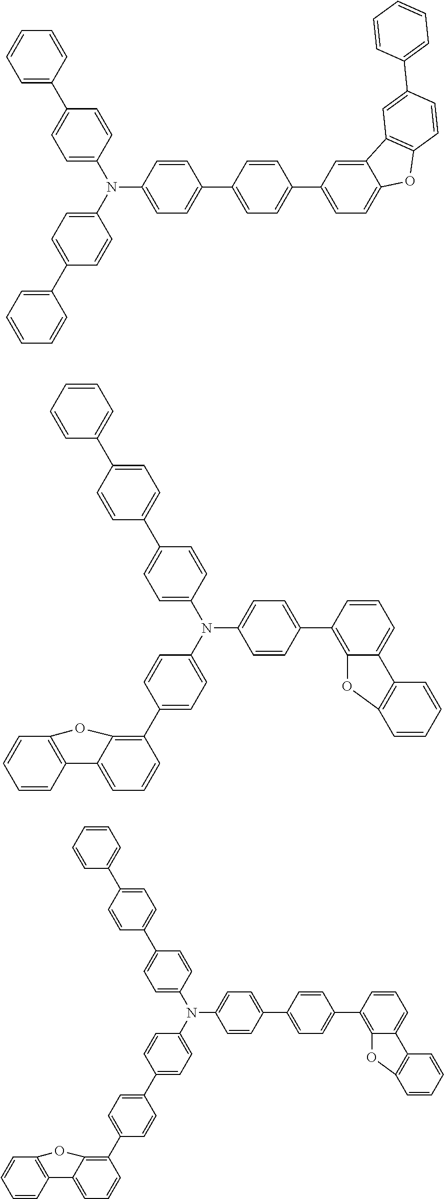

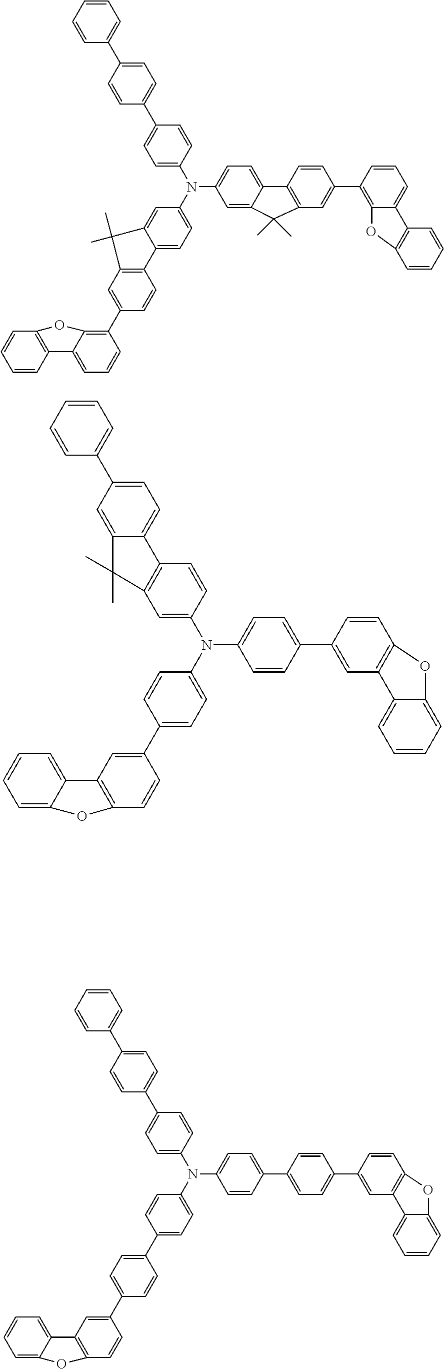

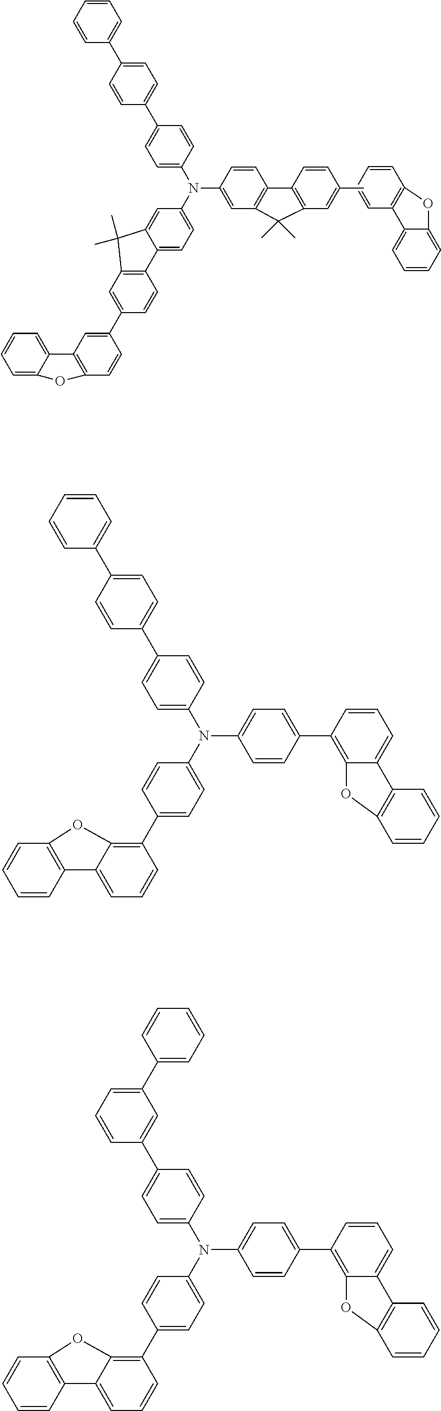

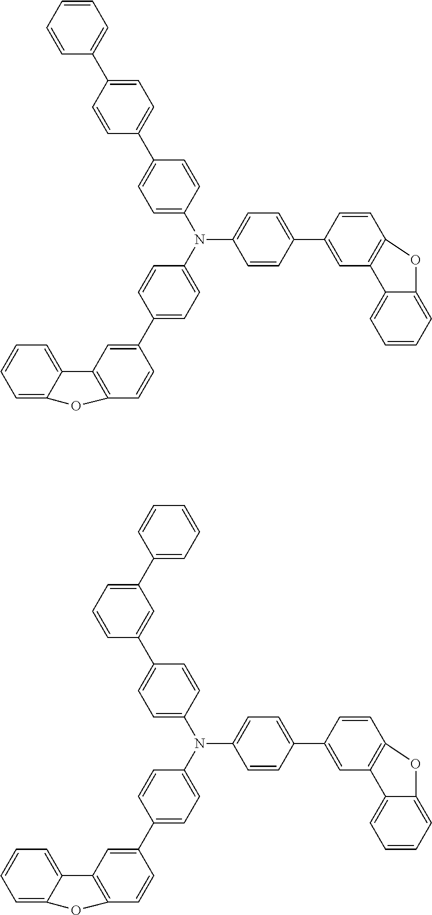 [SMILES: c1ccc(-c2ccc(-c3ccc(N(c4ccc(-c5ccc6oc7ccccc7c6c5)cc4)c4ccc(-c5ccc6oc7ccccc7c6c5)cc4)cc3)cc2)cc1 ; c1ccc(-c2cccc(-c3ccc(N(c4ccc(-c5ccc6oc7ccccc7c6c5)cc4)c4ccc(-c5ccc6oc7ccccc7c6c5)cc4)cc3)c2)cc1]

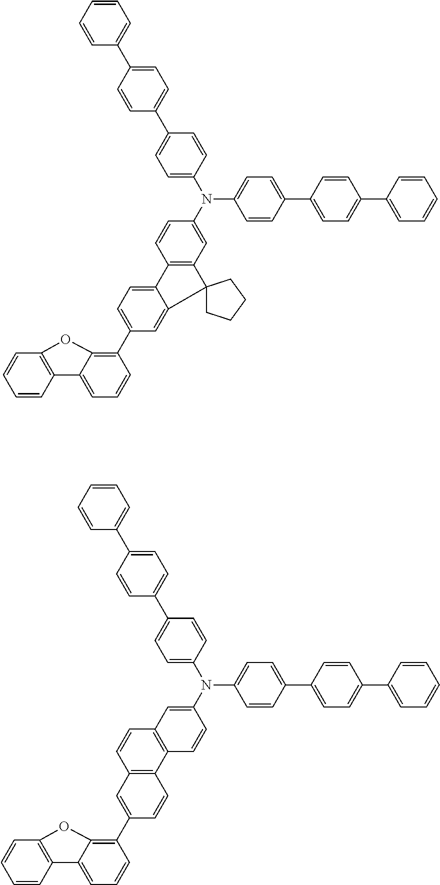 [SMILES: c1ccc(-c2ccc(-c3ccc(N(c4ccc(-c5ccc(-c6ccccc6)cc5)cc4)c4ccc5c(c4)C4(CCCC4)c4cc(-c6cccc7c6oc6ccccc67)ccc4-5)cc3)cc2)cc1 ; c1ccc(-c2ccc(-c3ccc(N(c4ccc(-c5ccc(-c6ccccc6)cc5)cc4)c4ccc5c(ccc6cc(-c7cccc8c7oc7ccccc78)ccc65)c4)cc3)cc2)cc1]

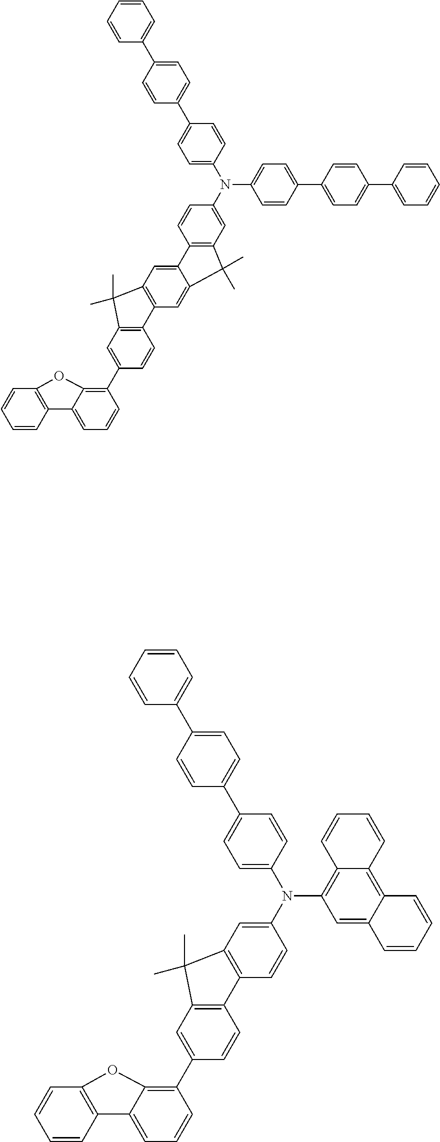 [SMILES: CC1(C)c2cc(-c3cccc4c3oc3ccccc34)ccc2-c2cc3c(cc21)-c1ccc(N(c2ccc(-c4ccc(-c5ccccc5)cc4)cc2)c2ccc(-c4ccc(-c5ccccc5)cc4)cc2)cc1C3(C)C ; CC1(C)c2cc(-c3cccc4c3oc3ccccc34)ccc2-c2ccc(N(c3ccc(-c4ccc(-c5ccccc5)cc4)cc3)c3cc4ccccc4c4ccccc34)cc21]

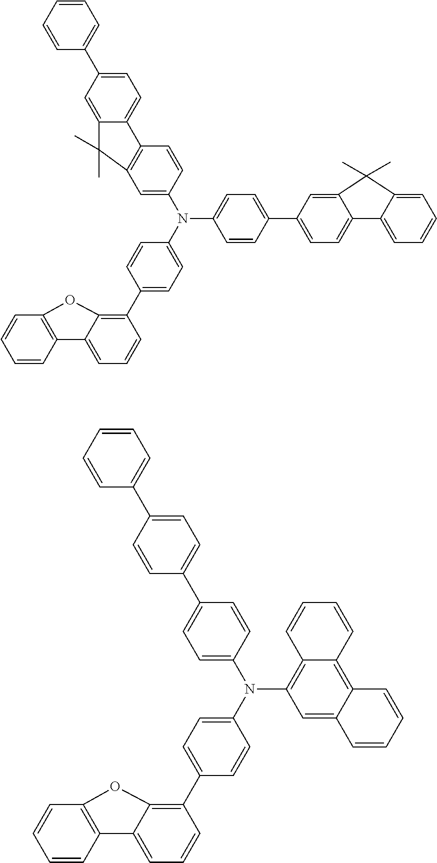 [SMILES: CC1(C)c2ccccc2-c2ccc(-c3ccc(N(c4ccc(-c5cccc6c5oc5ccccc56)cc4)c4ccc5c(c4)C(C)(C)c4cc(-c6ccccc6)ccc4-5)cc3)cc21 ; c1ccc(-c2ccc(-c3ccc(N(c4ccc(-c5cccc6c5oc5ccccc56)cc4)c4cc5ccccc5c5ccccc45)cc3)cc2)cc1]

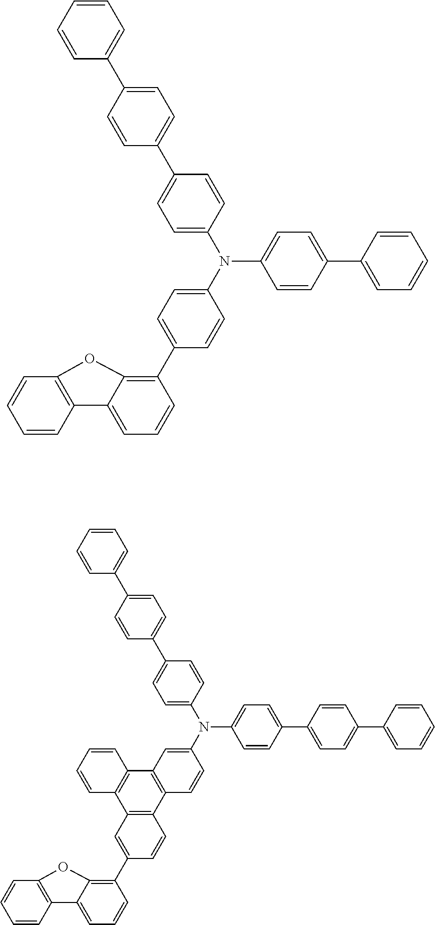 [SMILES: c1ccc(-c2ccc(-c3ccc(N(c4ccc(-c5ccccc5)cc4)c4ccc(-c5cccc6c5oc5ccccc56)cc4)cc3)cc2)cc1 ; c1ccc(-c2ccc(-c3ccc(N(c4ccc(-c5ccc(-c6ccccc6)cc5)cc4)c4ccc5c6ccc(-c7cccc8c7oc7ccccc78)cc6c6ccccc6c5c4)cc3)cc2)cc1]

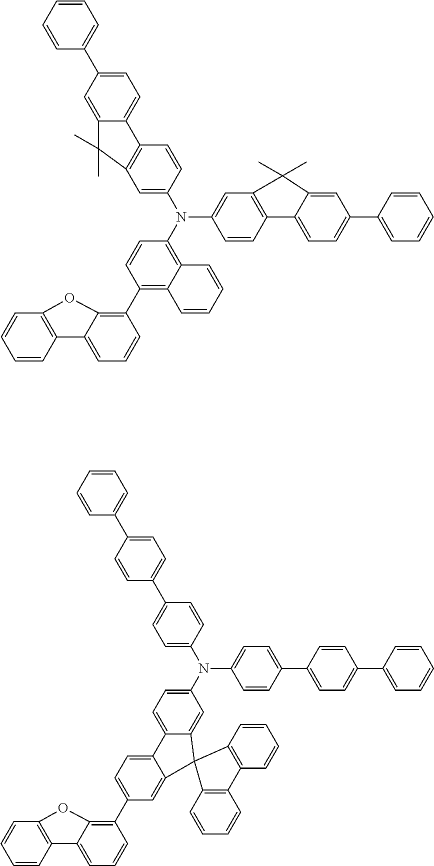 [SMILES: CC1(C)c2cc(-c3ccccc3)ccc2-c2ccc(N(c3ccc4c(c3)C(C)(C)c3cc(-c5ccccc5)ccc3-4)c3ccc(-c4cccc5c4oc4ccccc45)c4ccccc34)cc21 ; c1ccc(-c2ccc(-c3ccc(N(c4ccc(-c5ccc(-c6ccccc6)cc5)cc4)c4ccc5c(c4)C4(c6ccccc6-c6ccccc64)c4cc(-c6cccc7c6oc6ccccc67)ccc4-5)cc3)cc2)cc1]

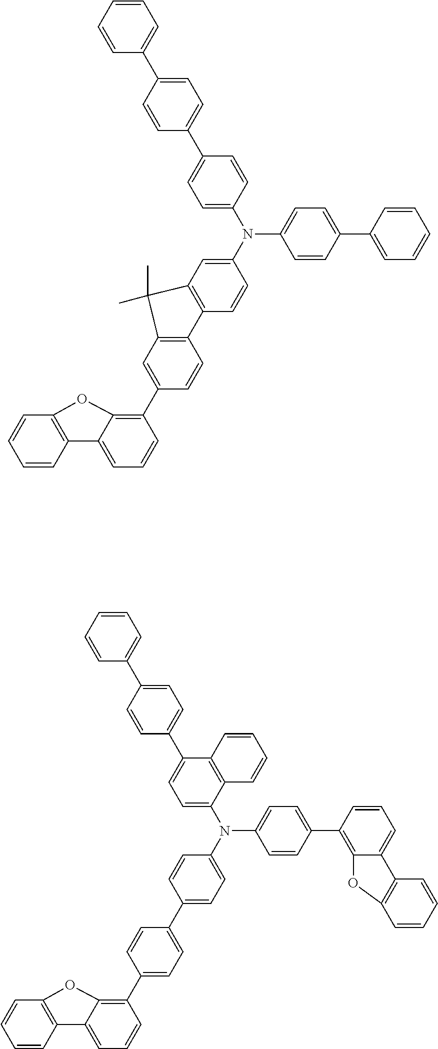 [SMILES: CC1(C)c2cc(-c3cccc4c3oc3ccccc34)ccc2-c2ccc(N(c3ccc(-c4ccccc4)cc3)c3ccc(-c4ccc(-c5ccccc5)cc4)cc3)cc21 ; c1ccc(-c2ccc(-c3ccc(N(c4ccc(-c5ccc(-c6cccc7c6oc6ccccc67)cc5)cc4)c4ccc(-c5cccc6c5oc5ccccc56)cc4)c4ccccc34)cc2)cc1]

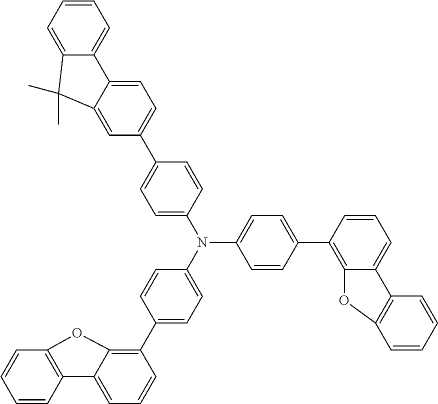 [SMILES: CC1(C)c2ccccc2-c2ccc(-c3ccc(N(c4ccc(-c5cccc6c5oc5ccccc56)cc4)c4ccc(-c5cccc6c5oc5ccccc56)cc4)cc3)cc21]

In addition, an aromatic tertiary amine compound and a styrylamine compound may be used in the hole transporting layer, which is selected from N,N,N′,N′-tetraphenyl-4,4′-diaminophenyl; N,N′-diphenyl-N,N′-bis(3-methylphenyl)-[1,1′-biphenyl]-4,4′-diamine (TPD); 2,2-bis(4-di-p-tolylaminophenyl)propane; 1,1-bis(4-di-p-tolylaminophenyl)cyclohexane; N,N,N′,N′-tetra-p-tolyl-4,4′-diaminobiphenyl; 1,1-bis(4-di-p-tolylaminophenyl)-4-phenylcyclohexane; bis(4-dimethylamino-2-methylphenyl)phenylmethane; bis(4-di-p-tolylaminophenyl)phenylmethane; N,N′-diphenyl-N,N′-di(4-methoxyphenyl)-4,4′-diaminobiphenyl; N,N,N′,N′-tetraphenyl-4,4′-diamino diphenyl ether; 4,4′-bis(diphenylamino)quadriphenyl; N,N,N-tri(p-tolyl)amine; 4-(di-p-tolylamino)-4′-[4-(di-p-tolylamino)styryl]stilbene; 4-N,N-diphenylamino-(2-diphenylvinyl)benzene; 3-methoxy-4′-N,N-diphenylaminostyrylbenezene; N-phenylcarbazole; a compound having two fused aromatic rings in its molecule, such as 4,4′-bis[N-(1-naphthyl)-N-phenylamino]biphenyl (NPD); and a starburst compound having three triphenylamine units, such as 4,4′,4″-tris[N-(3-methylphenyl)-N-phenylamino]triphenylamine (MTDATA).

In an embodiment of the invention, the hole transporting layer may be formed by using a composition for hole transporting layer which comprises a hole transporting material and a solvent.

The hole transporting material may be either of a macromolecule, such as a polymer, and a low molecular compound, such as a monomer. In view of the charge injection barrier, a compound having an ionization potential of 4.5 to 6.0 eV is preferred. Examples of such a hole transporting material include an aromatic amine derivative, a phthalocyanine derivative, a porphyrin derivative, an oligothiophene derivative, a polythiophene derivative, a benzylphenyl derivative, a compound wherein tertiary amines are linked via a fluorene group, a hydrazone derivative, a silazane derivative, a silanamine derivative, a phosphamine derivative, a quinacridone derivative, a polyaniline derivative, a polypyrrole derivative, a polyphenylene vinylene derivative, a polythienylene vinylene derivative, a polyquinoline derivative, a polyquinoxaline derivative, and carbon.

The derivative used herein includes, when using an aromatic amine derivative as an example, an aromatic amine itself and a compound wherein the main skeleton comprises an aromatic amine and may be a polymer or a monomer.

Of the above, in view of the amorphous nature and the visual light transmittance, preferred is an aromatic amine compound, with an aromatic tertiary amine compound being particularly preferred. The aromatic tertiary amine compound used herein is a compound having an aromatic tertiary amine structure and includes a compound having a substituent derived from an aromatic tertiary amine.

The aromatic tertiary amine compound is more preferably a macromolecular compound (a polymeric compound having repeating units) having a weight average molecular weight of 1,000 to 1,000,000 in view of obtaining a uniform emission due to smooth surface, although not particularly limited thereto. Preferred example thereof is a macromolecular compound having the following repeating unit represented by formula (I):

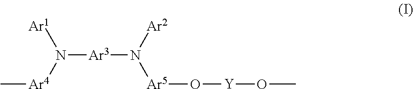

wherein
Ar¹ and Ar² each independently represent a substituted or unsubstituted aromatic hydrocarbon group or a substituted or unsubstituted aromatic heterocyclic group; Ar³ to Ar⁵ each independently represent a substituted or unsubstituted aromatic hydrocarbon group or a substituted or unsubstituted aromatic heterocyclic group; two groups bonded to the same nitrogen atom selected from Ar¹ to Ar⁵ may be bonded to each other to form a ring; and Y represents a linking group selected from the following groups:

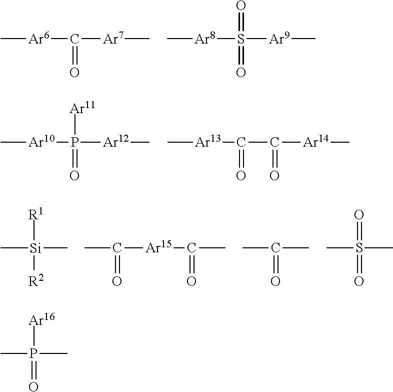

wherein Ar⁶ to Ar¹⁶ each independently represent a substituted or unsubstituted aromatic hydrocarbon group or a substituted or unsubstituted aromatic heterocyclic group; and R¹ and R² each independently represent a hydrogen atom or a substituent.

In view of the solubility, heat resistance, and hole injecting/transporting ability of the macromolecular compound, the aromatic hydrocarbon group and the aromatic heterocyclic group for Ar¹ to Ar¹⁶ is preferably a group having a ring selected from a benzene ring, a naphthalene ring, a phenanthrene ring, a thiophene ring, and a pyridine ring and more preferably a group having a ring selected from a benzene ring and a naphthalene ring.

The optional substituent of the aromatic hydrocarbon group and the aromatic heterocyclic group for Ar¹ to Ar¹⁶ has a molecular weight of generally 400 or less and preferably about 250 or less. The substituent is preferably an alkyl group, an alkenyl group, an alkoxy group, an aromatic hydrocarbon group, or an aromatic heterocyclic group.

The substituent represented by R¹ and R² may include an alkyl group, an alkenyl group, an alkoxy group, a silyl group, a siloxy group, an aromatic hydrocarbon group, and an aromatic heterocyclic group.

A polythiophene derivative, such as an electroconductive polymer (PEDOT/PSS) obtained by polymerizing 3,4-ethylenedioxythiophene in a high molecular weight polystyrenesulfonic acid, is also preferred as the hole transporting material. The terminal ends of this polymer may be capped with a methacrylate.

The concentration of the hole transporting material in the composition for hole transporting layer is arbitrary and, in view of uniform thickness of film, generally 0.01% by mass or more, preferably 0.1% by mass or more, more preferably 0.5% by mass or more, and generally 70% by mass or less, preferably 60% by mass or less, and more preferably 50% by mass or less. Within the above ranges, uneven thickness of film and defect in the hole transporting layer can be avoided.

The composition for hole transporting layer may contain an electron-accepting compound.

The electron-accepting compound is preferably a compound having an oxidation ability to receive one electron from the hole transporting material and more preferably a compound having an electron affinity of 4 eV or more, preferably 5 eV or more.

Examples of such an electron-accepting compound include at least one compound selected from the group consisting of a triarylboron compound, a metal halide, a Lewis acid, an organic acid, an onium salt, a salt between an arylamine and a metal halide, and a salt between an arylamine and a Lewis acid. More specific examples include an onium salt having an organic group, such as 4-isopropyl-4′-methyldiphenyliodonium tetrakis(pentafluorophenyl)borate and triphenylsulfonium tetrafluoroborate; iron(III) chloride; a high valence inorganic compound, such as ammonium peroxodisulfate; a cyano compound, such as tetracyanoethylene; an aromatic boron compound, such as tris(pentafluorophenyl)borane; a fullerene derivative; iodine; and a sulfonate ion, such as polystyrenesulfonate ion, alkylbenzenesulfonate ion, and camphorsulfonate ion.

These electron-accepting compounds improve the electroconductivity of the hole transporting layer by oxidizing the hole transporting material.

The content of the electron-accepting compound to the hole transporting material in the composition for hole transporting layer is generally 0.1 mol % or more, preferably 1 mol % or more, and generally 100 mol % or less, preferably 40 mol % or less.

In addition to the hole transporting material and the electron-accepting compound, the composition for hole transporting layer may contain other component, such as a light emitting material, an electron transporting material, a binder resin, and a coating improver, which may be used alone or in combination of two or more in an arbitrary ratio.

In an embodiment of the invention, a hole transporting material suitable for use in a coating method is preferably used. Examples of such a hole transporting material include polyvinylcarbazole and its derivative, polysilane and its derivative, polysiloxane derivative having an aromatic amine residue in its side chain or main chain, pyrazoline derivative, an arylamine derivative, stilbene derivative, triphenyldiamine derivative, polyaniline and its derivative, polythiophene and its derivative, polypyrrole and its derivative, polyarylamine and its derivative, poly(p-phenylenevinylene) and its derivative, polyfluorene derivative, a mocromolecular compound having an aromatic amine residue, and poly(2,5-thienylenevinylene) and its derivative.

The hole transporting material is preferably a macromolecular compound, for example, a polymer. By using a macromolecular compound, the film-forming properties are improved and a uniform emission of organic EL device is obtained. The number average molecular weight of such a hole transporting material is 10,000 or more, preferably 3.0×10⁴ to 5.0×10⁵, and more preferably 6.0×10⁴ to 1.2×10⁵ when calibrated with a standard polystyrene. The weight average molecular weight of the hole transporting material is 1.0×10⁴ or more, preferably 5.0×10⁴ to 1.0×10⁶, and more preferably 1.0×10⁵ to 6.0×10⁵.

Such a hole transporting material is preferably a mocromolecular compound, such as polyvinylcarbazole and its derivative, polysilane and its derivative, polysiloxane derivative having an aromatic amine residue in its side chain or main chain, polyaniline and its derivative, polythiophene and its derivative, polyfluorene derivative, a mocromolecular compound having an aromatic amine residue, poly(p-phenylenevinylene) and its derivative, and poly(2,5-thienylenevinylene) and its derivative, with polyvinylcarbazole and its derivative, polysilane and its derivative, polysiloxane derivative having an aromatic amine residue in its side chain or main chain, polyfluorene derivative, and a mocromolecular compound having an aromatic amine residue being more preferred. A low molecular hole transporting material is used preferably by dispersing into a macromolecular binder.

Polyvinylcarbazole and its derivative is obtained, for example, by a cation polymerization or a radical polymerization of a vinyl monomer.

Since the siloxane structure is little hole transporting, a residue of the low molecular hole transporting material mentioned above is introduced into the side chain or main chain of polysiloxane and its derivative. A compound having a residue of a hole transporting aromatic amine in its side chain or main chain is particularly preferred.

A polymer comprising a fluorenediyl unit represented by formula (Z) is also preferred as the hole transporting material. When this polymer is used in the hole transporting layer of organic EL device in contact with an organic compound having a fused ring or more than one aromatic ring, the efficiency of hole injection is enhanced and the current density at driving is large.

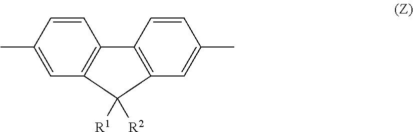

In formula (Z), R¹ and R² may be the same or different and each independently represent a hydrogen atom, an alkyl group, an alkoxy group, an aryl group, or a monovalent heterocyclic group. The alkyl group has 1 to 10 carbon atoms. The alkoxy group has 1 to 10 carbon atoms. Examples of the aryl group include a phenyl group and a naphthyl group. Example of the monovalent heterocyclic group include a pyridyl group. The aryl group and the monovalent heterocyclic group may have a substituent. In view of improving the solubility of the macromolecular compound, the substituent is preferably an alkyl group having 1 to 10 carbon atoms and an alkoxy group having 1 to 10 carbon atoms.

In formula (Z), then aryl group and the monovalent heterocyclic group may have a crosslinkable group, such as a vinyl group, an ethynyl group, a butenyl group, an acryl-containing group, an acrylate-containing group, an acrylamide-containing group, a methacryl-containing group, a methacrylate-containing group, a methacrylamide-containing group, a vinyl ether-containing group, a vinylamino group, a silanol-containing group, and a group containing a small-membered ring, for example, cyclopropane, cyclobutane, epoxide, oxetane, diketene, and episulfide.

Preferred examples of the fluorenediyl unit are shown below:

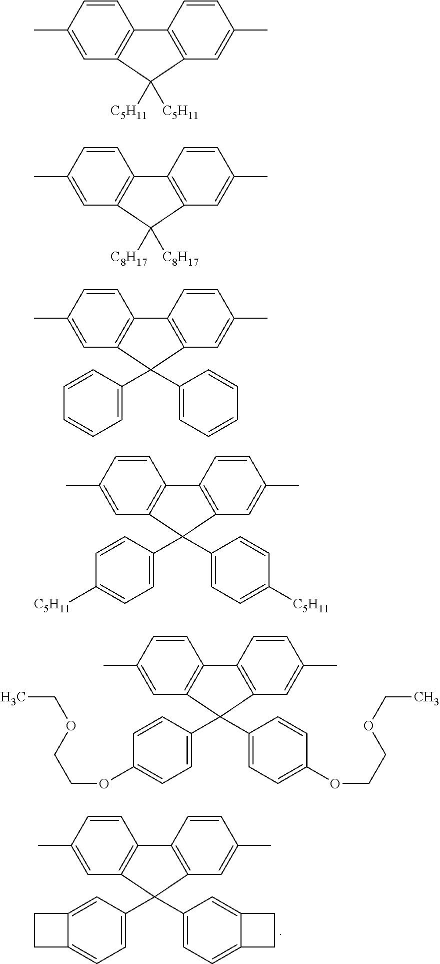

A polymer, for example, a polyarylamine having a repeating unit comprising the above fluorenediyl unit and an aromatic tertiary amine compound unit is particularly preferred as the hole transporting material.

Example of the aromatic tertiary amine compound unit includes a repeating unit represented by formula (K):

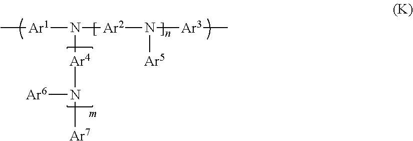

wherein Ar¹, Ar², Ar³, and Ar⁴ each independently represent an arylene group or a divalent heterocyclic group, Ar⁵, Ar⁶ and Ar⁷ each independently represent an aryl group or a monovalent heterocyclic group, Ar⁶ and Ar⁷ may form a ring together with the nitrogen atom to which Ar^{c} and Ar⁷ are bonded, and m and n each independently represent 0 or 1.

Example of the arylene group includes a phenylene group, and example of the divalent heterocyclic group includes a pyridinediyl group. These groups may have a substituent

Examples of the aryl group include a phenyl group and a naphthyl group. Example of the monovalent heterocyclic group includes a pyridyl group. These groups may have a substituent.

Examples of the monovalent heterocyclic group include a thienyl group, a furyl group, and a pyridyl group.

In view of the solubility of the macromolecular compound, the optional substituent for the arylene group, the aryl group, the divalent heterocyclic group, and the monovalent heterocyclic group is preferably an alkyl group, an alkoxy group, and an aryl group, with an alkyl group being more preferred. The alkyl group has 1 to 10 carbon atoms and the alkoxy group has 1 to 10 carbon atoms. Examples of the aryl group include a phenyl group and a naphthyl group.

The substituent may include a crosslinkable group, such as a vinyl group, an ethynyl group, a butenyl group, an acryl-containing group, an acrylate-containing group, an acrylamide-containing group, a methacryl-containing group, a methacrylate-containing group, a methacrylamide-containing group, a vinyl ether-containing group, a vinylamino group, a silanol-containing group, and a group containing a small-membered ring, for example, cyclopropane, cyclobutane, epoxide, oxetane, diketene, and episulfide.

In formula (K), Ar¹, Ar², Ar³, and Ar⁴ are each preferably an arylene group and more preferably a phenylene group. Ar⁵, Ar⁶ and Ar⁷ are each preferably an aryl group and more preferably a phenyl group.

The carbon atom in Ar² and the carbon atom in Ar³ may be bonded to each other directly or via a divalent group, such as —O— and —S—.

In view of easily synthesizing the monomer, m and n are each preferably 0.

Examples of the repeating unit represented by formula (K) include the following repeating units:

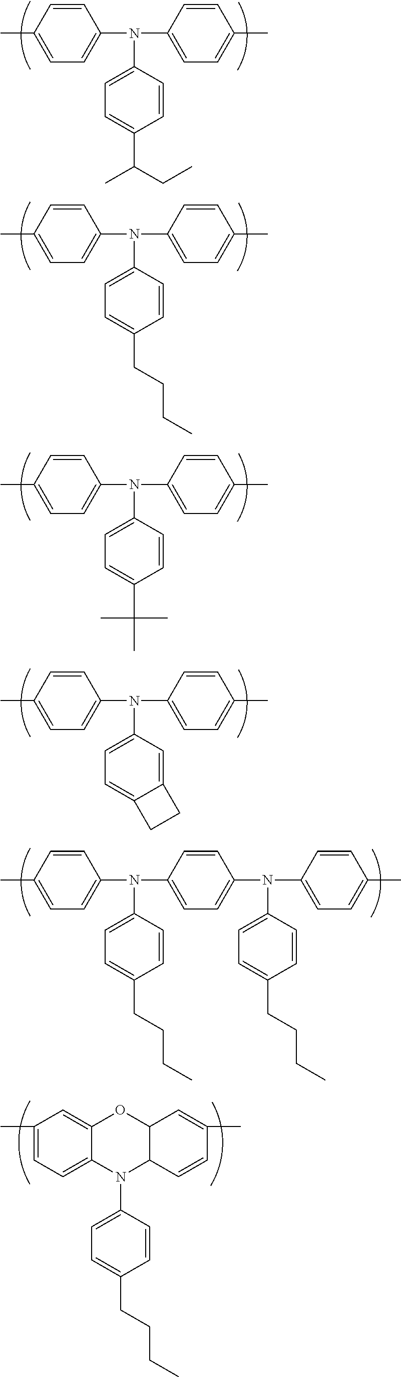

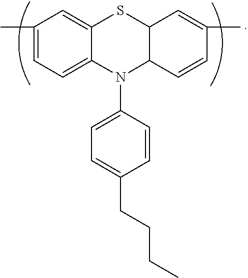

When the hole transporting material has no crosslinkable group, a crosslinking agent having a crosslinkable group is preferably used. Example of the crosslinking agent includes a compound having a polymerizable group selected from the group consisting of a vinyl group, an acetyl group, a butenyl group, an acryl group, an acrylamido group, an methacryl group, an methacrylamido group, a vinyl ether group, a vinylamino group, a silanol group, a cyclopropyl group, a cyclobutyl group, an epoxy group, an oxetane group, a diketone group, an episulfide group, a lactone group, and a lactam group. Preferred as the crosslinking agent is a polyfunctional acrylate, such as dipentaerythritol hexaacrylate (DPHA) and trispentaerythritol octaacrylate (TPEA).

By using the material having a crosslinkable group or the crosslinking agent, the underlayer (hole transporting layer) is prevented from being dissolved in the solvent for forming the upper layer even when another functional layer (upper layer) is formed on the underlayer by a coating method.

In an embodiment of the invention, a hole transporting material having a hole transporting portion and a crosslinkable group is also preferably used. The hole transporting portion may include a triarylamine structure; an aromatic ring structure having three or more rings, such as a fluorene ring, an anthracene ring, a pyrene ring, a carbazole ring, a dibenzofuran ring, a dibenzothiophene ring, a phenoxazine ring, and a phenanthroline ring; an aromatic heterocyclic structure, such as a thiophene ring and a silole ring; and a metal complex structure.

Of the above, in view of improving the electrochemical stability and the hole transporting ability, the triarylamine structure is preferred as the hole transporting portion.

In addition, the hole transporting portion is preferably a polymer because it easily becomes insoluble in an organic solvent by crosslinking, and a polymer having a repeating unit represented by formula (L) is particularly preferred in view of improving the electrochemical stability and the hole transporting ability:

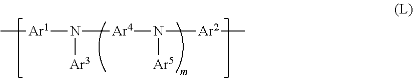

wherein m represents an integer of 0 to 3; Ar¹ and Ar² each independently represent a single bond, a substituted or unsubstituted aromatic hydrocarbon group, or a substituted or unsubstituted aromatic heterocyclic group; and Ar³ to Ar⁵ each independently represent a substituted or unsubstituted aromatic hydrocarbon group or a substituted or unsubstituted aromatic heterocyclic group; provided that Ar¹ and Ar² do not represent a single bond simultaneously.

The aromatic hydrocarbon group may include, for example, a six-membered monocyclic group or a monovalent fused ring group having 2 to 5 six-membered rings, such as a benzene ring, a naphthalene ring, an anthracene ring, a phenanthrene ring, a perylene ring, a tetracene ring, a pyrene ring, a benzopyrene ring, a chrysene ring, a triphenylene ring, an acenaphthene ring, a fluoranthene ring, and a fluorene ring.

The aromatic heterocyclic group may include, for example, a five- or six-membered monocyclic group or a monovalent fused ring group having 2 to 4 five- or six-membered rings, such as a furan ring, a benzofuran ring, a thiophene ring, a benzothiophene ring, a pyrrole ring, a pyrazole ring, an imidazole ring, an oxadiazole ring, an indole ring, a carbazole ring, a pyrroloimidazole ring, a pyrrolopyrazole ring, a pyrrolopyrrole ring, a thienopyrrole ring, a thienothiophene ring, a furopyrrole ring, a furofuran ring, a thienofuran ring, a benzisoxazole ring, a benzisothiazole ring, a benzimidazole ring, a pyridine ring, a pyrazine ring, a pyridazine ring, a pyrimidine ring, a triazine ring, a quinoline ring, an isoquinoline ring, a cinnoline ring, a quinoxaline ring, a phenanthridine ring, a benzimidazole ring, a perimidine ring, a quinazoline ring, a quinazoline ring, and an azulene ring.

In view of the solubility to a solvent and the heat resistance, Ar¹ to Ar⁵ each independently and preferably represent a monovalent group of a ring selected from the group consisting of a benzene ring, a naphthalene ring, an anthracene ring, a phenanthrene ring, a triphenylene ring, a pyrene ring, a thiophene ring, a pyridine ring, and a fluorene ring.

A group wherein one or more kinds of rings selected from the above group are linked via a single bond is also preferred as Ar¹ to Ar⁵, with a biphenyl group, a biphenylene group, a terphenyl group, and a terphenylene group being more preferred.

The optional group of the aromatic hydrocarbon group and the aromatic heterocyclic group may include a linear, branched or cyclic alkyl group having 1 to 24, preferably 1 to 12 carbon atoms, such as a methyl group, an ethyl group, a n-propyl group, an isopropyl group, a n-butyl group, an isobutyl group, a sec-butyl group, a tert-butyl group, a n-hexyl group, a cyclohexyl group, and a dodecyl group; an alkenyl group having 2 to 24, preferably 2 to 12 carbon atoms, such as a vinyl group; an alkynyl group having 2 to 24, preferably 2 to 12 carbon atoms, such as an ethynyl group; an alkoxy group having 1 to 24, preferably 1 to 12 carbon atoms, such as a methoxy group and an ethoxy group; an aryloxy group having 4 or more, preferably 5 or more and 36 or less, preferably 24 or less carbon atoms, such as a phenoxy group, a naphthoxy group, and a pyridyloxy group; an alkoxycarbonyl group having 2 to 24, preferably 2 to 12 carbon atoms, such as a methoxycarbonyl group and an ethoxycarbonyl group; a dialkylamino group having 2 to 24, preferably 2 to 12 carbon atoms, such as a dimethylamino group and a diethylamino group; a diarylamino group having 10 or more, preferably 12 or more and 36 or less, preferably 24 or less carbon atoms, such as a diphenylamino group, a ditolylamino group, and a N-carbazolyl group; an arylalkylamino group having 7 to 36, preferably 7 to 24 carbon atoms, such as a phenylmethylamino group; an acyl group having 2 to 24, preferably 2 to 12 carbon atoms, such as an acetyl group and a benzoyl group; a halogen atom, such as a fluorine atom and a chlorine atom; a haloalkyl group having 1 to 12, preferably 1 to 6 carbon atoms, such as a trifluoromethyl group; an alkylthio group having 1 to 24, preferably 1 to 12 carbon atoms, such as a methylthio group and an ethylthio group; an arylthio group having 4 or more, preferably 5 or more and 36 or less, preferably 24 or less carbon atoms, such as a phenylthio group, a naphthylthio group, and a pyridylthio group; a silyl group having 2 or more, preferably 3 or more and 36 or less, preferably 24 or less carbon atoms, such as a trimethylsilyl group and a triphenylsilyl group; a siloxy group having 2 or more, preferably 3 or more and 36 or less, preferably 24 or less carbon atoms, such as a trimethylsiloxy group and a triphenylsiloxy group; a cyano group; an aromatic hydrocarbon group having 6 to 36, preferably 6 to 24 carbon atoms, such as a phenyl group and a naphthyl group; and an aromatic heterocyclic group having 3 or more, preferably 4 or more and 36 or less, preferably 24 or less carbon atoms, such as a thienyl group and a pyridyl group.

Of the above optional substituents, an alkyl group having 1 to 12 carbon atoms and an alkoxy group having 1 to 12 carbon atoms are preferred in view of the solubility.

Each of the above optional substituents may further have a substituent which is selected from the optional substituents mentioned above.

The number of carbon atoms of Ar¹ to Ar⁵ inclusive of the carbon atoms in the substituent is 3 or more, preferably 5 or more, and more preferably 6 or more, and 72 or less, preferably 48 or less, and more preferably 25 or less.

In formula (L), m is an integer of 0 to 3 and preferably m is 0 because the film-forming properties are improved. In view of improving the hole transporting ability, m is preferably 1 to 3.

When m is 2 or more, two or more groups Ar⁴ and two or more groups Ar⁵ may be the same or different, respectively. Groups Ar⁴ and groups Ar⁵ may be bonded to each other directly or via a linking group, respectively to form a ring structure.

When the hole transporting material includes a crosslinkable group, the solubility to a solvent is largely changed before and after the reaction (insolubilization) caused by exposing to heat and/or an active energy ray.

The crosslinkable group used herein is a group which reacts with the same of different group in another molecule in the vicinity thereof by exposing to heat and/or an active energy ray, thereby forming a new chemical bond.

For example, the following crosslinkable groups are easily insolubilized:

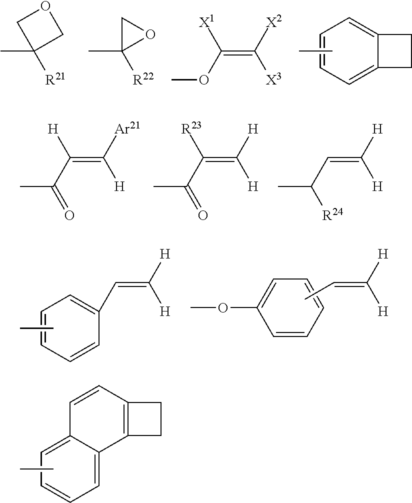

wherein R²¹ to R²³ each independently represent a hydrogen atom or a substituted or unsubstituted alkyl group;

Ar²¹ represents a substituted or unsubstituted aromatic group;

X¹, X² and X³ each independently represents a hydrogen atom or a halogen atom; and

R²⁴ represents a hydrogen atom or a vinyl group.

The benzocyclobutene ring may have a substituent and the substituents may be bonded to each other to form a ring.

The alkyl group for R²¹ to R²³ may include an alkyl group having 1 to 24, preferably 1 to 12 carbon atoms, such as a methyl group and an ethyl group.

Example of the aromatic group for Ar²¹ is the same as those described above with respect to Ar¹ to Ar⁵.

The optional substituent for R²¹ to R²³ and Ar²¹ is not particularly limited and, for example, selected from those mentioned above.

A group which is insolubilized by a cation polymerization, for example, a cyclic ether group, such as an epoxy group and an oxetane group, and a vinyl ether group, is preferred as the crosslinkable group because such a group is highly reactive and easily insolubilized. The oxetane group is particularly preferred because the rate of cation polymerization is easily controlled and the vinyl ether group is particularly preferred because a hydroxyl group which may damage a device during the cation polymerization is difficult to be formed.

A group capable of a cycloaddition, for example, an arylvinylcarbonyl group, such as a cinnamoyl, and a group having a benzocyclobutene ring, is also preferred in view of further enhancing the electrochemical stability.

A group having a benzocyclobutene ring is particularly preferred because the structure after insolubilization is very stable.

For example, a group represented by formula (M) is preferred:

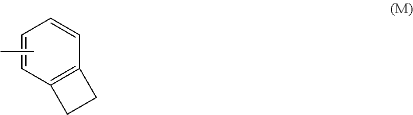

wherein the benzocyclobutene ring may have a substituent and the substituents may be bonded to each other to form a ring.

The crosslinkable group may be bonded to a mono- or di-valent aromatic group in the molecule directly or via a divalent group. The divalent group preferably comprises 1 to 30 groups selected from —O—, —C(═O)—, and —CH₂— wherein the hydrogen atom may be substituted, which are linked together in an arbitrary order. Examples of the crosslinkable group to be bonded via a divalent group are shown below, although not limited thereto.

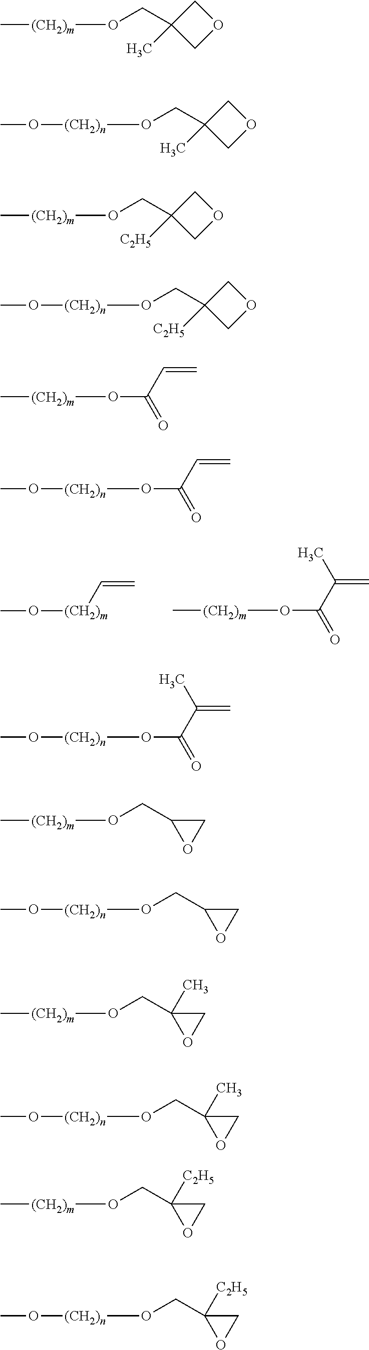

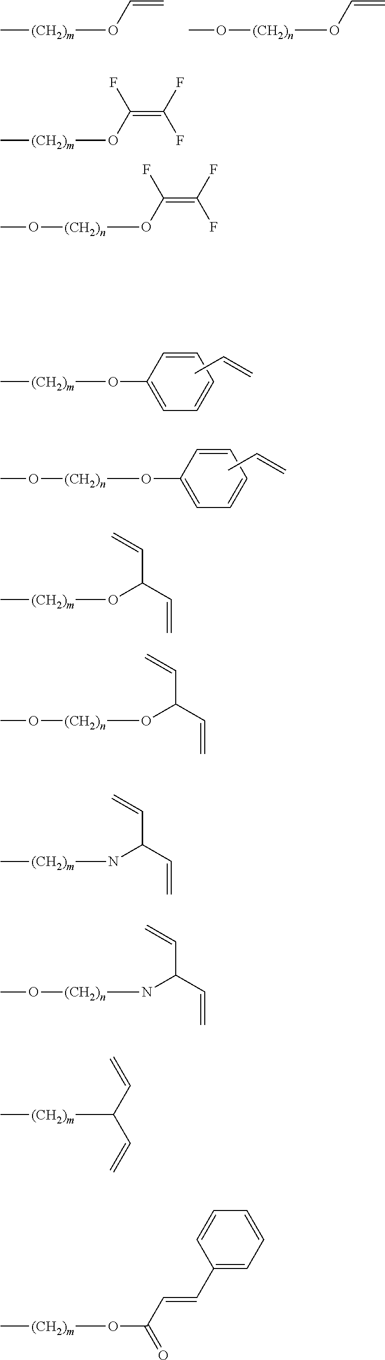

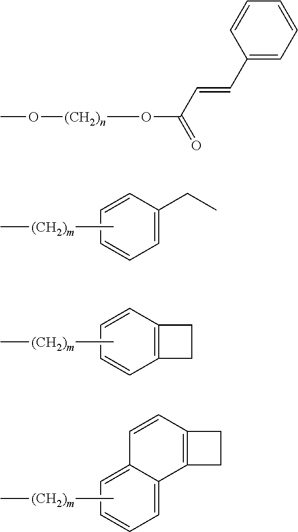

In the above formulae, m is an integer of 0 to 12 and n is an integer of 1 to 12.

Other examples of the group having a crosslinkable group are shown below.

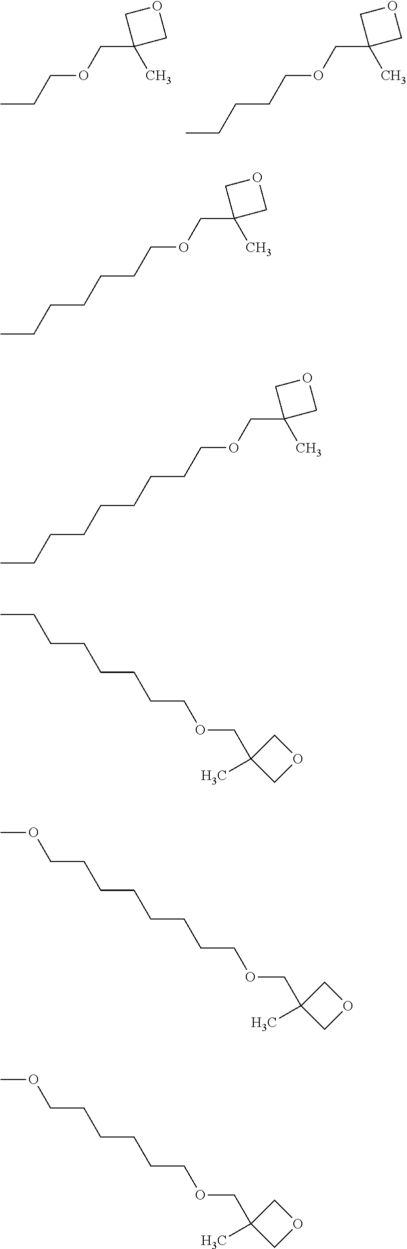

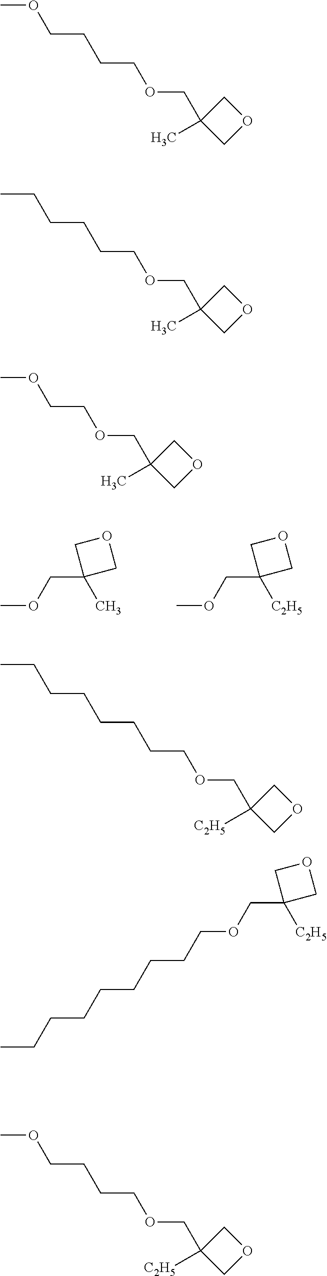

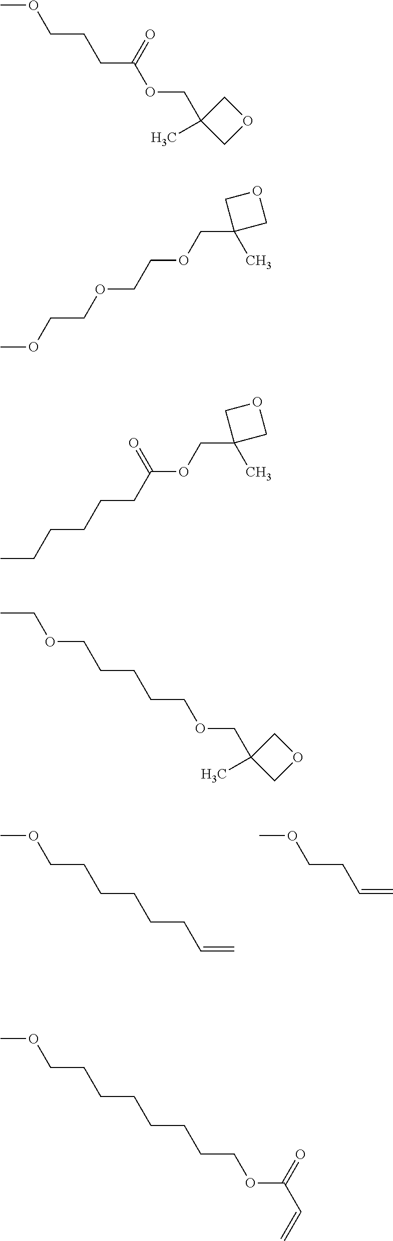

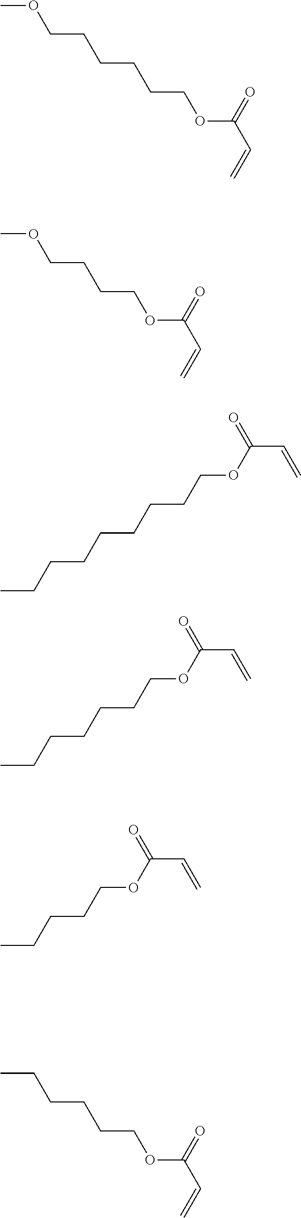

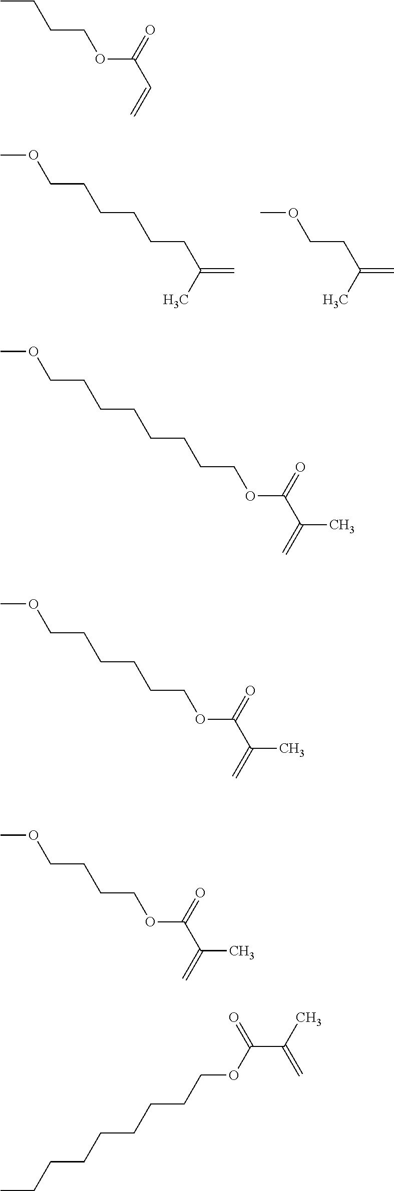

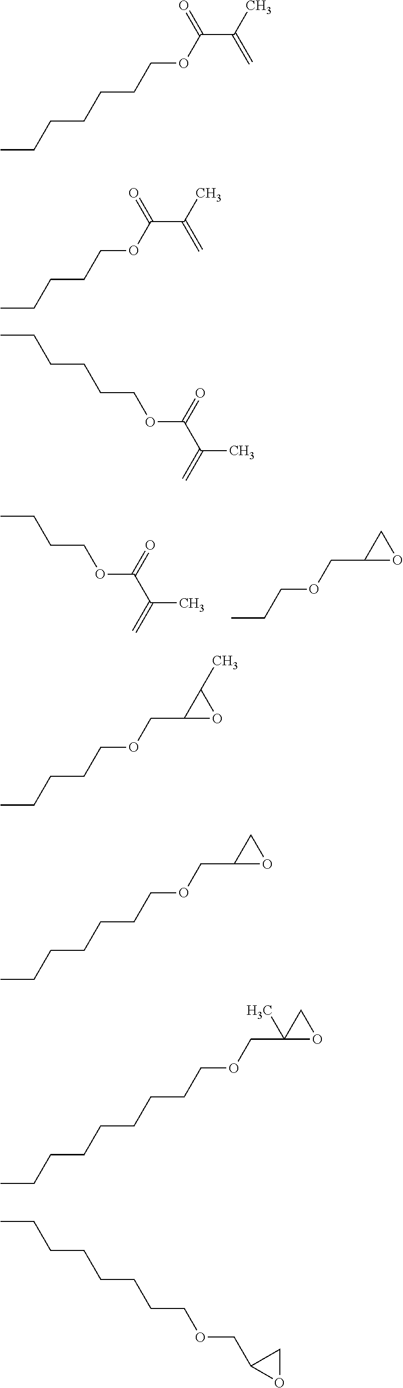

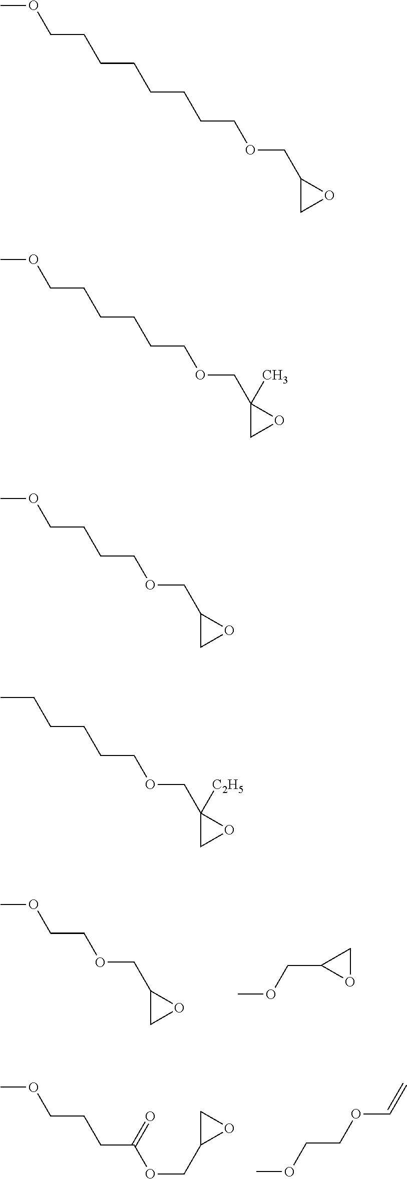

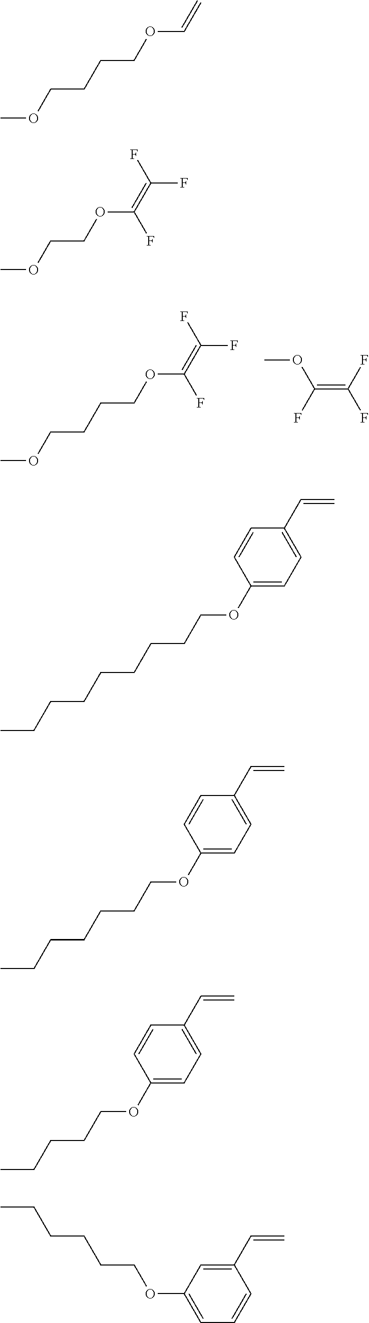

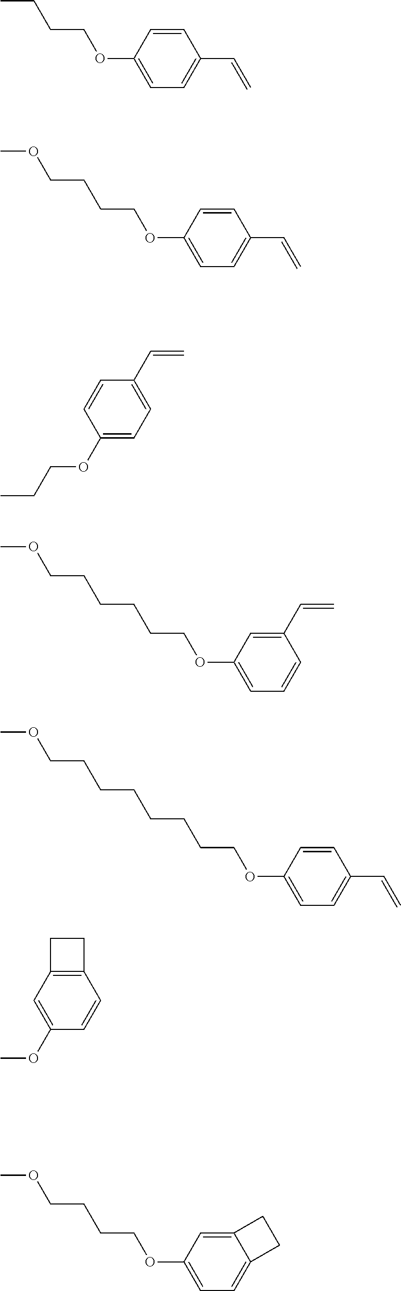

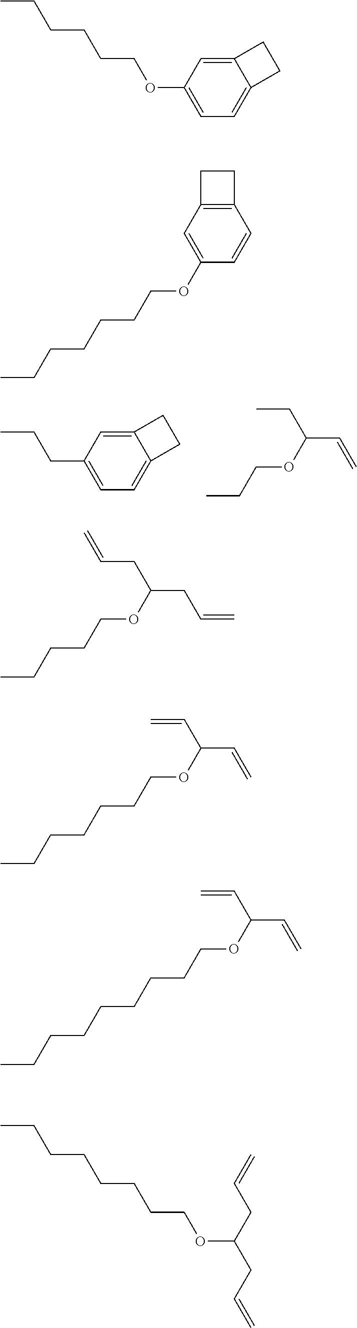

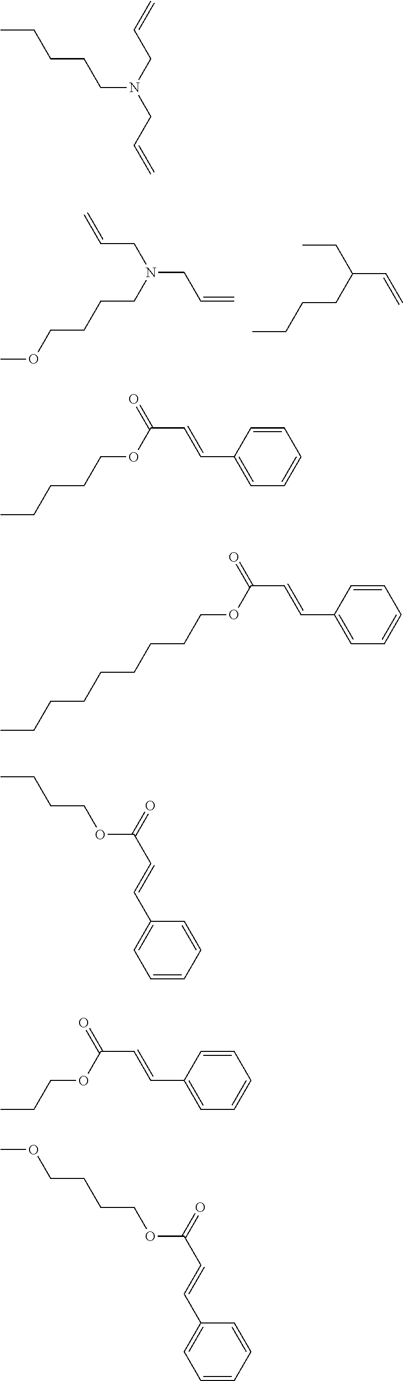

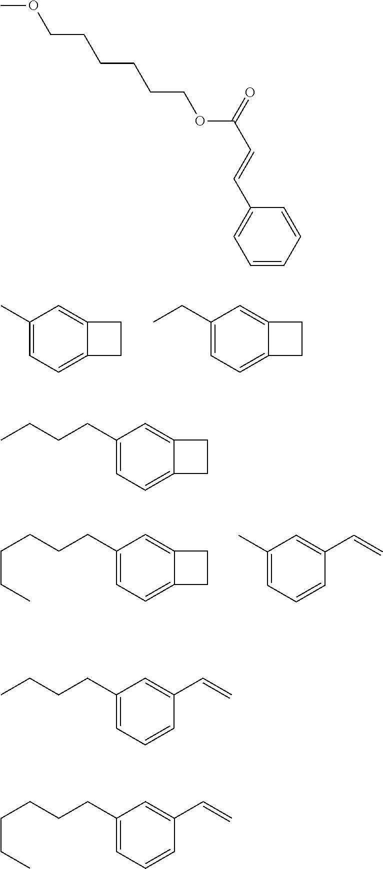

In an embodiment of the invention, the hole transporting material preferably comprises an electroconductive polymer or oligomer. The electroconductive polymer or oligomer is generally a mixture of an electron-donating compound, an electron-accepting compound, or an acidic compound. The mixture may be a solid or a liquid, preferably a solution, a dispersion, a colloid, an ink, or a varnish, because these are suitable for forming a solid layer by a coating method. The mixture may be contain an additive to improve the hole transporting ability and the film-forming properties.

Examples of the electroconductive polymer or oligomer usable in an embodiment of the invention will be described below.

Representative examples of the electron-donating compound include an aromatic amine derivative, a phthalocyanine derivative, a porphyrin derivative, a thiophene derivative, a benzylphenyl derivative, a compound wherein tertiary amines are linked via a fluorene group, a hydrazone derivative, a silazane derivative, a silanamine derivative, a phosphamine derivative, a quinacridone derivative, an aniline derivative, a pyrrole derivative, a phenylenevinylene derivative, a thienylenevinylene derivative, a quinoline derivative, a quinoxaline derivative, and carbon. These derivatives may be any of a low molecular compound having a molecular weight of less than 1,000, an oligomer or a dendrimer having a molecular weight of 1,000 to 10,000, and a macromolecular compound having a molecular weight of 10,000 or more. Of the above, an aromatic amine derivative, a polythiophene derivative, a polyaniline derivative, and an oligoaniline derivative are preferably used.

Representative example of the electron-accepting compound and the acidic compound may be at least one compound selected from the group consisting of a triarylboron compound, a metal halide, a Lewis acid, an organic acid, an onium salt, a salt between an arylamine and a metal halide, and a salt between an arylamine and a Lewis acid. More specifically, examples thereof include an onium salt having an organic group, such as 4-isopropyl-4′-methyldiphenyliodonium tetrakis(pentafluorophenyl)borate and triphenylsulfonium tetrafluoroborate; iron(III) chloride; a high valence inorganic compound, such as ammonium peroxodisulfate; a cyano compound, such as tetracyanoethylene; an aromatic boron compound, such as tris(pentafluorophenyl)borane; a fullerene derivative; iodine; and a sulfonate ion, such as polystyrenesulfonate ion, alkylbenzenesulfonate ion, and camphorsulfonate ion.

Like the electron-donating compound, these derivatives may be any of a low molecular compound having a molecular weight of less than 1,000, an oligomer or a dendrimer having a molecular weight of 1,000 to 10,000, and a polymer having a molecular weight of 10,000 or more.

These electron-accepting compounds increase the electroconductivity of the hole transporting layer by oxidizing the hole transporting material. The content of the electron-accepting compound in the hole transporting layer or the composition for hole transporting layer is generally 0.1 mol % or more, preferably 1 mol % or more, and generally 100 mol % or less, preferably 40 mol % or less.

The following materials (i) to (x) are representative examples of the hole transporting layer materials usable in an embodiment of the invention. These may be used alone or in combination, preferably in combination of a relatively electron-donating material and a relatively electron-accepting material. In addition, one or more third components may be added, for example, an additive for promoting the charge transport between the electron-donating compound and the electron-accepting compound and for improving the film-forming properties by coating may be added.

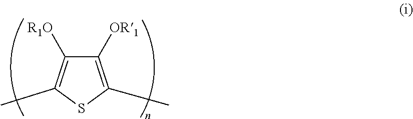

In formula (i), R₁ and R₁′ are each independently selected from a hydrogen atom and an alkyl group having 1 to 4 carbon atoms; R₁ and R₁′ may be bonded to each other to form an alkylene chain having 1 to 4 carbon atoms; the alkylene chain optionally has a substituent selected from an alkyl group having 1 to 12 carbon atoms, an aromatic group having 6 to 12 carbon atoms, and a 1,2-cyclohexylene group; and n represents a number larger than 6.

A polyaniline comprising a monomer unit represented by formula (ii) and/or (iii):

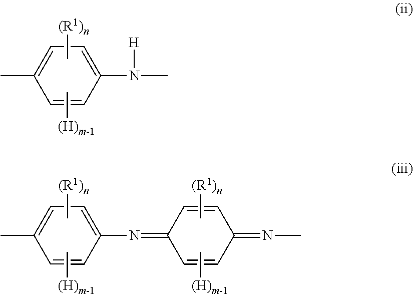

wherein n represents an integer of 0 to 4;

m−1 represents an integer of 1 to 5, and n+(m−1)=5;

each R¹ may be the same or different and is independently selected from an alkyl group, an alkenyl group, an alkoxy group, a cycloalkyl group, a cycloalkenyl group, an alkanoyl group, an alkylthio group, an aryloxy group, an alkylthioalkyl group, an alkylaryl group, an arylalkyl group, an amino group, an alkylamino group, a dialkylamino group, an aryl group, an alkylsulfinyl group, an alkoxyalkyl group, an alkylsulfonyl group, an arylthio group, an arylsulfinyl group, an alkoxycarbonyl group, an arylsulfonyl group, a carboxyl group, a halogen atom, a cyano group, an alkyl group having one or more substituents selected from a sulfonic acid group, a carboxyl group, a halogen atom, a nitro group, a cyano group, and an epoxy group; and

adjacent two group R¹ may be bonded to each other to form an alkylene chain or an alkenylene chain each completing a 3-, 4-, 5-, 6- or 7-membered aromatic or aliphatic ring which may include at least one of a nitrogen atom, a sulfur atom and an oxygen atom.

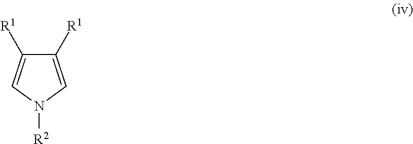

In formula (iv), each R¹ is independently selected from a hydrogen atom, an alkyl group, an alkenyl group, an alkoxy group, an alkanoyl group, an alkylthio group, an aryloxy group, an alkylthioalkyl group, an alkylaryl group, an arylalkyl group, an amino group, an alkylamino group, a dialkylamino group, an aryl group, an alkylsulfinyl group, an alkoxyalkyl group, an alkylsulfonyl group, an arylthio group, an arylsulfinyl group, an alkoxycarbonyl group, an arylsulfonyl group, an acrylic acid group, a phosphoric acid group, a phosphonic acid group, a halogen atom, a nitro group, a cyano group, a hydroxyl group, an epoxy group, a silyl group, a siloxane group, an alcohol group, a benzyl group, a carboxylate group, an ether group, an ether carboxylate group, an amide sulfonate group, an ether sulfonate group, and an urethane group;

adjacent two group R¹ may be bonded to each other to form an alkylene chain or an alkenylene chain each completing a 3-, 4-, 5-, 6- or 7-membered aromatic or aliphatic ring which may include at least one of a nitrogen atom, a sulfur atom and an oxygen atom; and

R² is selected from a hydrogen atom, an alkyl group, an alkenyl group, an aryl group, an alkanoyl group, an alkylthioalkyl group, an alkylaryl group, an arylalkyl group, an amino group, an epoxy group, a silyl group, a siloxane group, an amide sulfonate group, an alcohol group, a benzyl group, a carboxylate group, an ether group, an ether carboxylate group, an amide sulfonate group, an ether sulfonate group, and an urethane group.

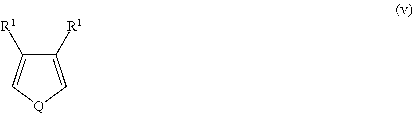

In formula (v), Q is selected from the group consisting of S, Se, and Te;

each R¹ is independently selected from a hydrogen atom, an alkyl group, an alkenyl group, an alkoxy group, an alkanoyl group, an alkylthio group, an aryloxy group, an alkylthioalkyl group, an alkylaryl group, an arylalkyl group, an amino group, an alkylamino group, a dialkylamino group, an aryl group, an alkylsulfinyl group, an alkoxyalkyl group, an alkylsulfonyl group, an arylthio group, an arylsulfinyl group, an alkoxycarbonyl group, an arylsulfonyl group, an acrylic acid group, a phosphoric acid group, a phosphonic acid group, a halogen atom, a nitro group, a cyano group, a hydroxyl group, an epoxy group, a silyl group, a siloxane group, an alcohol group, a benzyl group, a carboxylate group, an ether group, an ether carboxylate group, an amide sulfonate group, an ether sulfonate group, an ester sulfonate group, and an urethane group; and

adjacent two group R¹ may be bonded to each other to form an alkylene chain or an alkenylene chain each completing a 3-, 4-, 5-, 6- or 7-membered aromatic or aliphatic ring which may include at least one of a nitrogen atom, a selenium atom, a tellurium atom, a sulfur atom, and an oxygen atom.

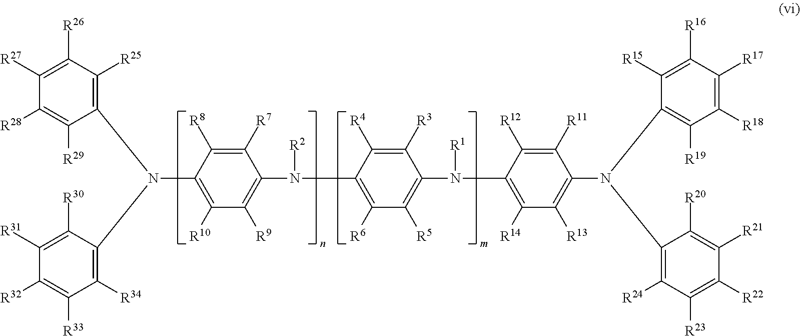

In formula (vi), R¹ and R² each independently represent a hydrogen atom, a substituted or unsubstituted monovalent hydrocarbon group, a t butoxycarbonyl group, or a benzyloxycarbonyl group;

R³ to R³⁴ each independently represent a hydrogen atom, a hydroxyl group, a silanol group, a thiol group, a carboxyl group, a phosphoric acid group, a phosphoric ester group, an ester group, a thioester group, an amido group, a nitro group, a substituted or unsubstituted monovalent hydrocarbon group, an organooxy group, an organoamino group, an organosilyl group, an organothio group, an acyl group, a sulfone group, or a halogen atom; and

m and n each independently represent an integer of 1 or more, which satisfy m+n≦20.

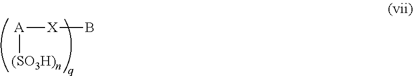

In formula (vii), X represents O, S or NH;

A represents a naphthalene ring or an anthracene ring which may have a substituent other than X and (SO₃H)n groups;

B represents a substituted or unsubstituted hydrocarbon group, a 1,3,5-triazine group, or a group represented by formula (vii-1) or (vii-2) which may have a substituent:

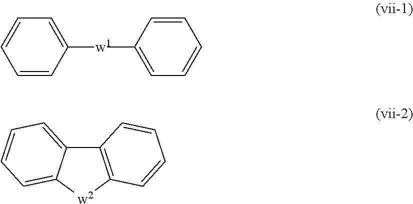

wherein W¹ and W² each independently represent O, S, S(O), or S(O₂), or represent N, Si, P, or P(O) which may have a substituent; and

n represents an integer satisfying 1≦n≦4 and q is an integer satisfying 1≦q.

In view of improving the durability and increasing the charge transporting ability, B is preferably a substituted or unsubstituted di- or more valent hydrocarbon group comprising at least one aromatic ring, a di- or tri-valent 1,3,5-triazine group, or a substituted or unsubstituted di-valent diphenylsulfone group, and particularly preferably a substituted or unsubstituted di- or tri-valent benzyl group, a substituted or unsubstituted divalent p-xylylene group, a substituted or unsubstituted di- or tri-valent naphthyl group, a di- or tri-valent 1,3,5-triazine group, a substituted or unsubstituted divalent diphenylsulfone group, a di- to tetra-valent perfluorobiphenyl group, a substituted or unsubstituted divalent 2,2-bis((hydroxypropoxy)phenyl)propyl group, or a substituted or unsubstituted polyvinylbenzyl group.

The compound represented by formula (vii) is particularly preferably represented by formula (vii-3):

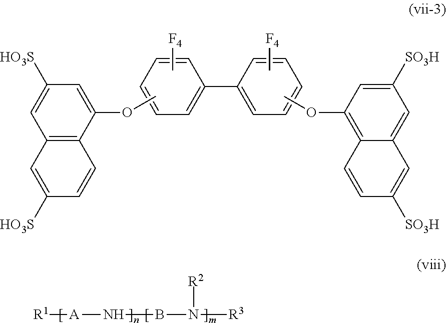

In formula (viii), R¹, R², and R³ each independently represent a hydrogen atom, a halogen atom, a hydroxyl group, an amino group, a silanol group, a thiol group, a carboxyl group, a phosphoric acid group, a phosphoric ester group, an ester group, a thioester group, an amido group, a nitro group, a monovalent hydrocarbon group, an organooxy group, an organoamino group, an organosilyl group, an organothio group, an acyl group, or a sulfonic acid group; and

A and B each independently represent a divalent group represented by formula (viii-1) or (viii-2):

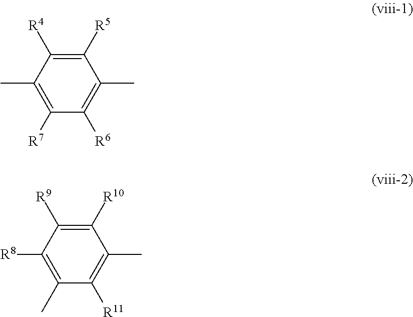

wherein R⁴ to R¹¹ each independently represent a hydrogen atom, a halogen atom, a hydroxyl group, an amino group, a silanol group, a thiol group, a carboxyl group, a phosphoric acid group, a phosphoric ester group, an ester group, a thioester group, an amido group, a nitro group, a monovalent hydrocarbon group, an organooxy group, an organoamino group, an organosilyl group, an organothio group, an acyl group, or a sulfonic acid group; and

m and n each independently represent an integer of 1 or more satisfying m+n≦20.

The material (ix) is a mixture of the following compounds:

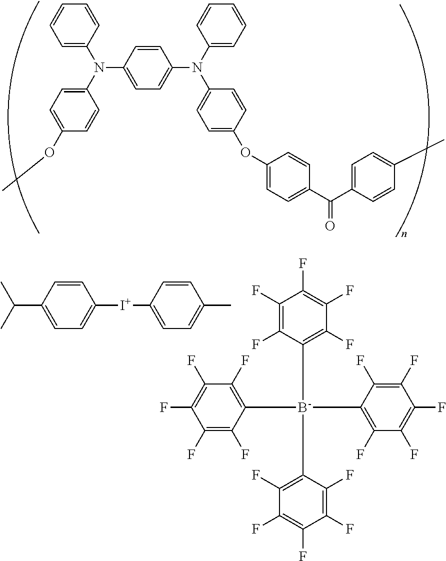

wherein n is an integer of 3 or more.

The material (X) is a mixture of the following compounds:

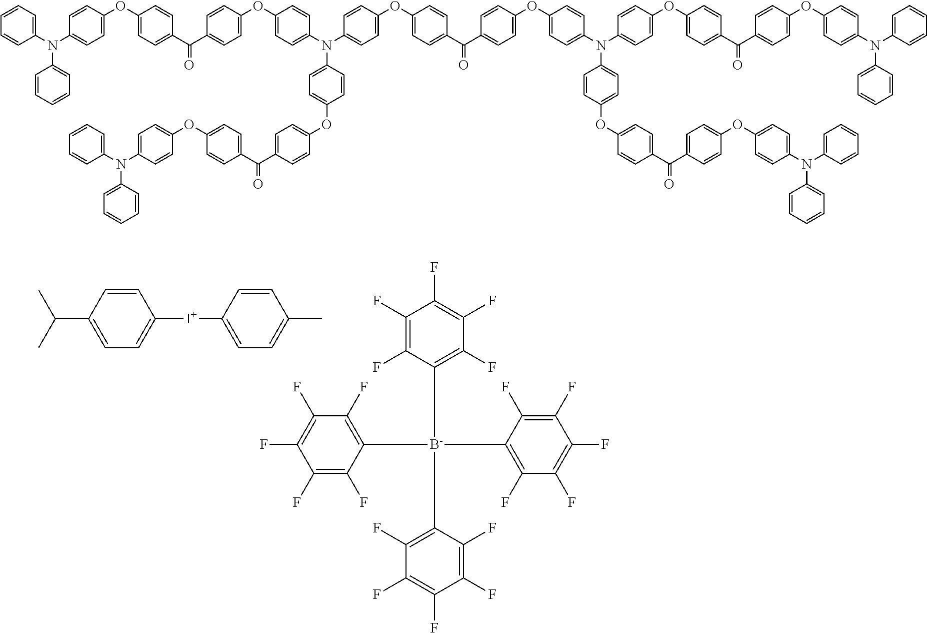 [SMILES: O=C(c1ccc(Oc2ccc(N(c3ccccc3)c3ccccc3)cc2)cc1)c1ccc(Oc2ccc(N(c3ccc(Oc4ccc(C(=O)c5ccc(Oc6ccc(N(c7ccccc7)c7ccccc7)cc6)cc5)cc4)cc3)c3ccc(Oc4ccc(C(=O)c5ccc(Oc6ccc(N(c7ccc(Oc8ccc(C(=O)c9ccc(Oc%10ccc(N(c%11ccccc%11)c%11ccccc%11)cc%10)cc9)cc8)cc7)c7ccc(Oc8ccc(C(=O)c9ccc(Oc%10ccc(N(c%11ccccc%11)c%11ccccc%11)cc%10)cc9)cc8)cc7)cc6)cc5)cc4)cc3)cc2)cc1 ; Cc1ccc([I+]c2ccc(C(C)C)cc2)cc1 ; Fc1c(F)c(F)c([B-](c2c(F)c(F)c(F)c(F)c2F)(c2c(F)c(F)c(F)c(F)c2F)c2c(F)c(F)c(F)c(F)c2F)c(F)c1F]

In an embodiment of the invention, a phenylamine-based polymer represented by formula (x) is also usable as the hole transporting material:

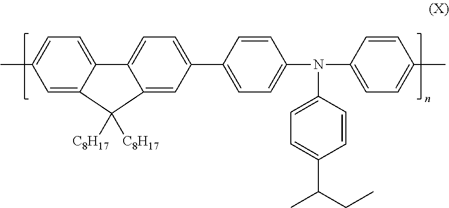

wherein n is an integer of 3 or more.

In an embodiment of the invention, the hole transporting layer may be made into a two-layered structure of a first hole transporting layer (anode side) and a second hole transporting layer (cathode side).

The thickness of the hole transporting layer is preferably 10 to 200 nm, although not particularly limited thereto.

In an embodiment of the invention, a layer comprising an acceptor material may be formed in contact with the anode side of the hole transporting layer or the first hole transporting layer. With such a layer, it is expected that the driving voltage is lowered and the production cost is reduced.

The acceptor material is preferably a compound represented by formula (Y):

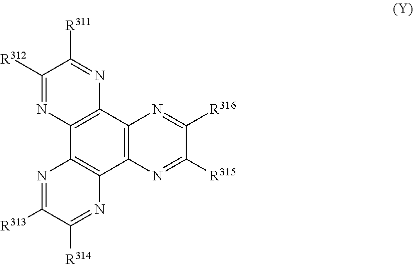

wherein R³¹¹ to R³¹⁶ may be the same or different and each independently represent a cyano group, —CONH₂, a carboxyl group, or —COOR³¹⁷ wherein R³¹⁷ represents an alkyl group having 1 to 20 carbon atoms or a cycloalkyl group having 3 to 20 carbon atoms; and one or more pairs selected from R³¹¹ and R³¹², R³¹³ and R³¹⁴, and R³¹⁵ and R³⁶ may bond to each other to form a group represented by —CO—O—CO—.

Examples of R³¹⁷ include a methyl group, an ethyl group, a n-propyl group, an isopropyl group, a n-butyl group, an isobutyl group, a t-butyl group, a cyclopentyl group, and a cyclohexyl group.

The thickness of the layer comprising the acceptor material is preferably 5 to 20 nm, although not particularly limited thereto.

The following compounds may be used as the acceptor material.

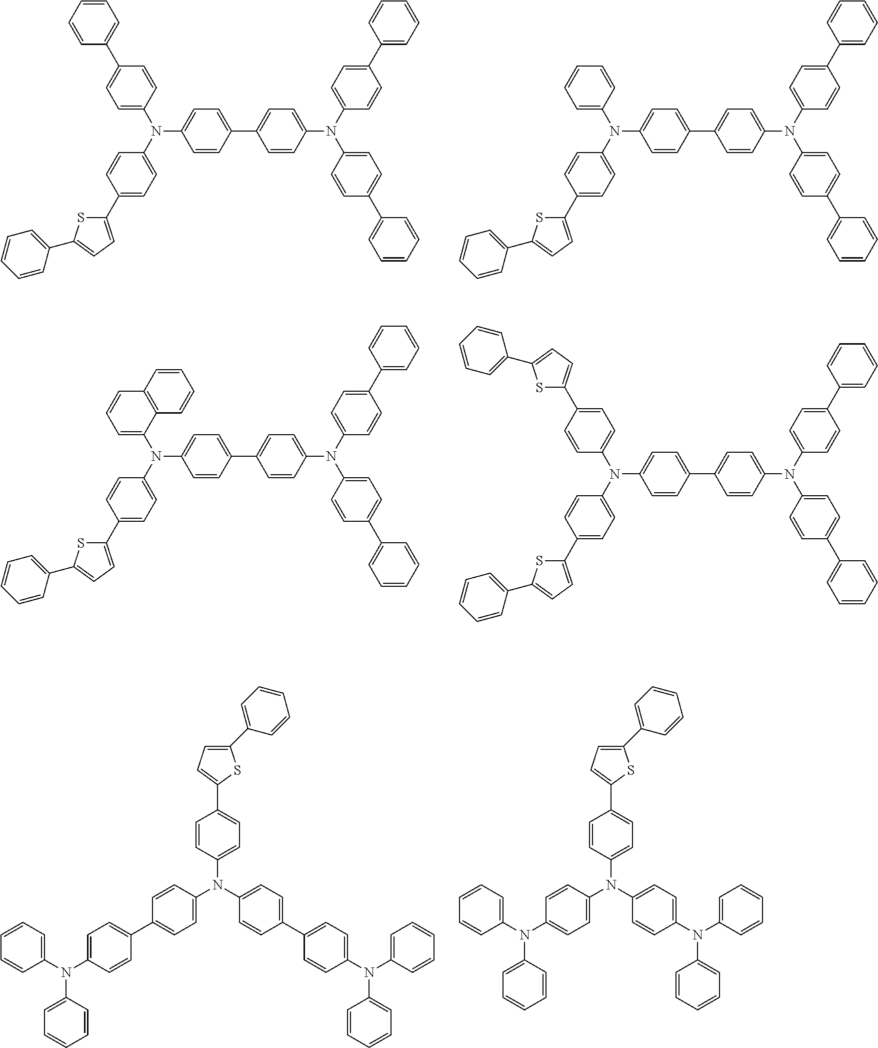

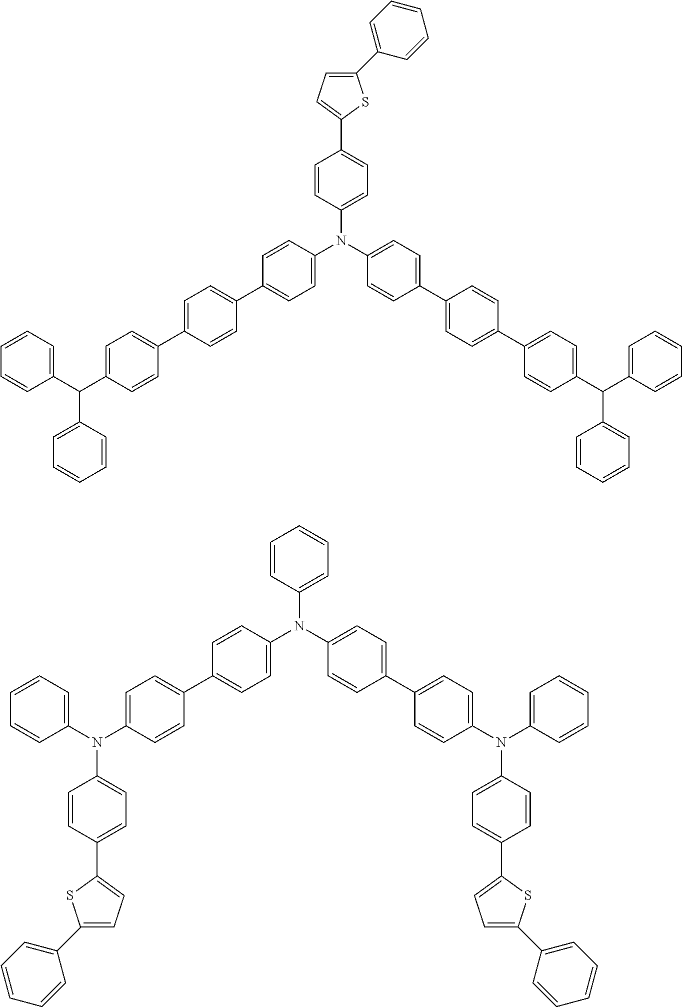 [SMILES: c1ccc(-c2ccc(-c3ccc(N(c4ccc(-c5ccc(-c6ccc(C(c7ccccc7)c7ccccc7)cc6)cc5)cc4)c4ccc(-c5ccc(-c6ccc(C(c7ccccc7)c7ccccc7)cc6)cc5)cc4)cc3)s2)cc1 ; c1ccc(-c2ccc(-c3ccc(N(c4ccccc4)c4ccc(-c5ccc(N(c6ccccc6)c6ccc(-c7ccc(N(c8ccccc8)c8ccc(-c9ccc(-c%10ccccc%10)s9)cc8)cc7)cc6)cc5)cc4)cc3)s2)cc1]

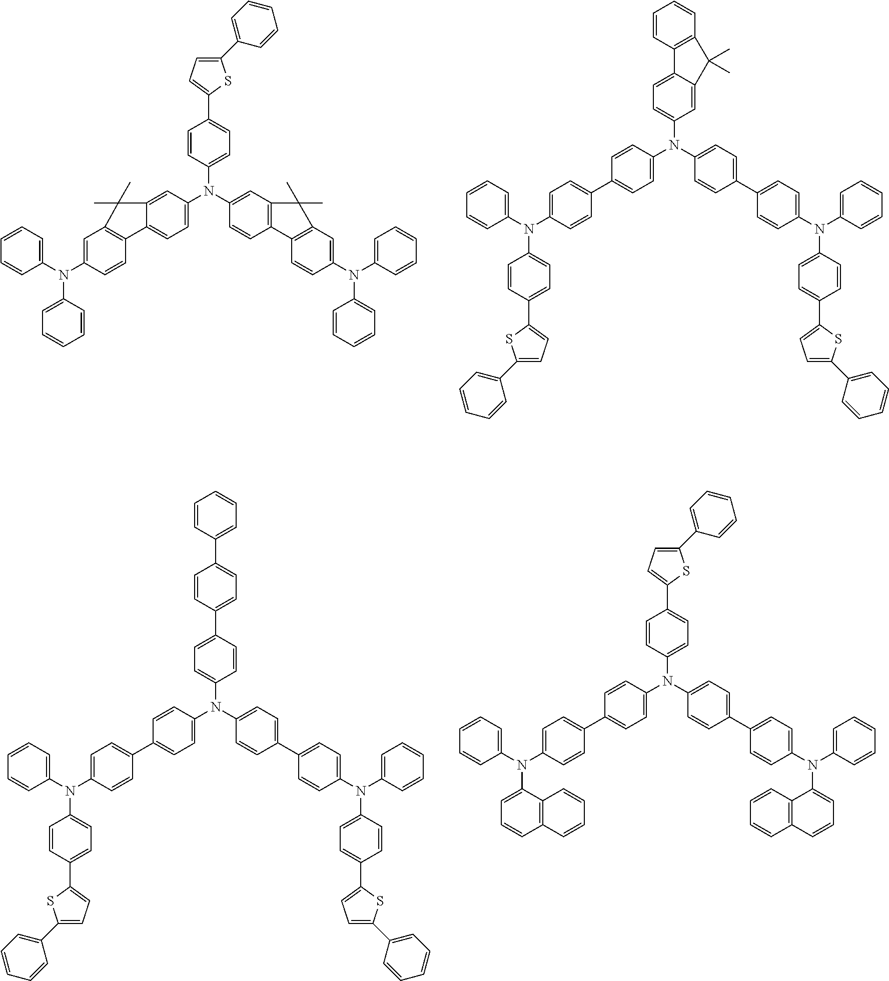

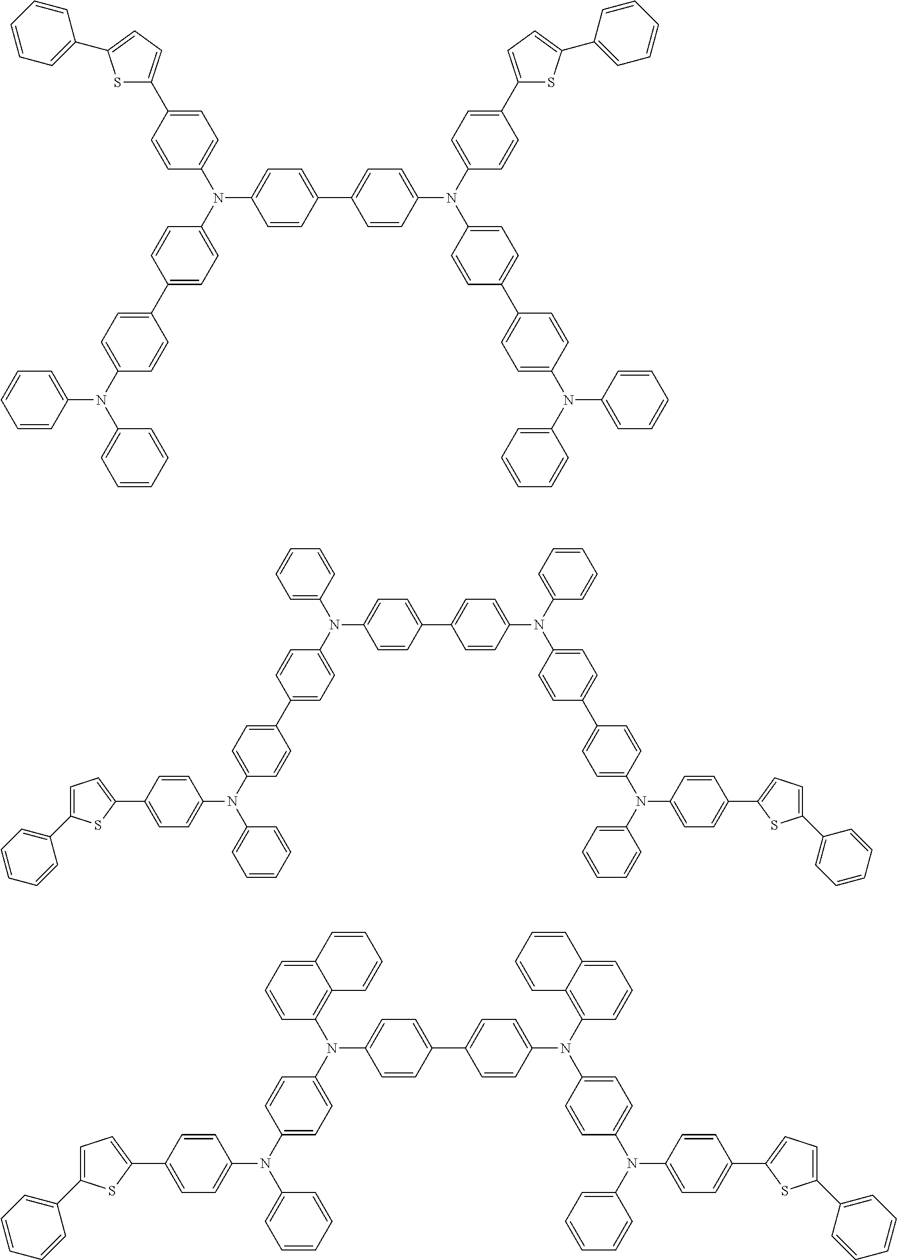

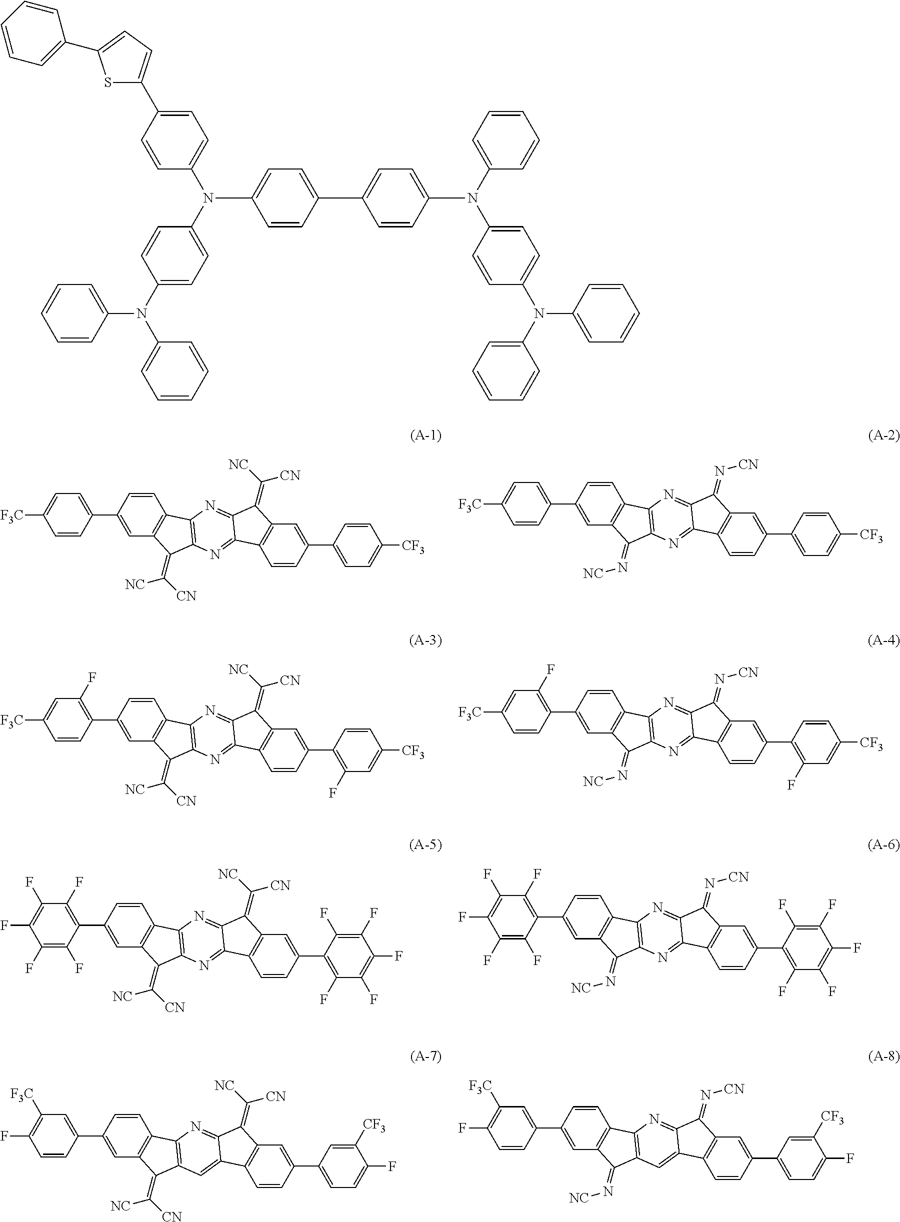

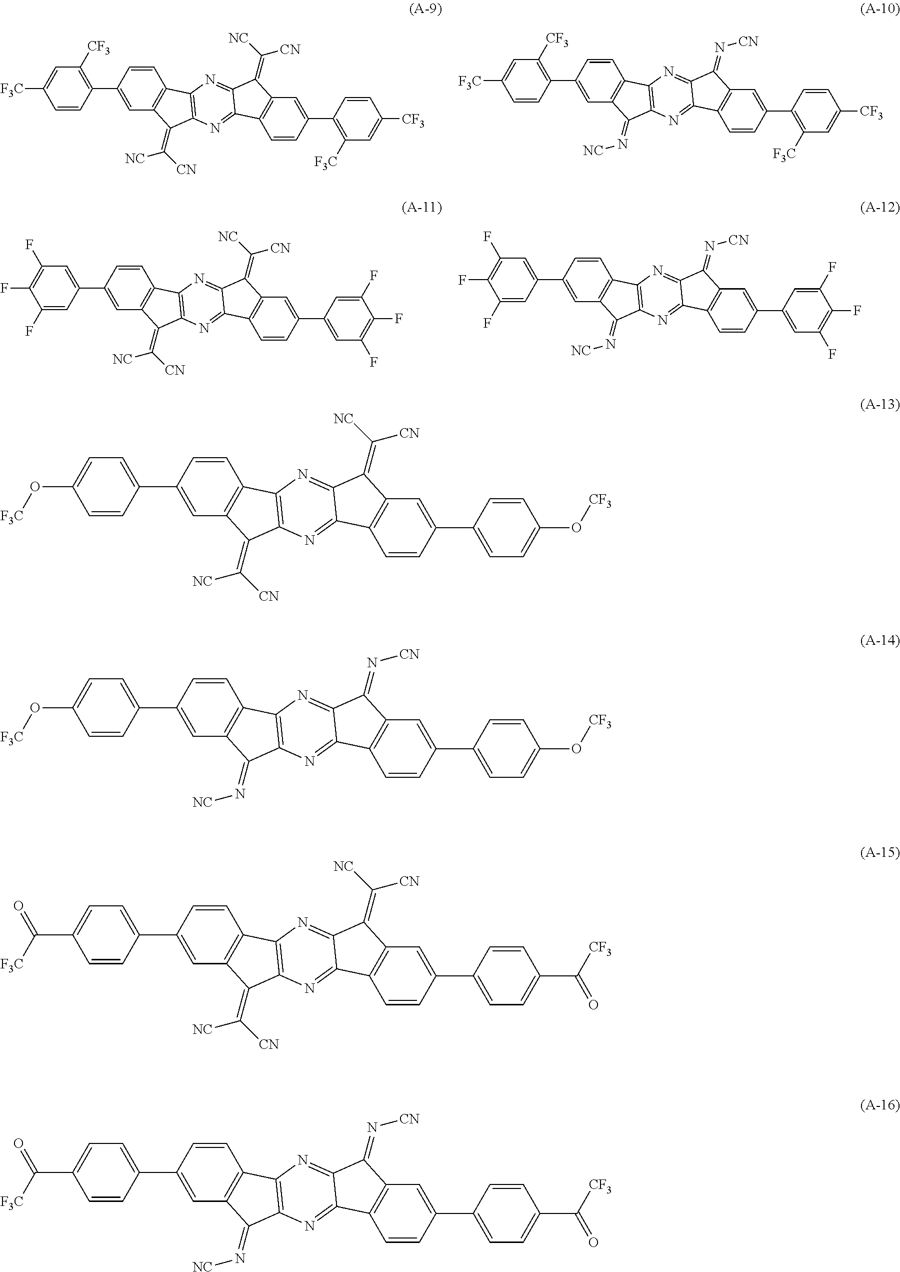

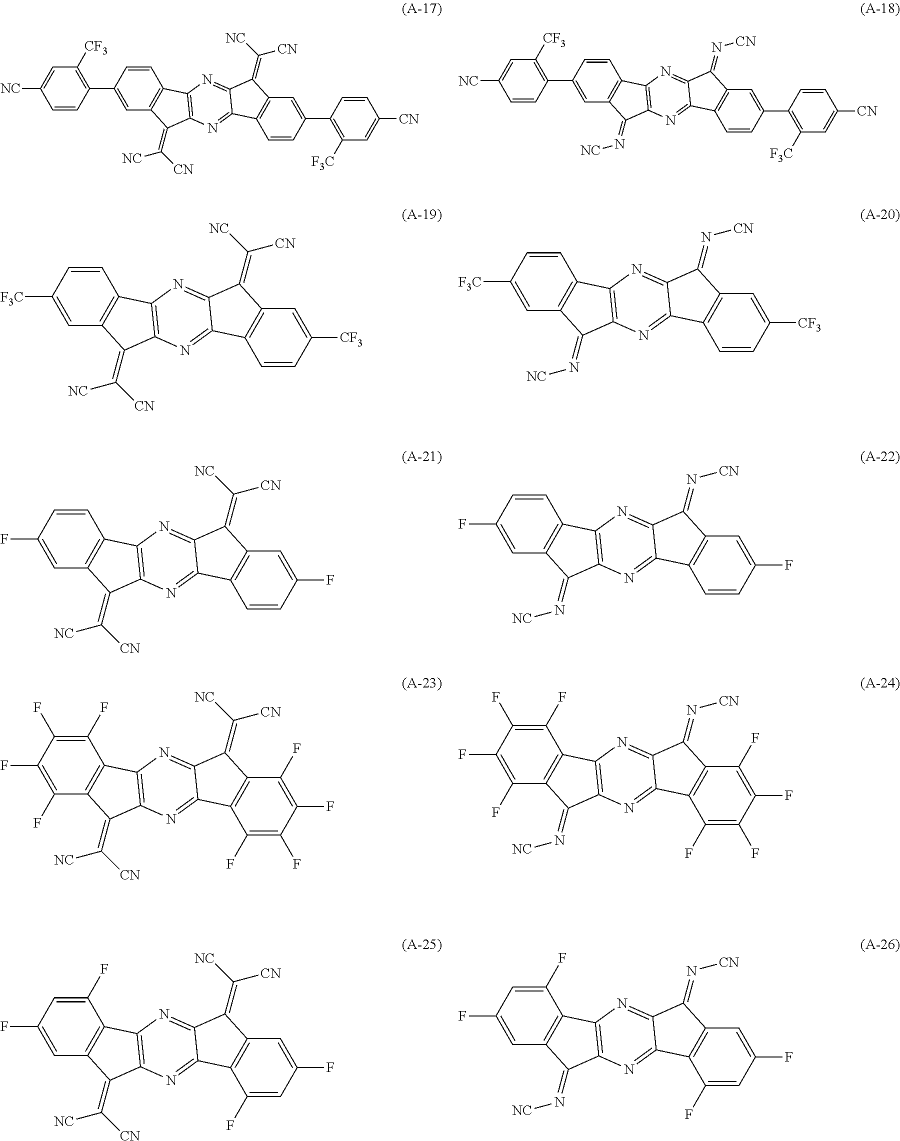

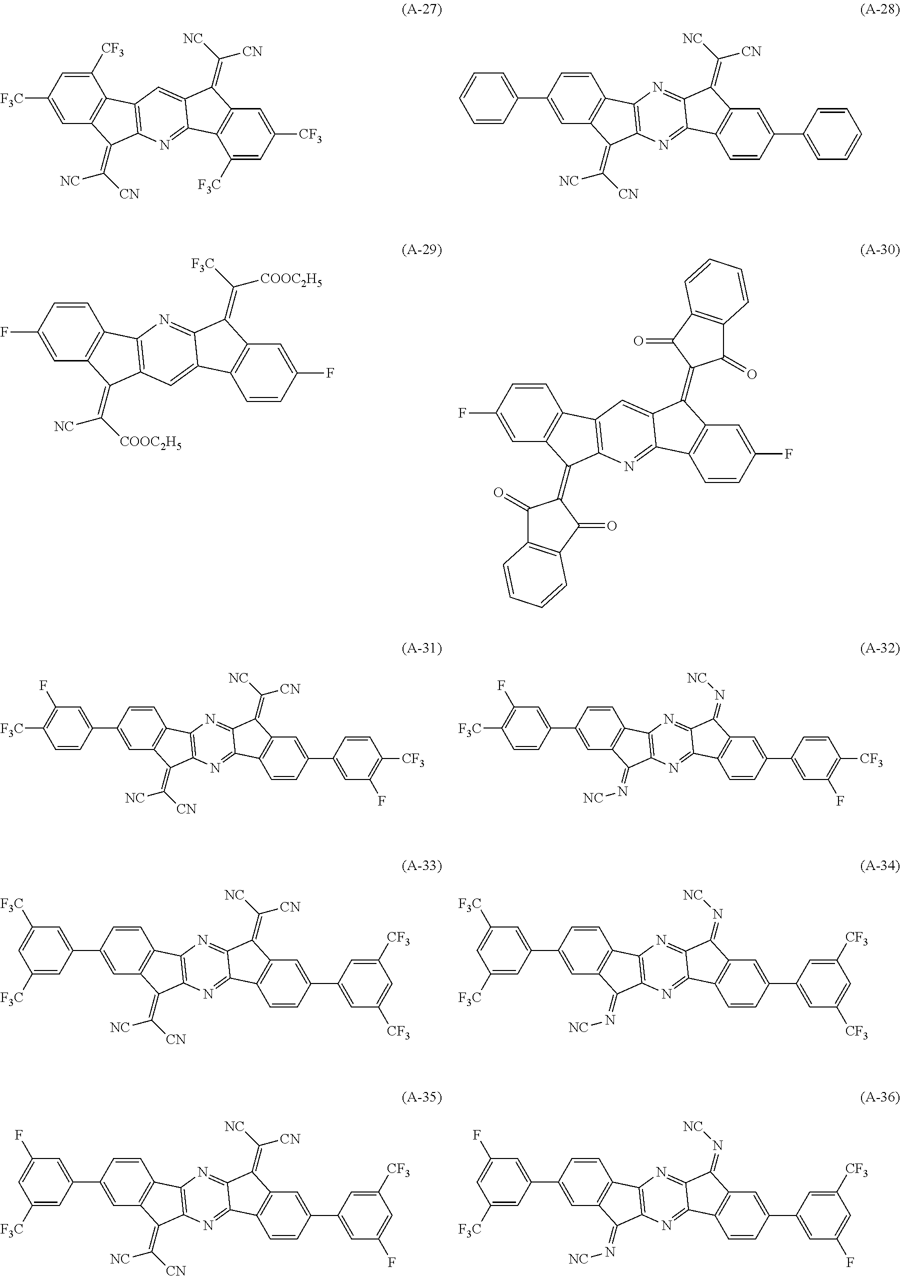

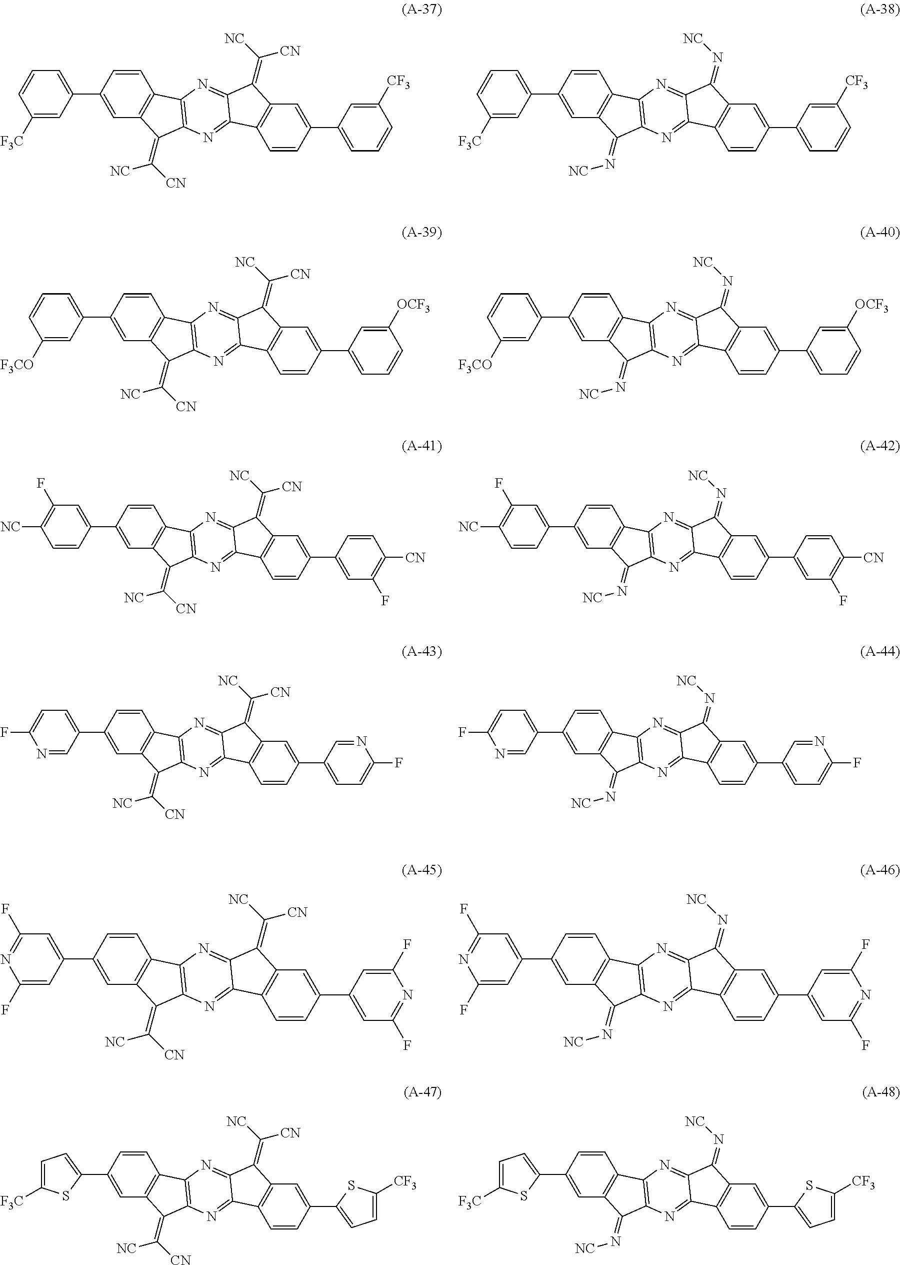

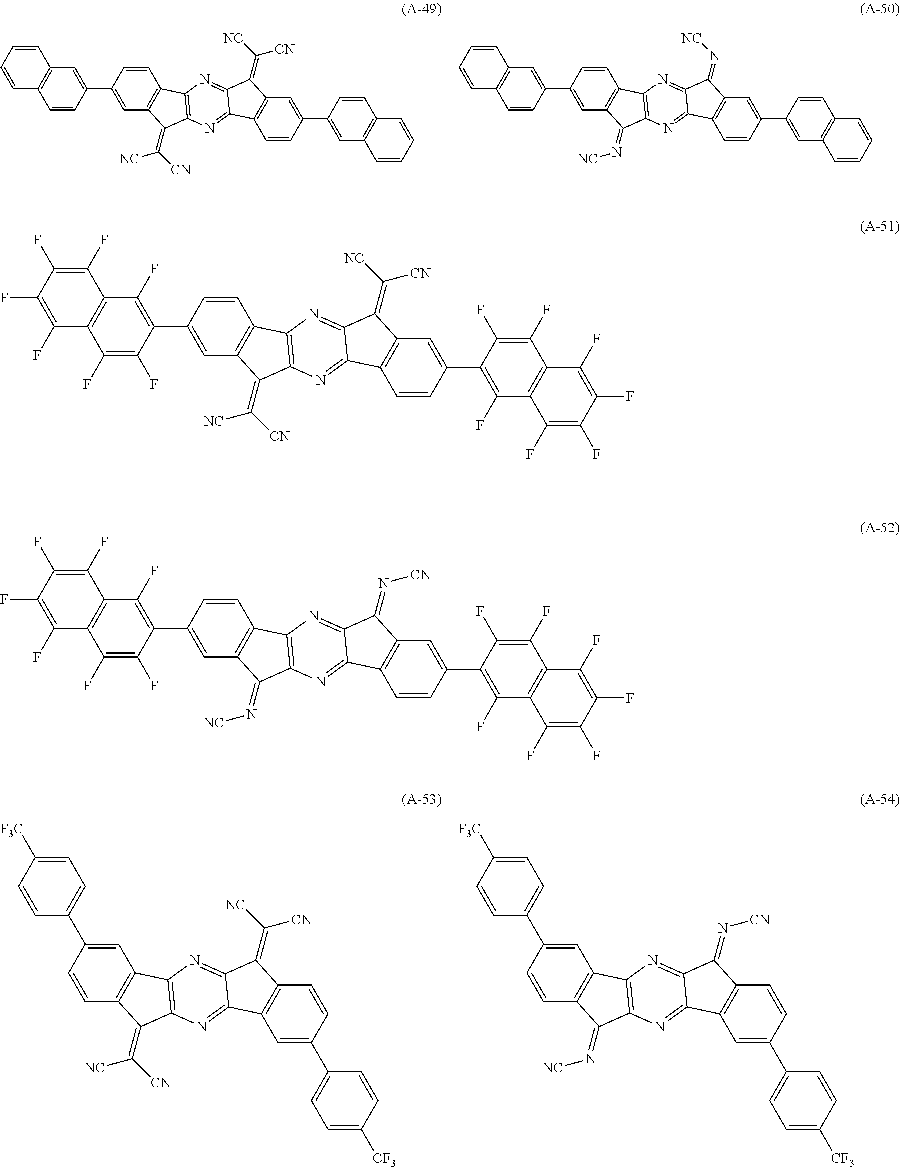

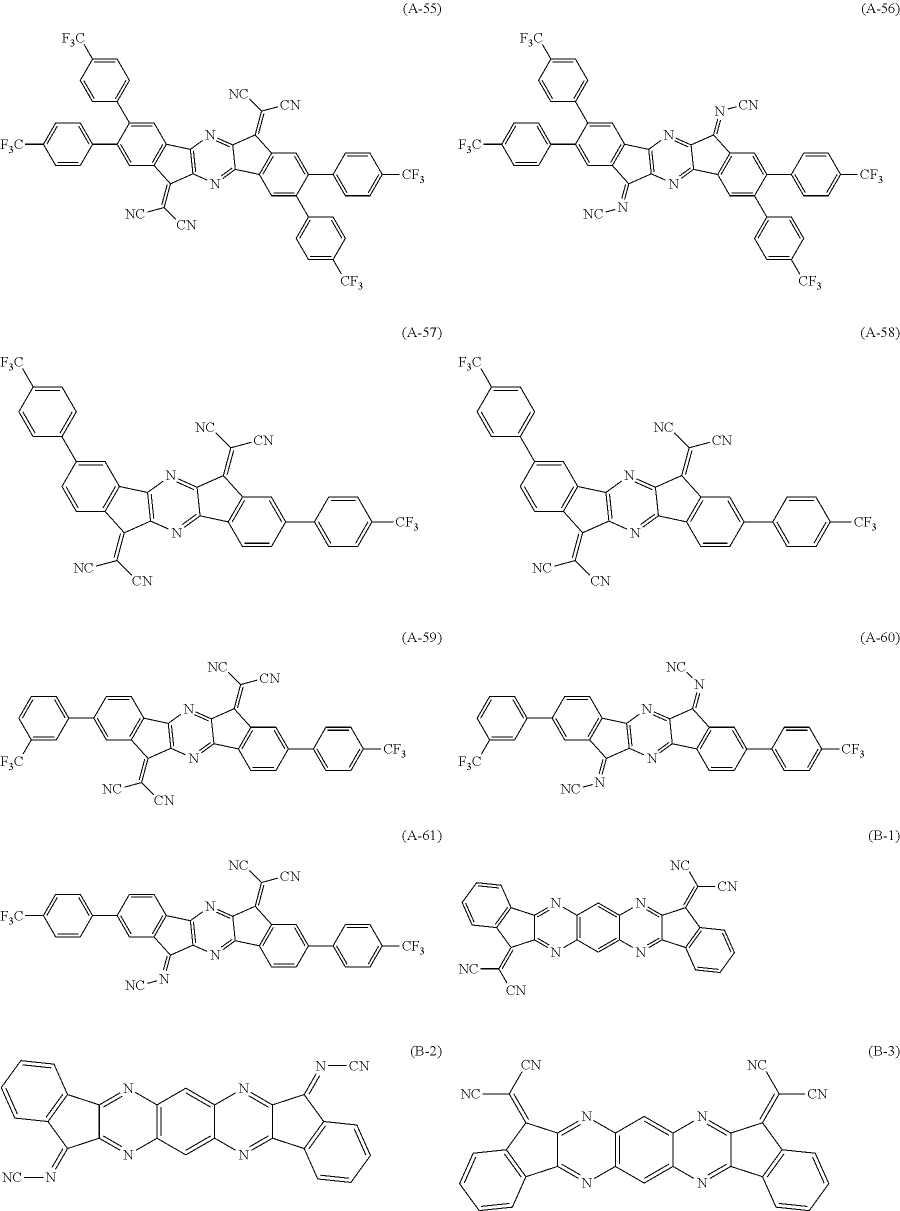

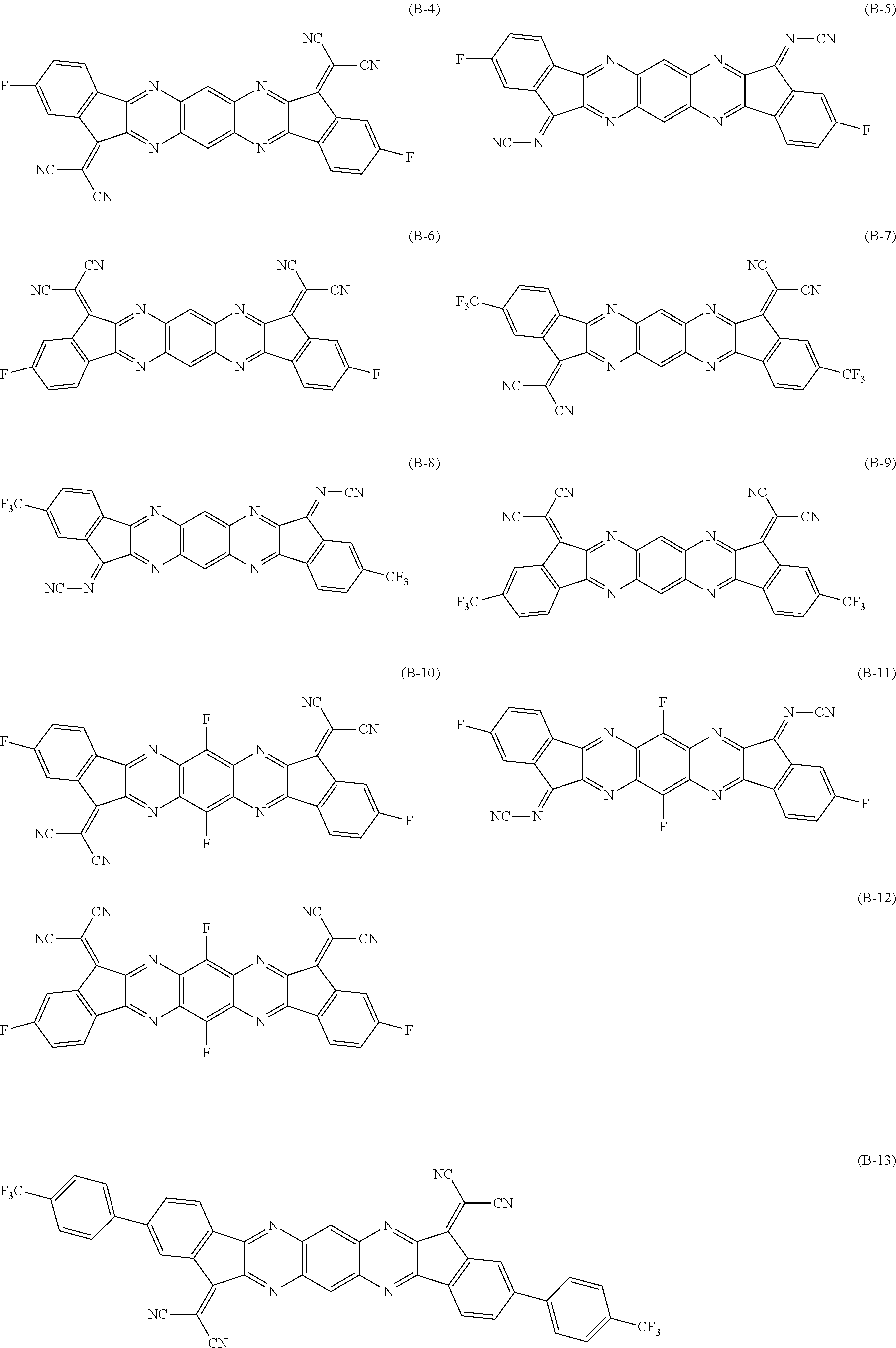

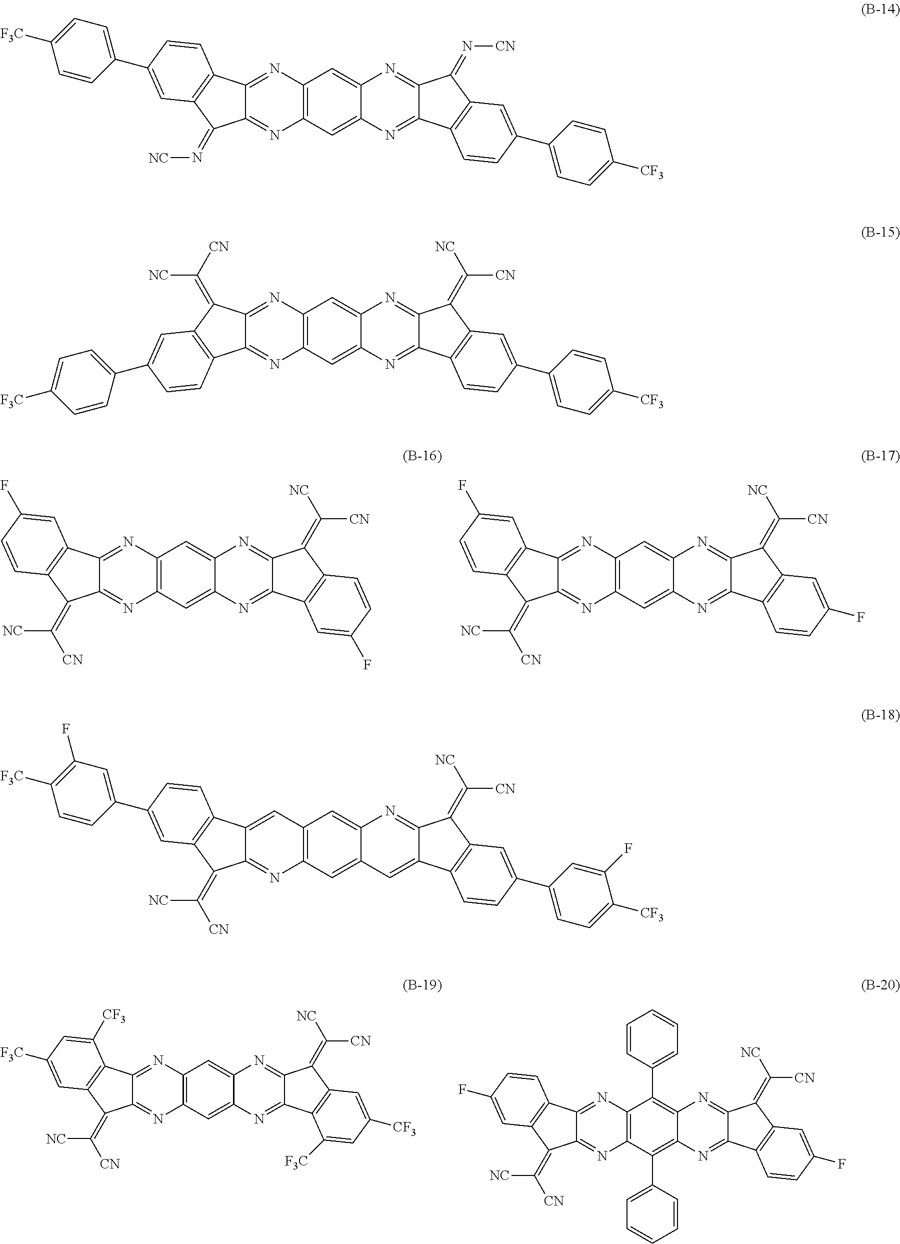

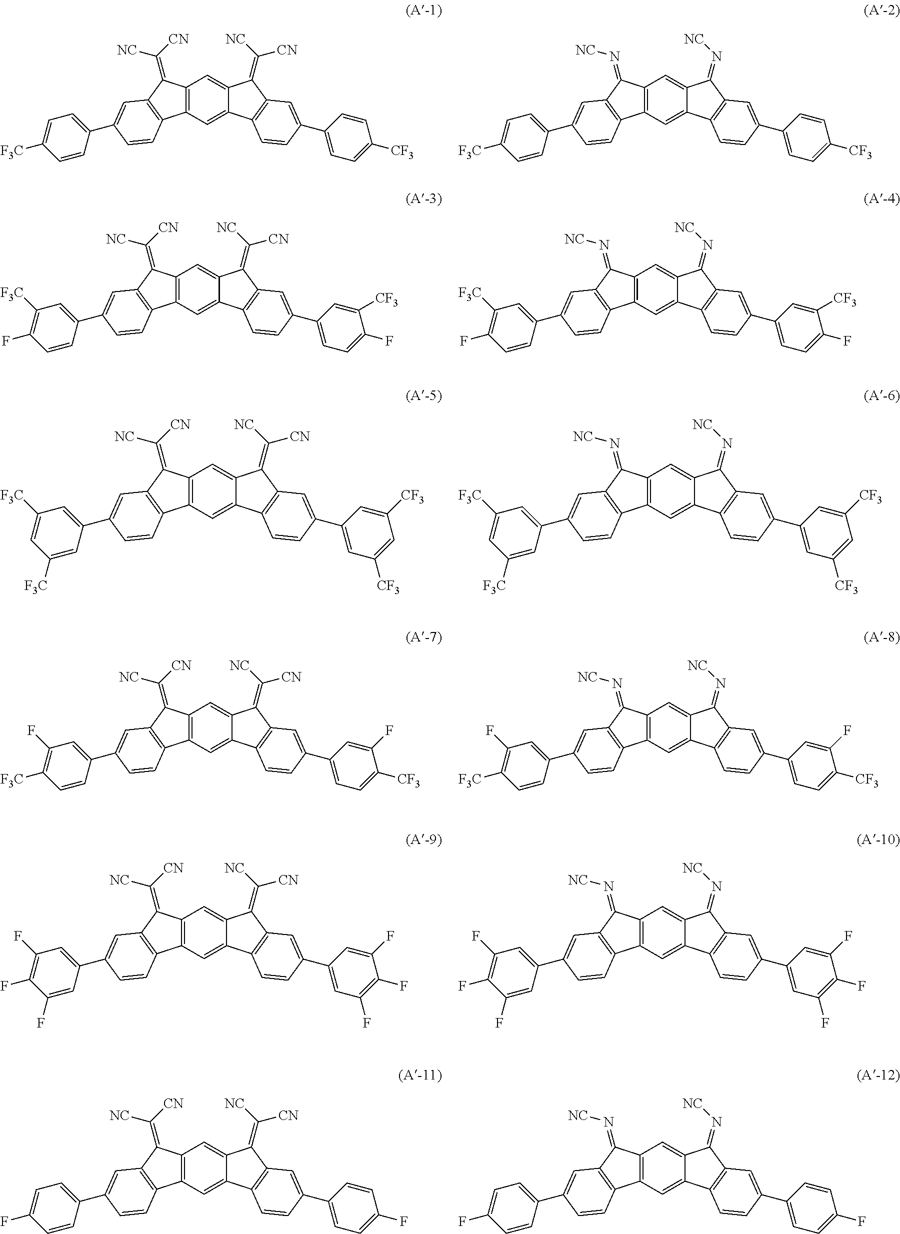

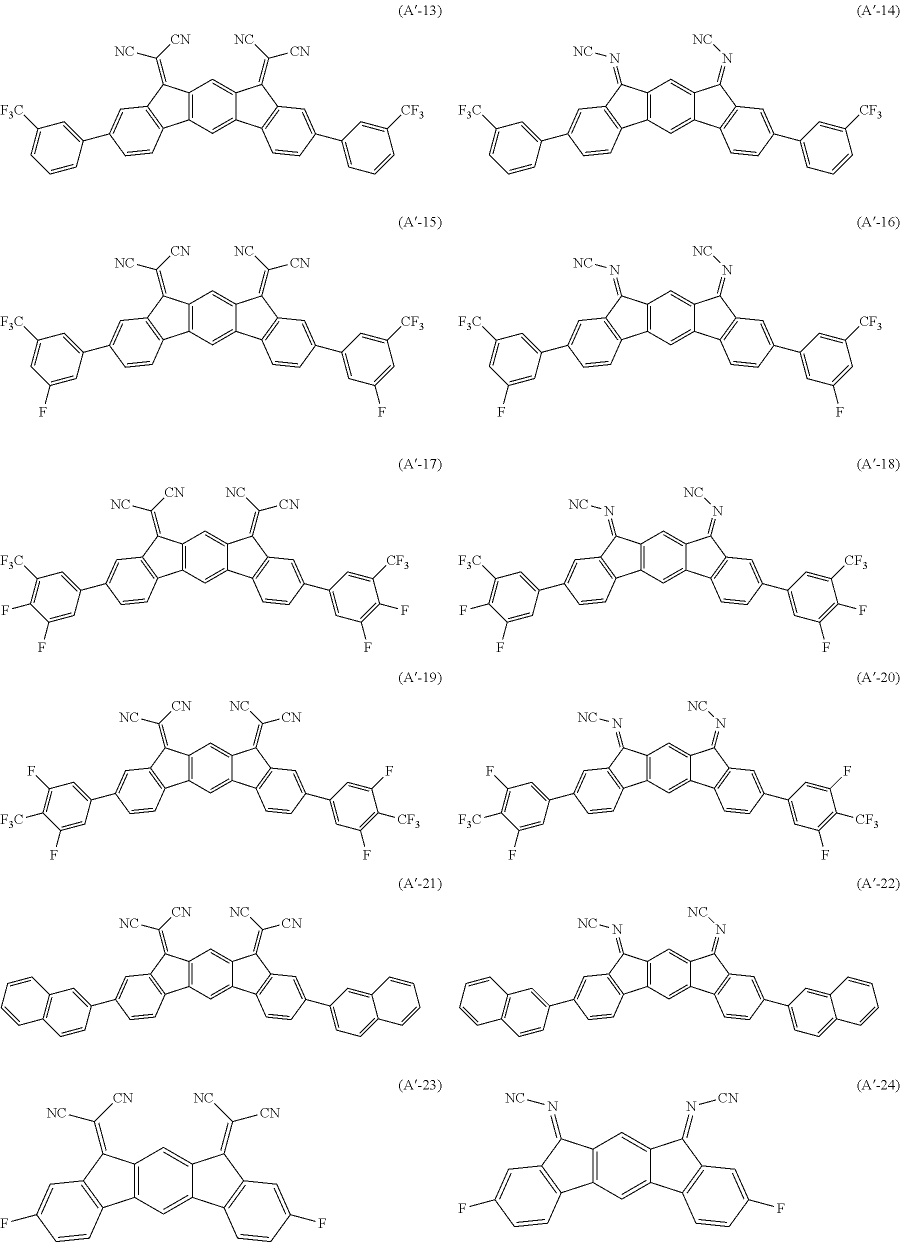

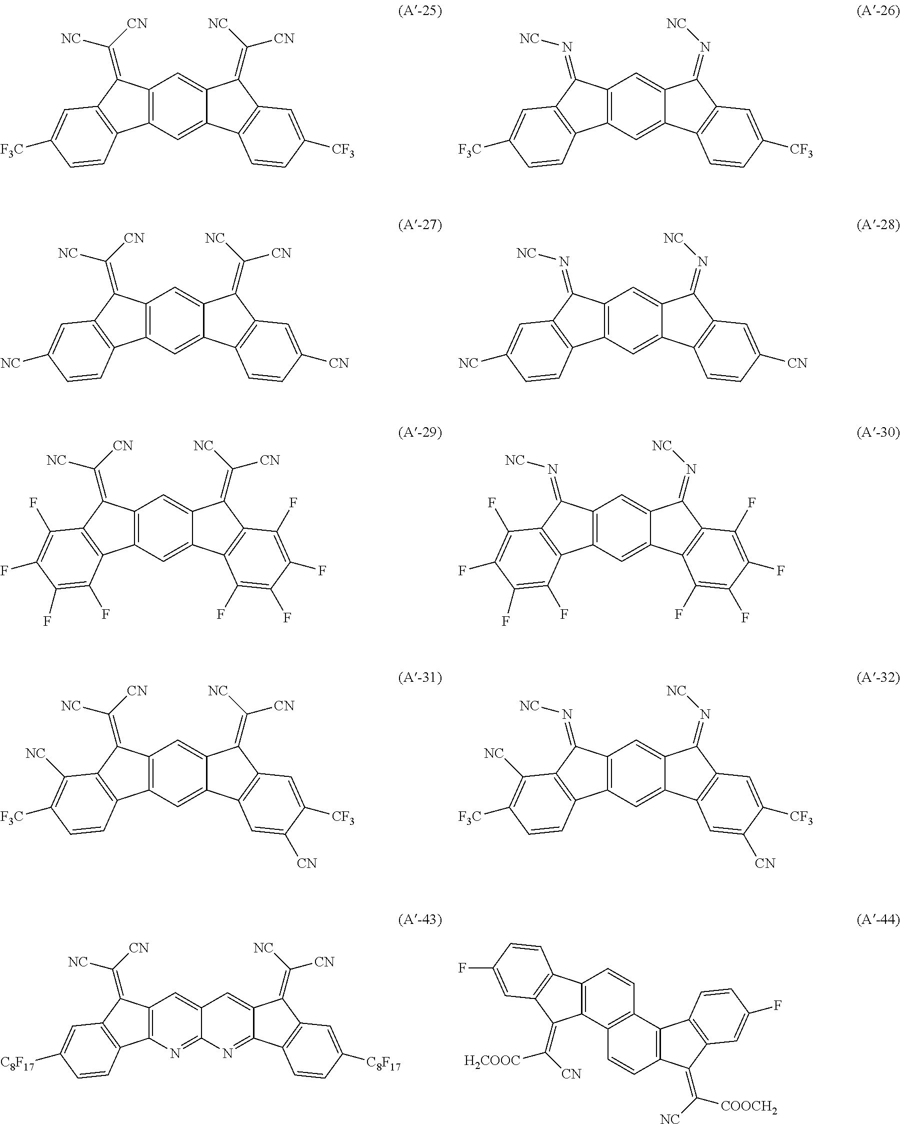

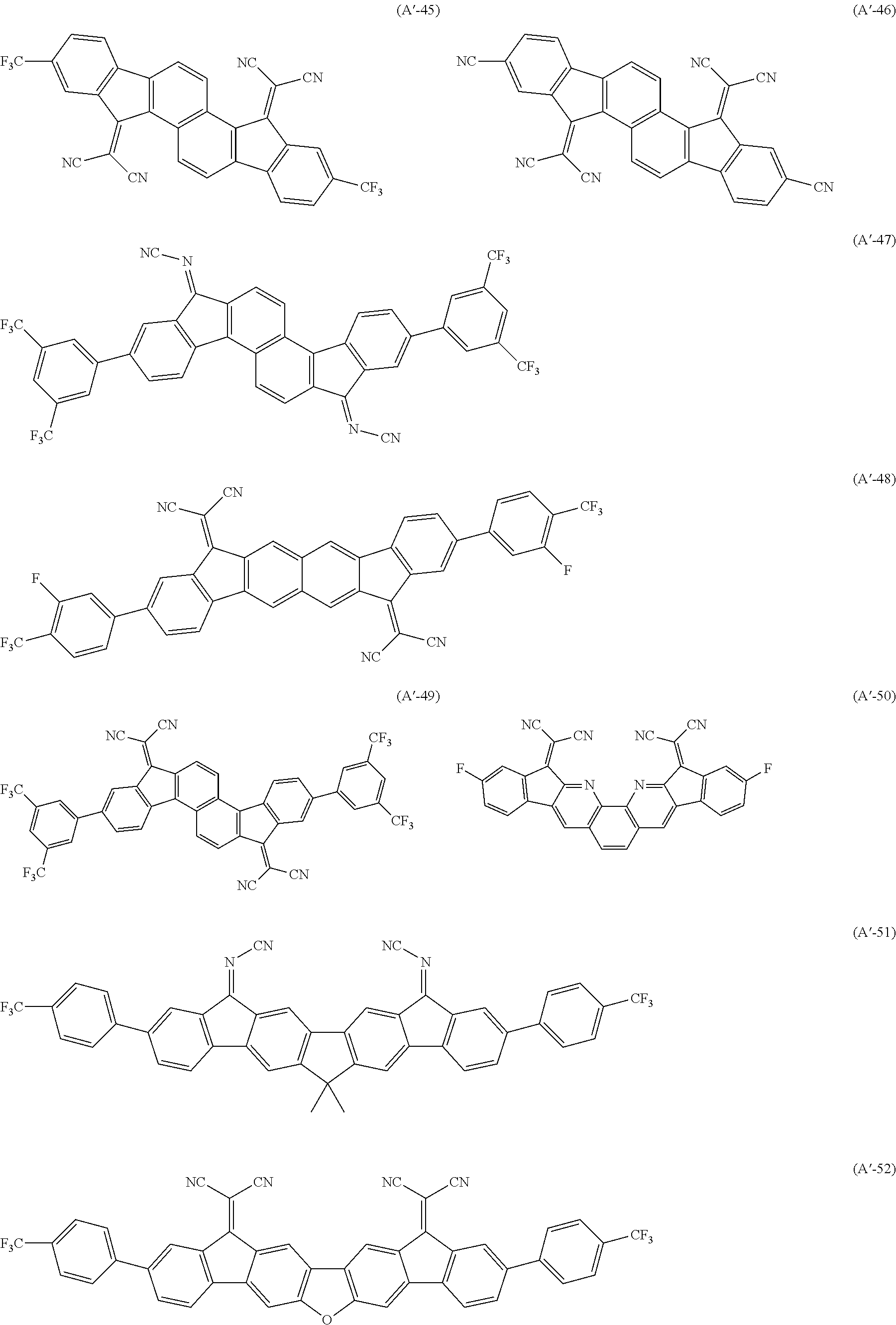

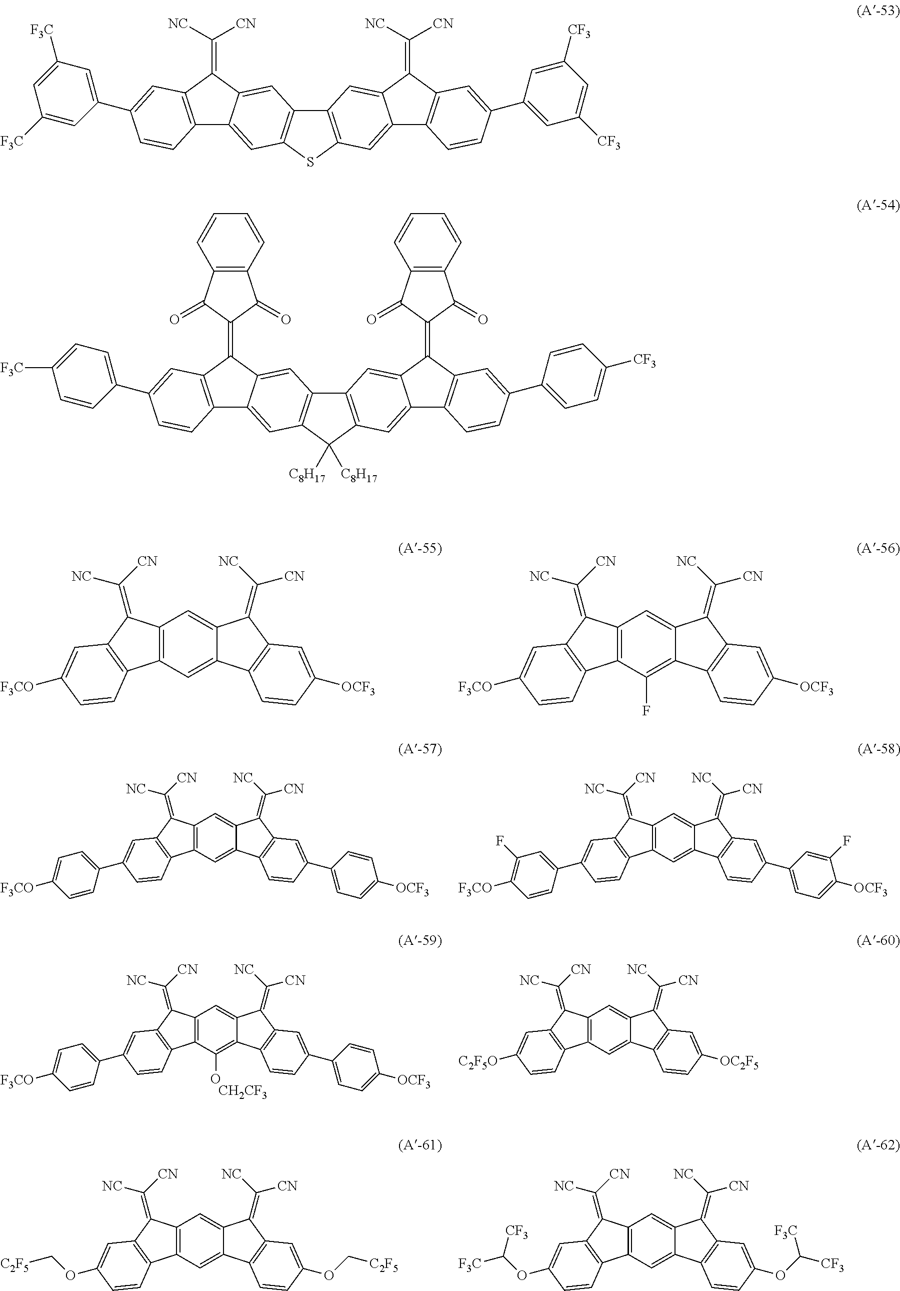

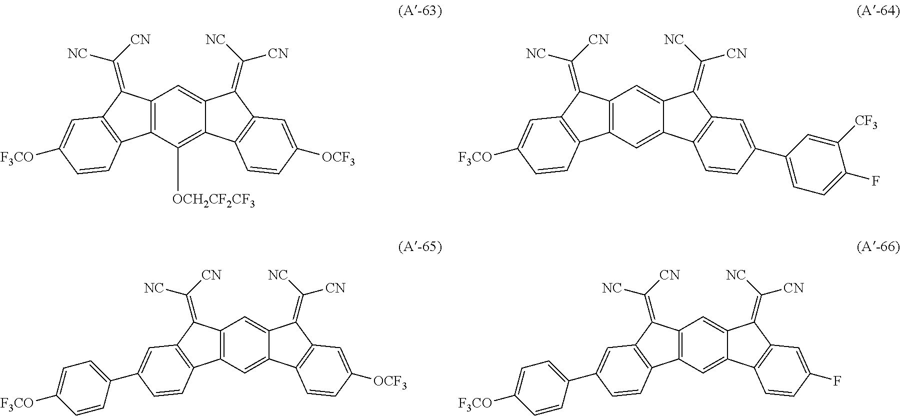

### N/P Doping

The carrier injecting properties of the hole transporting layer and the electron transporting layer can be controlled by the doping (n) with a donor material or the doping (p) with an acceptor material.

A typical example of the n-doping is an electron transporting material doped with a metal, such as Li and Cs, and a typical example of the p-doping is a hole transporting material doped with an acceptor material, such as F₄TCNQ (2,3,5,6-Tetrafluoro-7, 7,8,8-tetracyanoquinodimethane).

### Space Layer

The space layer is a layer, for example, disposed between a fluorescent emitting layer and a phosphorescent emitting layer to prevent the diffusion of excitons generated in the phosphorescent emitting layer to the fluorescent emitting layer or to control the carrier balance. The space layer may be disposed between two or more phosphorescent emitting layers.

Since the space layer is disposed between the light emitting layers, a material combining the electron transporting ability and the hole transporting ability is preferably used as a material for the space layer. To prevent the diffusion of triplet energy in the adjacent phosphorescent emitting layer, the triplet energy of the material for the space layer is preferably 2.6 eV or more. The materials described above with respect to the hole transporting layer are usable as the material for the space layer. The material for organic EL devices in an aspect of the invention may be used as the material for the space layer.

### Blocking Layer

In an embodiment of the invention, a blocking layer, such as an electron blocking layer, a hole blocking layer, and a triplet blocking layer, is preferably formed adjacent to the light emitting layer. The electron blocking layer is a layer which prevents the diffusion of electrons from the light emitting layer to the hole transporting layer. The material for organic EL devices in an aspect of the invention may be used as the material for hole blocking layer.

The triplet blocking layer prevents the diffusion of triplet excitons generated in a light emitting layer to adjacent layers and has a function of confining the triplet excitons within a light emitting layer, thereby preventing the deactivation of energy on a molecule other than the emitting dopant of triplet excitons, for example, on a molecule in an electron transporting layer.

If a phosphorescent device having a triplet blocking layer satisfies the following energy relationship:

E_{T}^{d}<E^{T}_{TB}

wherein E^{T}_{d} is the triplet energy of a phosphorescent dopant in a light emitting layer and E^{T}_{TB} is the triplet energy of a compound forming the triplet blocking layer, the triplet excitons of phosphorescent dopant are energetically confined (not move into other molecules). Therefore, the energy deactivation process other than the emission on the phosphorescent dopant may be prevented, thereby enabling the emission with high efficiency. However, even in case of satisfying the relationship of E^{T}_{d}<E^{T}_{TB}, the triplet excitons may move into other molecules if the energy difference (ΔE^{T}=E^{T}_{TB}−E^{T}_{d}) is small, because the energy difference ΔE^{T} may be overcome by the absorption of the ambient heat energy when a device is operated at around room temperature as generally employed in practical operation. As compared with the fluorescent emission, the phosphorescent emission is likely to be affected by the endothermic diffusion of excitons because the lifetime of triplet excitons is longer. Therefore, as for the energy difference ΔE^{T}, the larger as compared with the heat energy of room temperature, the better, i.e., the energy difference ΔE^{T} is more preferably 0.1 eV or more and particularly preferably 0.2 eV or more. In a fluorescent device, the material for organic EL devices in an aspect of the invention may be used as the material for triplet blocking layer.

The electron mobility of the material for the triplet blocking layer is preferably 10⁻⁶ cm²/Vs or more at an electric field strength of 0.04 to 0.5 MV/cm. There are several methods for measuring the electron mobility of organic material, for example, Time of Flight method. In the present invention, the electron mobility is determined by an impedance spectroscopy.

The electron mobility of the electron injecting layer is preferably 10⁻⁶ cm²/Vs or more at an electric field strength of 0.04 to 0.5 MV/cm. Within the above range, the injection of electrons from the cathode to the electron transporting layer is promoted and the injection of electrons to the adjacent blocking layer and the light emitting layer is also promoted, thereby enabling to drive a device at lower voltage.

The method of forming each layer of the organic EL device in an aspect of the invention is not particularly limited, and each layer of the organic EL device may be formed by any of known methods, such as a vacuum vapor deposition method and a spin coating method. The organic thin film layer comprising the compound (1) may be formed by a known method, for example, a vacuum vapor deposition method, a molecular beam epitaxy method (MBE method) or a coating method using a solution of the compound (1) in a solvent, such as a dipping method, a spin coating method, a casting method, a bar coating method and a roll coating method.

The thickness of each organic layer of the organic EL device is not particularly limited and preferably several nanometers to 1 μm because an excessively small thickness may cause defects, such as pin holes, and an excessively large thickness may require a high applied voltage to reduce the efficiency. The layer, particularly a light emitting layer, comprising the compound (1) of the invention is preferably formed, for example, by coating the ink composition in an aspect of the invention mentioned above.

The layer (a light emitting layer, a hole transporting layer, an electron transporting layer, etc.) comprising the compound (1) of the invention is preferably formed by the coating method mentioned above while using a solution (ink composition) comprising a solvent and the compound mentioned above. The ink composition may comprise another material such as a dopant, if needed.

The coating method is preferably a wet film-forming method, for example, a letterpress printing method, an intaglio printing method, a lithographic printing method, a stencil printing method, a combination of the preceding methods with an offset printing method, an inkjet printing method, a dispenser coating method, a spin coating method, a bar coating method, a dip coating method, a spray coating method, a slit coating method, a roll coating method, a cap coating method, a rotogravure roll coating method, and a meniscus coating method. If a fine patterning is required, a letterpress printing method, an intaglio printing method, a lithographic printing method, a stencil printing method, a combination of the preceding methods with an offset printing method, an inkjet printing method, and a dispenser coating method are preferred. A transfer coating method is also usable, in which the polymer is preformed into a film on a substrate by the wet film-foaming method described above and then the preformed film is transferred onto a substrate having an electrode printed thereon by a laser light or hot press. The film formation by the above methods can be made under the conditions well known to a person skilled in the art

After coating, the solvent is removed by heating (250° C. or below) and drying under vacuum, and the irradiation of light and the high temperature heating exceeding 250° C. for polymerization reaction are not needed. Therefore, the deterioration of the device in its performance due to the irradiation of light and the high temperature heating exceeding 250° C. can be prevented.

### Electronic Device

The electronic device in an aspect of the invention will be described below.

The electronic device comprises the organic electroluminescence device in an aspect of the invention. The organic electroluminescence device is usable in the electronic device, for example, as display parts, such as organic EL panel module; display devices of television sets, mobile phones, personal computer, etc.; and light emitting sources of lighting equipment and vehicle lighting equipment.

### EXAMPLES

The present invention will be described in more detail with reference to the examples. However, it should be noted that the scope of the present invention is not limited thereto.

### Synthesis Example 1 (Synthesis of Compound H-1)

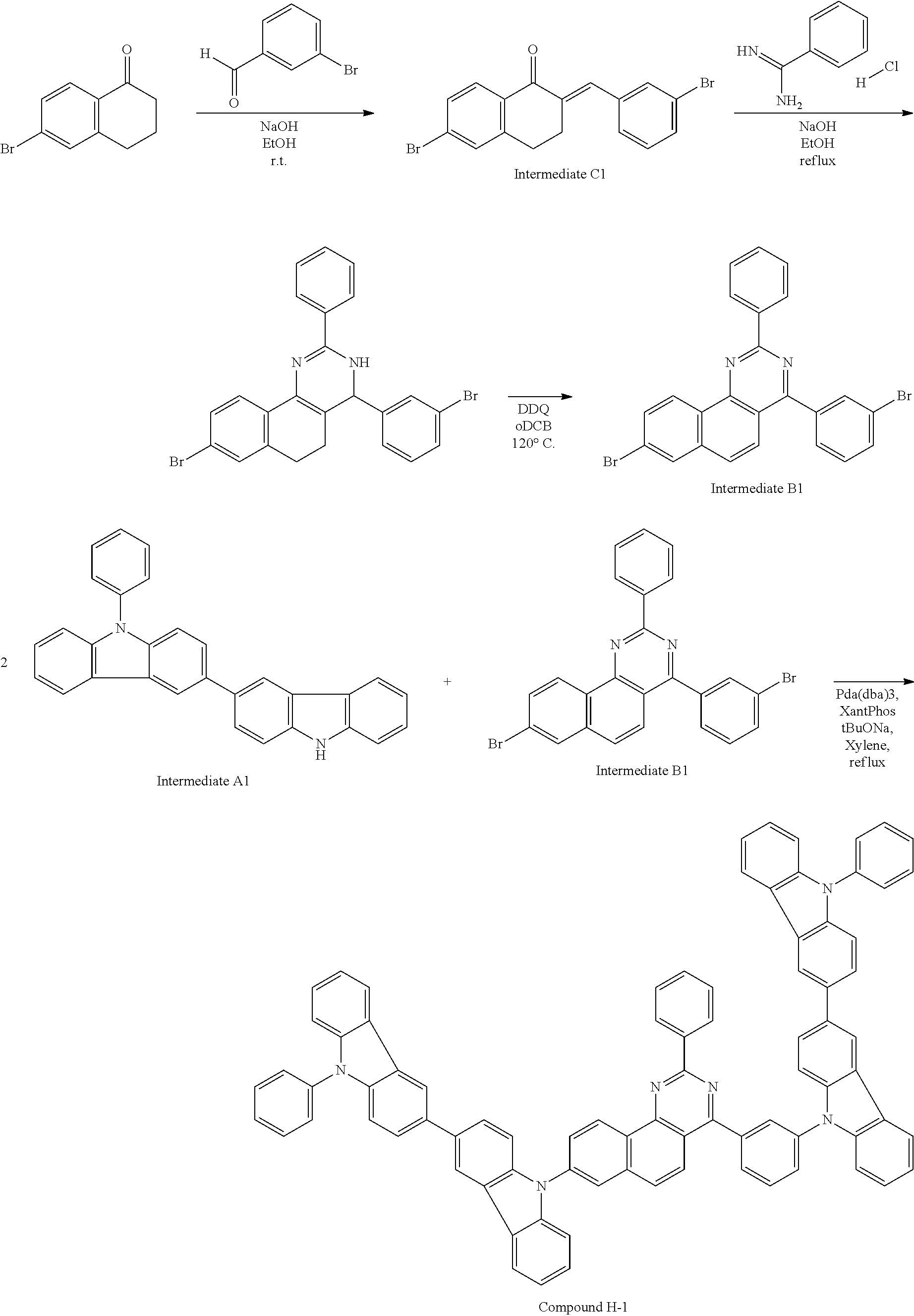

After dissolving 6-bromo-1-tetralone (11.25 g, 50 mmol) and 3-bromobenzaldehye (9.25 g, 50 mmol) in ethanol (100 mL), sodium hydroxide (0.20 g, 5 mmol) was added. The resultant mixture was stirred at room temperature for 8 h. The powder formed was collected by filtration, washed with methanol until the washings were colorless, and vacuum-dried to obtain a chalcone intermediate C1 (12.84 g, 82% yield).

The chalcone intermediate C1 (6.26 g, 20 mmol), benzamidine hydrochloride (3.13 g, 20 mmol), and sodium hydroxide (0.88 g, 22 mmol) were allowed to react in ethanol (100 mL) for 8 h by refluxing under heating. The powder formed was collected by filtration, washed with methanol, and vacuum-dried. The dried powder was allowed to react with 2,3-dichloro-5,6-dicyano-p-benzoquinone (DDQ) (9.08 g, 40 mmol) in o-dichlorobenzene (100 mL) for one hour at room temperature and then for 5 h at 120° C. After cooling to room temperature, the reaction solution was purified by silica gel column chromatography to obtain the benzoquinazoline intermediate B1 (7.01 g, 71% yield).

Under argon atmosphere, the bicarbazolyl intermediate A1 (2.57 g, 6.3 mmol), the benzoquinazoline intermediate B1 (2.94 g, 6.0 mmol), tris(dibenzylideneacetone) dipalladium (Pd₂(dba)₃) (55 mg, 0.06 mmol), 4,5′-bis(diphenylphosphino)-9,9′-dimethylxanthene (XantPhos) (69 mg, 0.12 mmol), sodium t-butoxide (0.86 g, 9.0 mmol), and anhydrous xylene (60 mL) were successively mixed, and the resultant mixture was refluxed for 12 h under heating. After cooling to room temperature, the reaction solution was filtered to remove insolubles and then the organic solvent was evaporated off under reduced pressure. The obtained residue was purified by silica gel column chromatography to obtain the compound H-1 (2.85 g, 83% yield).

The results of HPLC (High Performance Liquid Chromatography) and FD-MS analysis of the compound H-1 are shown below.

HPLC: 99.77% Purity

FD-MS: calcd for C₈₄H₅₂N₆=1144.

found m/z=1144 (M⁺, 100).

### Synthesis Example 2 (Synthesis of Compound H-2)

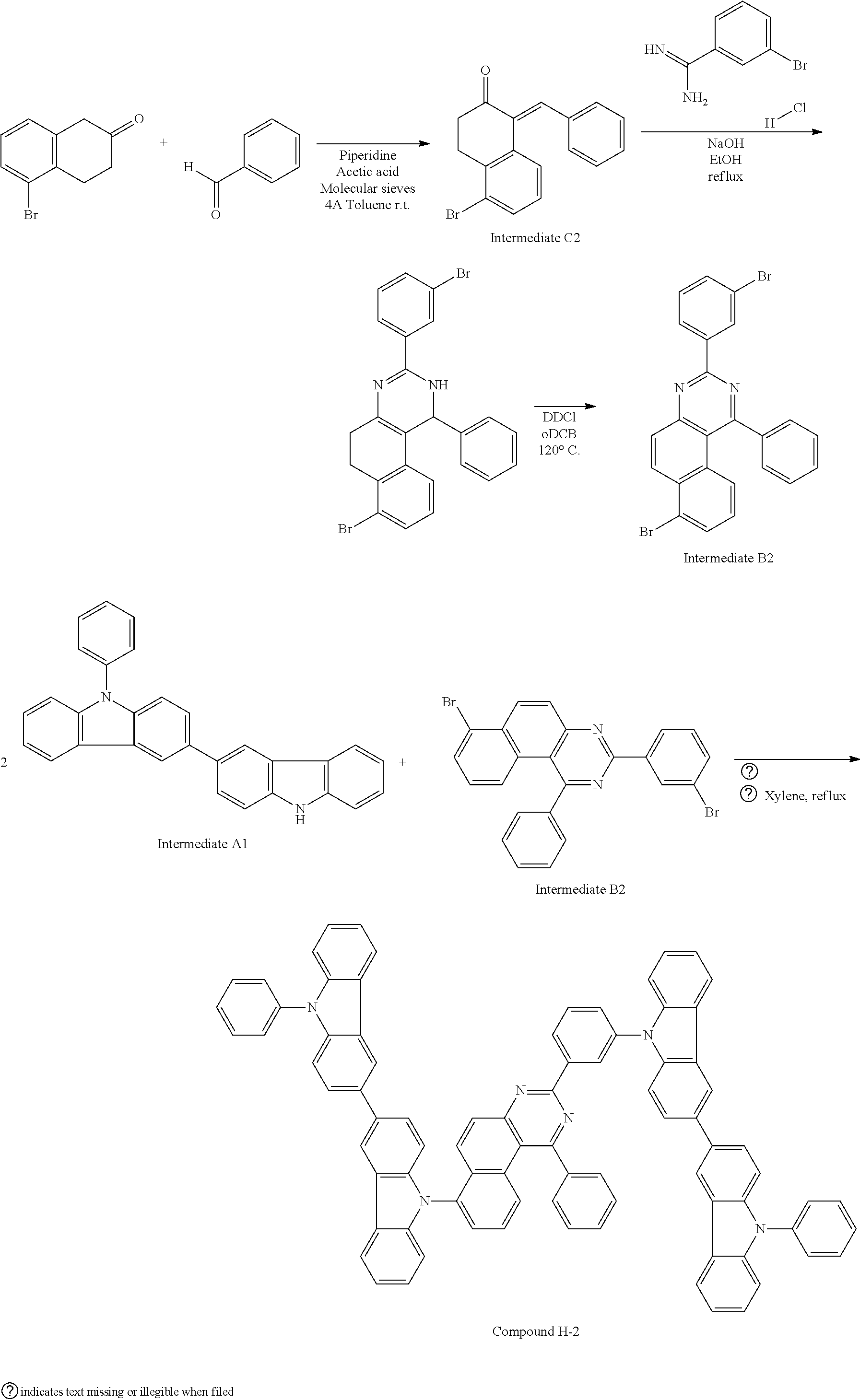

After dissolving 5-bromo-2-tetralone (11.25 g, 50 mmol) and benzaldehye (5.31 g, 50 mmol) in toluene (150 mL), piperidine (250 mg), acetic acid (250 mg), and Molecular Sieves 4A (12.5 g) were added. The resultant mixture was stirred at room temperature for 12 h. The reaction liquid was filtered through Celite, diluted with ethyl acetate, and then washed with a saturated aqueous solution of sodium sulfite. The separated organic layer was dried over magnesium sulfate and the solvent was evaporated off. The residue was purified by silica gel column chromatography to obtain the intermediate C2 (10.96 g, 70% yield). The intermediate C2 (6.26 g, 20 mmol), 3-bromobenzamidine hydrochloride (4.71 g, 20 mmol), and sodium hydroxide (0.88 g, 22 mmol) were allowed to react in ethanol (200 mL) for 8 h by refluxing under heating. The powder formed was collected by filtration, washed with methanol, and vacuum-dried. The dried powder was allowed to react with 2,3-dichloro-5,6-dicyano-p-benzoquinone (DDQ) (9.08 g, 40 mmol) in o-dichlorobenzene (80 mL) for one hour at room temperature and then for 5 h at 120° C. After cooling to room temperature, the reaction solution was purified by silica gel column chromatography to obtain the benzoquinazoline intermediate B2 (6.37 g, 65% yield).

Under argon atmosphere, the bicarbazolyl intermediate A1 (2.57 g, 6.3 mmol), the benzoquinazoline intermediate B2 (2.94 g, 6.0 mmol), tris(dibenzylideneacetone) dipalladium (Pd₂(dba)₃) (55 mg, 0.06 mmol), 4,5′-bis(diphenylphosphino)-9,9′-dimethylxanthene (XantPhos) (69 mg, 0.12 mmol), sodium t-butoxide (0.86 g, 9.0 mmol), and anhydrous xylene(60 mL) were successively mixed, and the resultant mixture was refluxed for 12 h under heating. After cooling to room temperature, the reaction solution was filtered to remove insolubles and then the organic solvent was evaporated off under reduced pressure. The obtained residue was purified by silica gel column chromatography to obtain the compound H-2 (2.75 g, 80% yield).

The results of HPLC and FD-MS analysis of the compound H-2 are shown below.

HPLC: 99.67% Purity

FD-MS: calcd for C₈₄H₅₂N₆=1144.

found m/z=1144 (M+, 100).

### Example 1

### Cleaning of Substrate

A glass substrate of 25 mm×25 mm×1.1 mm thickness having an ITO transparent electrode (product of Geomatec Company) was cleaned by ultrasonic cleaning in isopropyl alcohol for 5 min and then UV ozone cleaning for 5 min.

### Formation of Underlayers

Clevious AI4083 (tradename) manufactured by Heraeus as a hole transporting material was spin-coated on the ITO substrate to form a hole transporting layer with a thickness of 30 nm. Thereafter, unnecessary portion was removed by acetone and then a base substrate was produced by baking in air for 10 min on a hot plate at 200° C.

### Formation of Light Emitting Layer

A 1.6% by mass toluene solution containing the compound H-1 obtained in Synthesis Example 1 as a host material and the following compound D-1 as a dopant material was prepared in a mixing ratio of compound H-1:compound D-1=90:10 by mass. The toluene solution was spin-coated on the base substrate into a thickness of 50 nm. Thereafter, unnecessary portion was removed by toluene and then the coated film was dried under heating at 150° C. on a hot plate to obtain a coat-laminated substrate having a light emitting layer. The film-forming operations for forming the light emitting layer were all conducted in a glove box under a nitrogen atmosphere.

### Vapor Deposition and Sealing

The coat-laminated substrate was conveyed into a vapor deposition chamber and the following compound ET-1 as an electron transporting material was vapor-deposited into a thickness of 50 nm to form an electron transporting layer. Then, lithium fluoride was vapor-deposited into a thickness of 1 nm and aluminum was vapor-deposited into a thickness of 80 nm. After completing all the vapor deposition processes, the substrate with laminated films was sealed with a bored glass in a glove box under a nitrogen atmosphere to produce an organic EL device.

By driving at a direct current, the obtained organic EL device was allowed to emit light to measure the external quantum efficiency (EQE) at a current density of 10 mA/cm². The result is shown in Table 1.

### Example 2

An organic EL device was produced and evaluated in the same manner as in Example 1 except for using the compound H-2 obtained in Synthesis Example 2 as a host material. The result is shown in Table 1.

### Comparative Example 1

An organic EL device was produced and evaluated in the same manner as in Example 1 except for using the comparative compound H-a as a host material. The result is shown in Table 1.

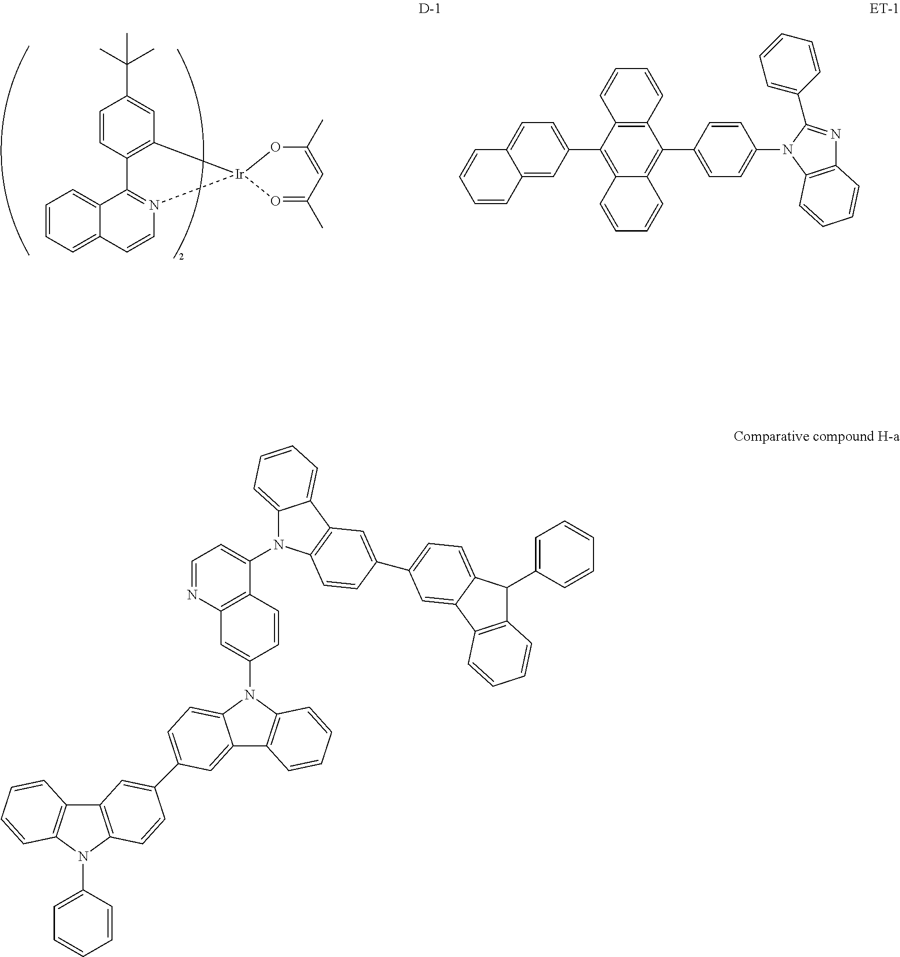

TABLE 1

| | | | External quantum |
|---|---|---|---|
| | Host material | Dopant material | efficiency (%) |
| Example 1 | H-1 | D-1 | 5.4 |
| Example 2 | H-2 | D-1 | 4.2 |
| Comparative | H-a | D-1 | 1.8 |
| Example 1 | | | |

The results of the examples show that the compounds having the molecular structure of the invention have the properties useful as a material for organic EL device.

### REFERENCE SIGNS LIST

1: Organic EL device
2: Substrate
3: Anode
4: Cathode
5: Light emitting layer
6: Anode-side organic thin film layer
7: Cathode-side organic thin film layer
10: Emission unit

1: A compound represented by formula (1): 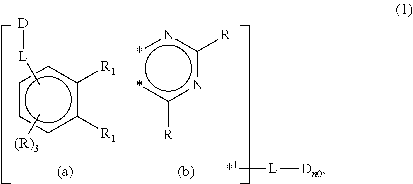
wherein:
two R₁'s are single bonds which are respectively bonded to two asterisks * of formula (b), or two R₁'s are bonded to each other to form a 5-membered ring, a 6-membered ring, or a fused ring, wherein two adjacent ring carbon atoms of the 5-membered ring, the 6-membered ring, or the fused ring are respectively bonded to two asterisks * of formula (b);
an unshared ring atom of the 5-membered ring is selected from a carbon atom, an oxygen atom, and a sulfur atom, wherein the carbon atom has two R's;
when the 6-membered ring is a non-aromatic ring, an unshared ring atom of the non-aromatic ring is selected from a carbon atom, an oxygen atom, and a sulfur atom, wherein the carbon atom has two R's;
when the 6-membered ring is an aromatic ring, an unshared ring atom of the aromatic ring is selected from a carbon atom and a nitrogen atom, and the carbon atom has one R;
when the fused ring comprises a non-aromatic ring, an unshared ring atom of the non-aromatic ring is selected from a carbon atom, an oxygen atom, and a sulfur atom, wherein the carbon atom has two R's;
when the fused ring comprises an aromatic ring, an unshared ring atom of the aromatic ring is selected from a carbon atom and a nitrogen atom, and the carbon atom has one R;
each of R's is independently a hydrogen atom, a substituent, or a single bond bonded to *¹-L-Dₙ₀, and neighboring or adjacent two R's may be bonded to each other to form a ring which may have one or more substituents R, provided that at least one of R's in formula (1) is a single bond bonded to *¹-L-Dₙ₀;
each of L's is independently a single bond or a substituted or unsubstituted arylene group having 6 to 60 ring carbon atoms, when L is a single bond n0 is 1, and when L is a substituted or unsubstituted arylene group having 6 to 60 ring carbon atoms n0 is an integer of 1 to 10;
D is a monovalent residue of a structure represented by formula (2) which comprises at least one carbazole structure: 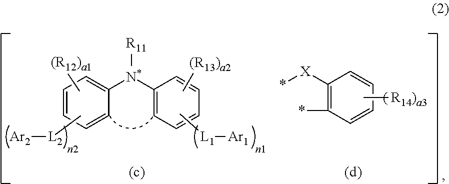
wherein:
a dotted line means that two carbon atoms at both ends thereof are bonded to each other via a single bond or the two carbon atoms are not bonded;
each of L₁ and L₂ is independently a single bond or a substituted or unsubstituted arylene group having 6 to 60 ring carbon atoms;
each of Ar₁ and Ar₂ is independently a substituted or unsubstituted monovalent residue of an aromatic hydrocarbon ring having 6 to 60 ring carbon atoms;
each of n1 and n2 is independently an integer of 0 to 4;
formula (d) bonds to adjacent two ring carbon atoms of at least one ring selected from two benzene rings bonded to N* and the aromatic hydrocarbon rings represented by Ar₁ and Ar₂, provided that when formula (d) is bonded to the aromatic hydrocarbon ring represented by Ar₁ or Ar₂, each of Ar₁ and Ar₂ represents a monovalent residue of a structure formed by the aromatic hydrocarbon ring and formula (d) which are bonded to each other;
X is O, S, PR₁₅, SiR₁₆R₁₇, CR₁₈R₁₉, or NR₂₀, and when two or more formulae (d) are bonded, two or more X's may be the same or different;
each of R₁₁ to R₂₀ is independently a hydrogen atom or a substituent; and
each of a1, a2, and a3 is independently an integer of 0 to 4.

2: The compound according to claim 1, wherein the compound is represented by formula (3) or (4): 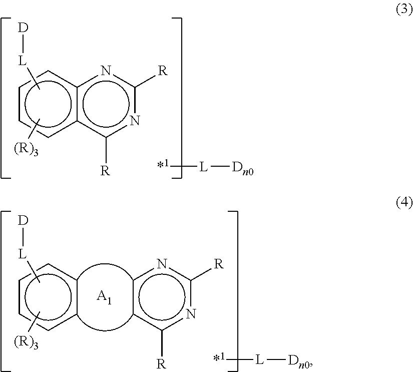
wherein:
R, L, D, and n0 are the same as defined in formula (1);
A₁ is a 5-membered ring, a 6-membered ring, or a fused ring;
an unshared ring atom of the 5-membered ring is selected from a carbon atom, an oxygen atom, and a sulfur atom, wherein the carbon atom has two R's;
when the 6-membered ring is a non-aromatic ring, an unshared ring atom of the non-aromatic ring is selected from a carbon atom, an oxygen atom, and a sulfur atom, wherein the carbon atom has two R′s;
when the 6-membered ring is an aromatic ring, an unshared ring atom of the aromatic ring is selected from a carbon atom and a nitrogen atom, and the carbon atom has one R;
when the fused ring comprises a non-aromatic ring, an unshared ring atom of the non-aromatic ring is selected from a carbon atom, an oxygen atom, and a sulfur atom, wherein the carbon atom has two R's; and
when the fused ring comprises an aromatic ring, an unshared ring atom of the aromatic ring is selected from a carbon atom and a nitrogen atom, and the carbon atom has one R.

3: The compound according to claim 1, wherein a structure which is formed by formula (a) and formula (b) in formula (1) is represented by formula (5): 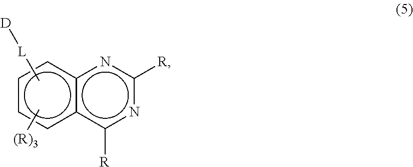
wherein R, L, and D are the same as defined in formula (1).

4: The compound according to claim 1, wherein a structure which is formed by formula (a) and formula (b) in formula (1) is represented by formula (5′): 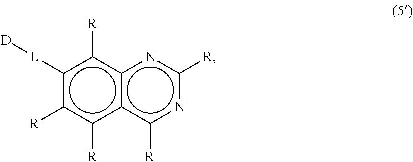
wherein R, L, and D are the same as defined in formula (1).

5: The compound according to claim 1, wherein a structure which is formed by formula (a) and formula (b) in formula (1) is represented by any of formulae (6) to (10): 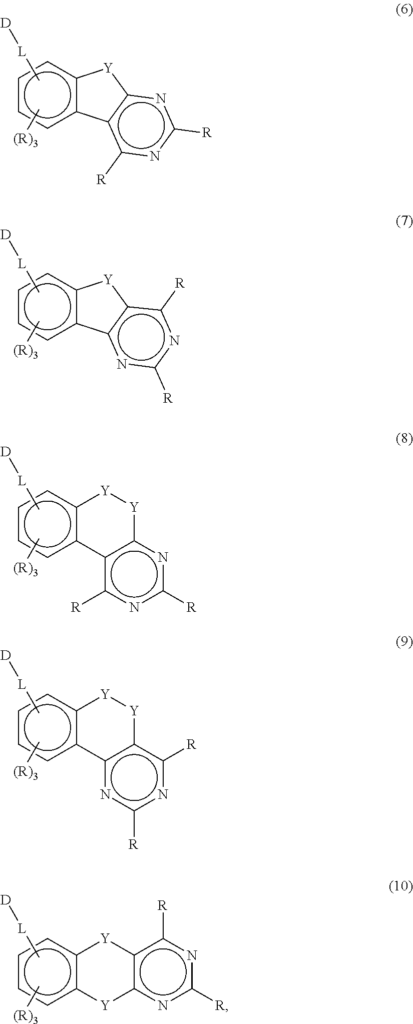
wherein
Y is selected from a carbon atom, an oxygen atom, and a sulfur atom, wherein the carbon atom has two R's; and
R, L, and D are the same as defined in formula (1).

6: The compound according to claim 1, wherein a structure which is formed by formula (a) and formula (b) in formula (1) is represented by formula (6) or (7): 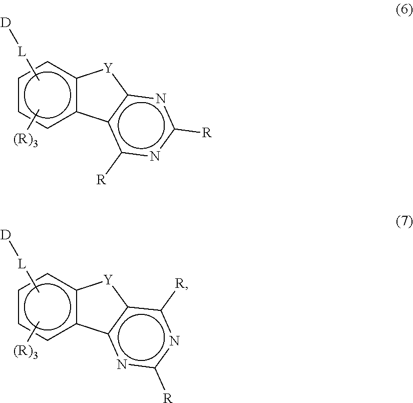
wherein:
Y is selected from a carbon atom, an oxygen atom, and a sulfur atom, wherein the carbon atom has two R's; and
R, L, and D are the same as defined in formula (1).

7: The compound according to claim 1, wherein a structure which is formed by formula (a) and formula (b) in formula (1) is represented by formula (6′) or (7′): 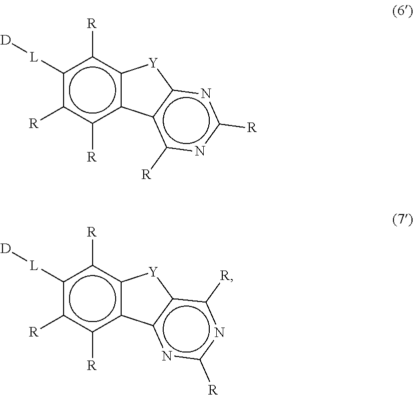
wherein:
Y is selected from a carbon atom, an oxygen atom, and a sulfur atom, wherein the carbon atom has two R's; and
R, L, and D are the same as defined in formula (1).

8: The compound according to claim 1, wherein a structure which is formed by formula (a) and formula (b) in formula (1) is represented by any of formulae (11) to (14): 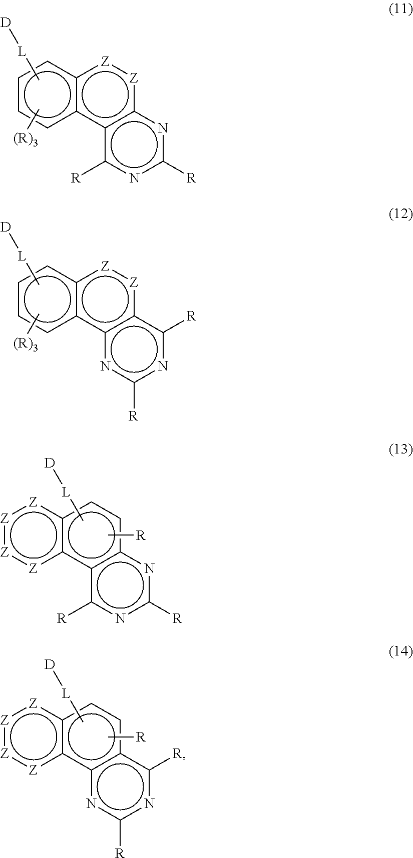
wherein:
Z is selected from a carbon atom and a nitrogen atom, wherein the carbon atom has one R; and
R, L, and D are the same as defined in formula (1).

9: The compound according to claim 1, wherein a structure which is formed by formula (a) and formula (b) in formula (1) is represented by formula (11) or (12): 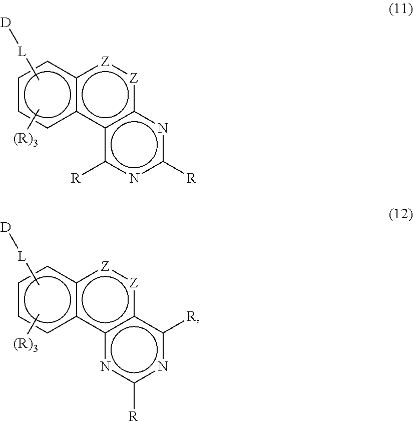
wherein:
Z is selected from a carbon atom and a nitrogen atom, wherein the carbon atom has one R; and
R, L, and D are the same as defined in formula (1).

10: The compound according to claim 1, wherein a structure which is formed by formula (a) and formula (b) in formula (1) is represented by formula (13) or (14): 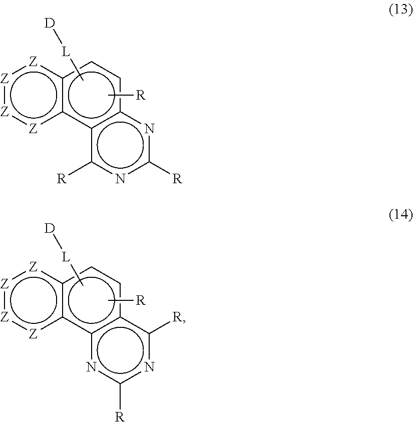
wherein:
Z is selected from a carbon atom and a nitrogen atom, wherein the carbon atom has one R; and
R, L, and D are the same as defined in formula (1).

11: The compound according to claim 1, wherein a structure which is formed by formula (a) and formula (b) in formula (1) is represented by formula (11′) or (12′): 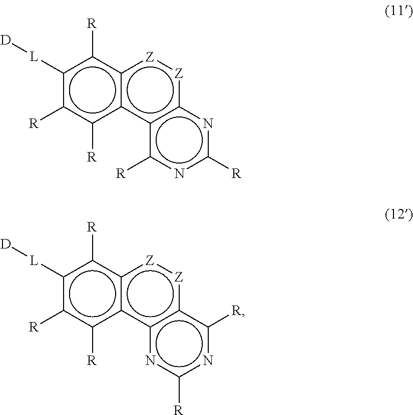
wherein:
Z is selected from a carbon atom and a nitrogen atom, wherein the carbon atom has one R; and
R, L, and D are the same as defined in formula (1).

12: The compound according to claim 1, wherein a structure which is formed by formula (a) and formula (b) in formula (1) is represented by formula (15) or (16): 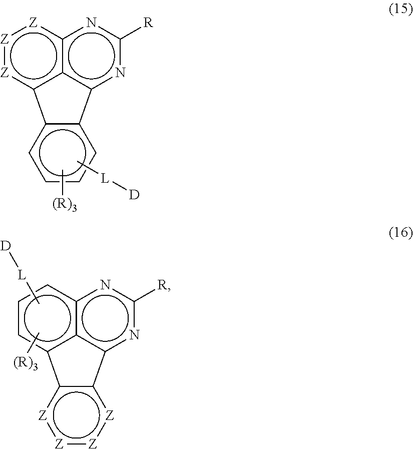
wherein:
Z is selected from a carbon atom and a nitrogen atom, wherein the carbon atom has one R; and
R, L, and D are the same as defined in formula (1).

13: The compound according to claim 1, wherein a structure which is formed by formula (a) and formula (b) in formula (1) is represented by any of the following formulae: 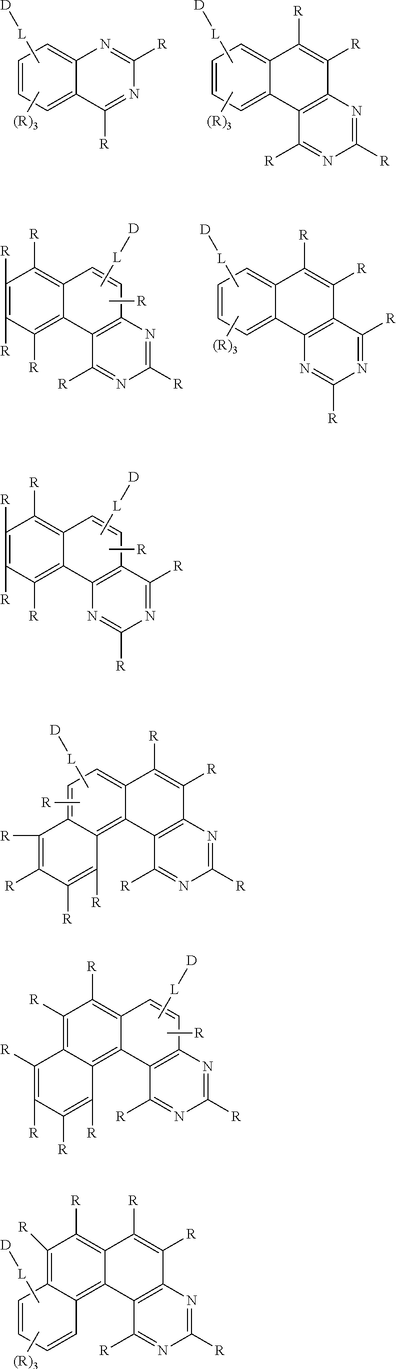 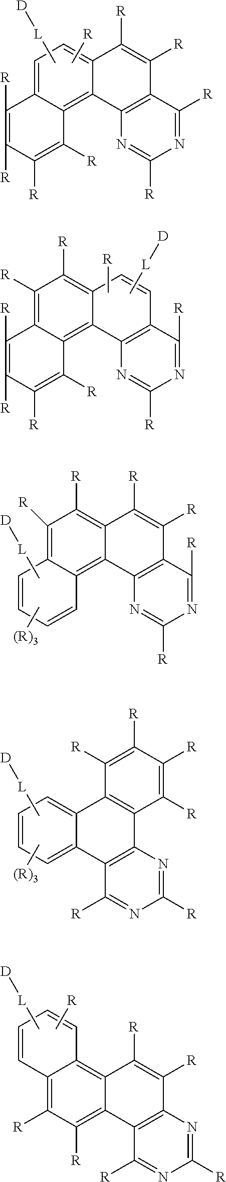 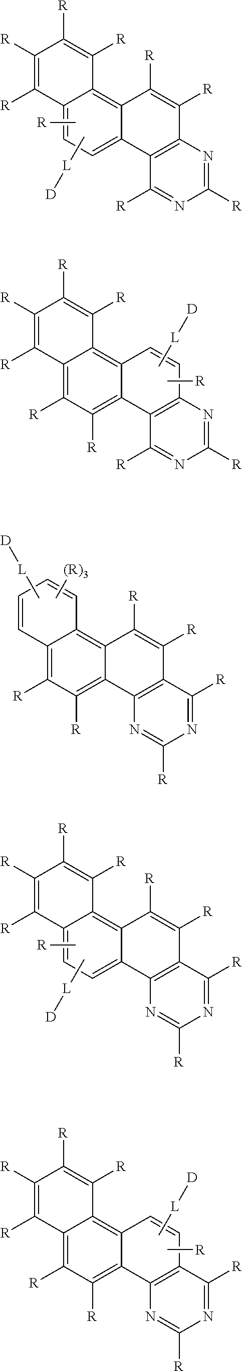 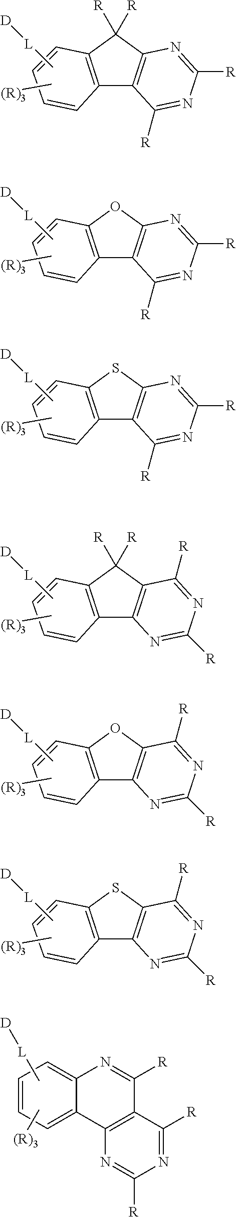 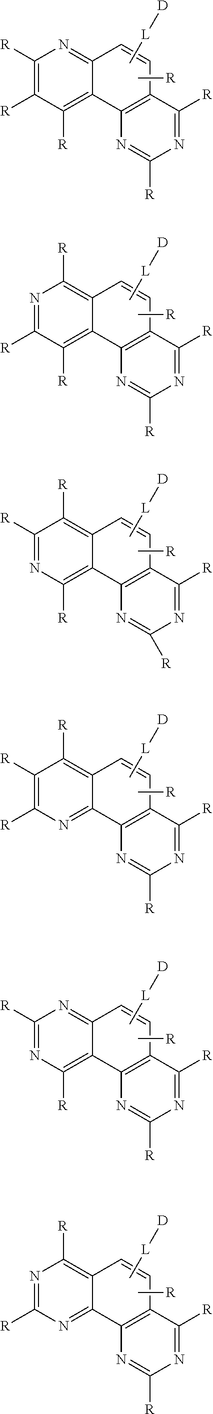 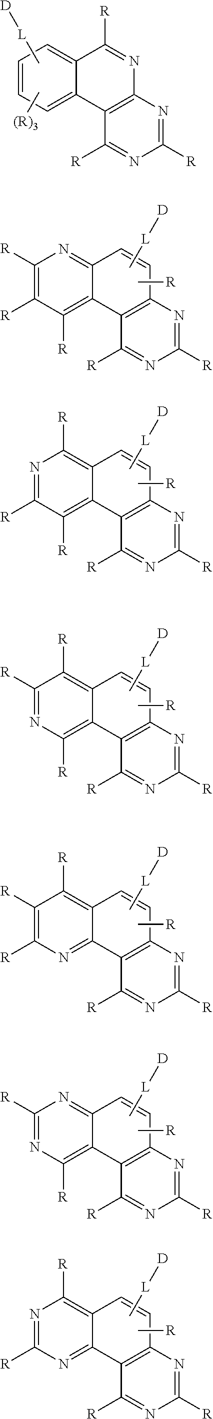 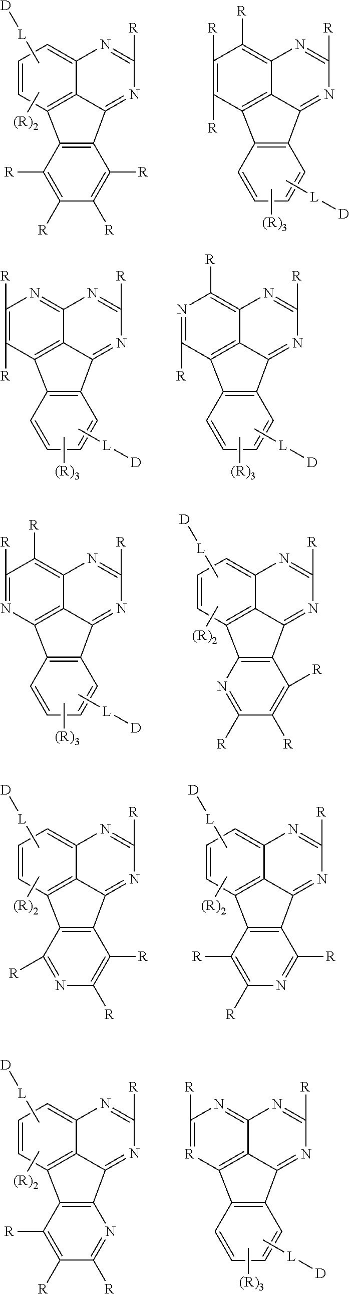 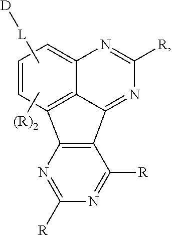
wherein R, L, and D are the same as defined in formula (1).

14: The compound according to claim 1, wherein a structure represented by formula (2) is represented by any of formulae (51) to (59): 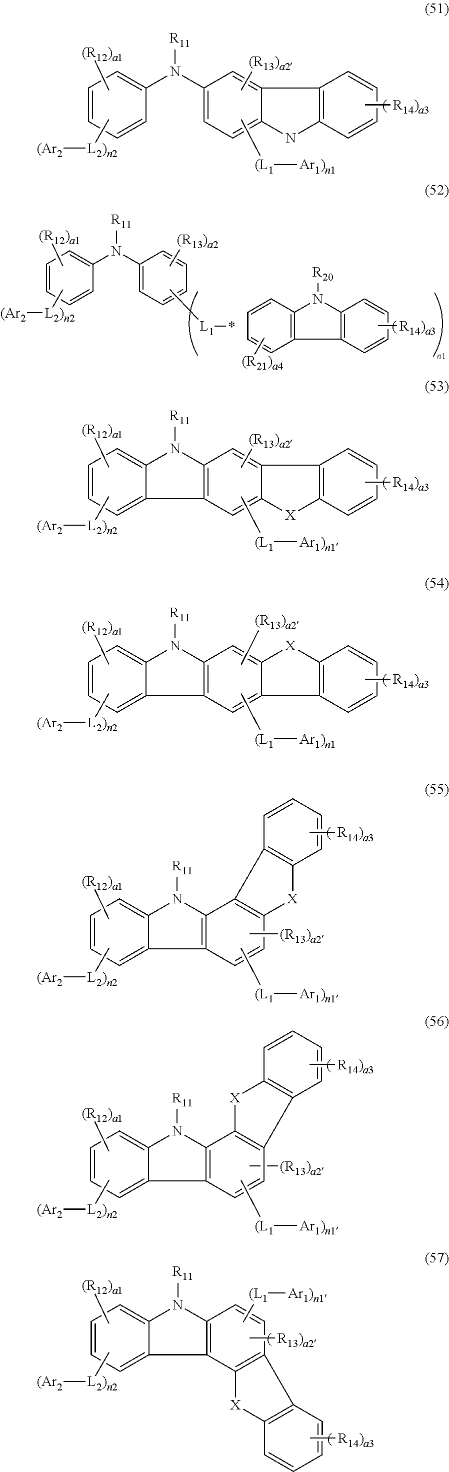 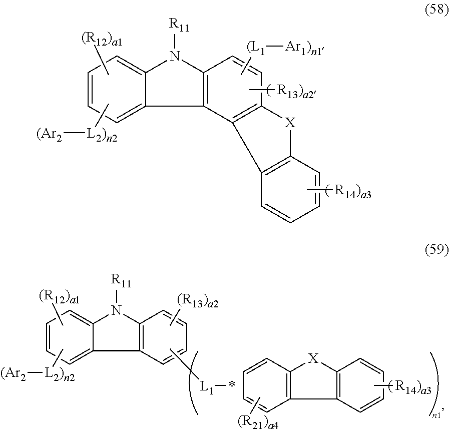
wherein:
X, R₁₁ to R₁₄, R₂₀, a1 to a3, n1 to n2, L₁, L₂, Ar₁, and Ar₂ are the same as defined above;
R₂₁ is a hydrogen atom or a substituent;
a4 is an integer of 0 to 4; and
each of a2′ and n1′ is an integer of 0 to 2.

15: The compound according to claim 1, wherein a structure represented by formula (2) is represented by formula (60) or (61): 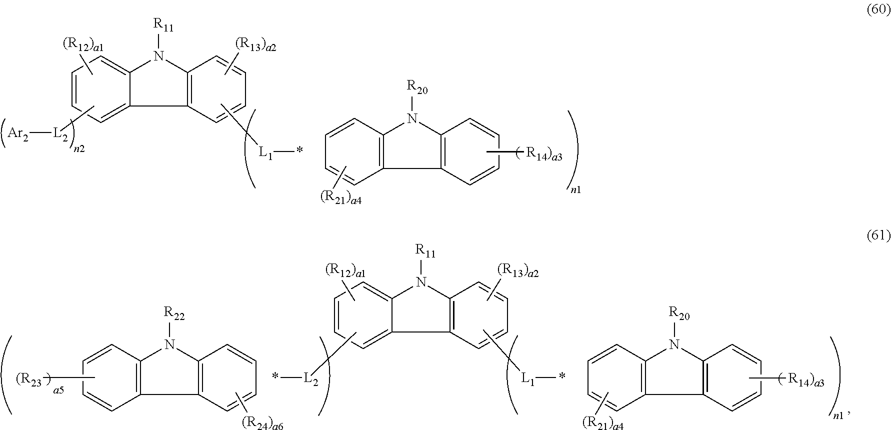
wherein:
R₁₁ to R₁₄, R₂₀, a1 to a3, n1, n2, L₁, L₂, and Ar₂ are the same as defined above;
R₂₁ to R₂₄ is a hydrogen atom or a substituent; and
each of a4 to a6 is an integer of 0 to 4.

16: A material for organic electroluminescence device, comprising the compound according to claim 1.

17: An ink composition, comprising a solvent and the compound according to claim 1 dissolved in the solvent.

18: An organic electroluminescence device, comprising an organic thin film layer comprising one or more layers between a cathode and an anode, wherein the organic thin film layer comprises a light emitting layer and at least one layer of the organic thin film layer comprises the compound according to claim 1.

19: The organic electroluminescence device according to claim 18, wherein the light emitting layer comprises the compound represented by formula (1) as a host material.

20: The organic electroluminescence device according to claim 18, wherein the light emitting layer comprises a phosphorescent emitting material.

21: The organic electroluminescence device according to claim 18, wherein the organic electroluminescence device comprises an electron transporting layer between the cathode and the light emitting layer, and the electron transporting layer comprises the compound represented by formula (1).

22: The organic electroluminescence device according to claim 18, wherein the organic electroluminescence device comprises a hole transporting layer between the anode and the light emitting layer, and the hole transporting layer comprises the compound represented by formula (1).

23: An electronic device, comprising the organic electroluminescence device according to claim 18.
